SEQUENCE LISTING (286 sequences)

SEQ ID NO: 1 - protein, 606 aa, synthetic construct
MSQHICELDELYSMMVFNLENMFEKFSLQFDDRIFAKRGETVDVIQINALLCNAVSAGRTLIESMEKFDEFYISKDKSFKKNFISKAYDQYSEYKIVDFLRNYMQHGHIPIHYDEEKIYLDLSEILETTHLKMNKNLKRMLQKAKKDLLEYGVADTRLCCVPLFYKYFLLIHRLYRAFYSYAEYTLMQIGEEKRKLLQDHPEYVRQVDEIAFAPVYQDELGQLHGVAVEDGYEEKIRENITYAEEKLQEYIKGNGQICSLQIDYCLEYRIPEMILIHEEELSENLVSYCKKHGHEIRHVSFYTYYKDDMDSYTRYKMFPYIQFEESVEWNVPYDRVTIRDFLRTFPEAEEKGILVQANNMGGDGIQIAQAVLQGWKTFLYHSSQILDTLGINSLADAIDWASRVVFIYQSIGWLKKSFGKRIEKKPTIEQLEEYIRRAERWELSQLSSTLHAAPELLKLVLSEVGYISQDGELFVYDEVIATQRKEEERKRKAEKENSHGTQVDCRKMNKVIEELNVTILYYASLQNEKKAEECGKETRIGKCVEQVICKYREFLWWDEVREELKVRDPLLEKFTEEIQGKICRDVRALEEELSGKCRELEKNESL

SEQ ID NO: 2 - protein, 1363 aa, synthetic construct
MPFTAETKRAYTLRLNGVNKKDNVWKEELWKTHETVNEGSRIFGDLWLTLRGGLSAKLAETKEDSSTRRILLALSWLTVEAPADLIPPEYIIATSKQPRDEREVALVKVLKSILKSQGVKNISDWENDCKASLASDIKEDAVWVNRAQIFADKKKSVGWNSNEAESFVFDMFGGQEAYFSLGGKEDKEADSKDFCQKAGNWLSTNWGSGVKSDKSLIIEKLSLITSLDLLSLKKKNSSAILVKIIKALEENYKPLDEEYKEGEDPKDTFSKISRIIGWKGSPSAGFRILEKMTKDAPVSVEMVEDLLIRIKKEIEDKQKKLGKPKIKWANKIKAEFESNVHFAFRGERDHQKEFSVLMDHALRRVSGCHSWVKIAEVSRRDFDADANKISDKLISDWLDEYTDQRSSDSGSLEGYFVRKNAIDGWDDVVKAWSELGKISTPEQRVYAVRALQGELEKFGDAQLFEELAKEKPELIWYRSGKANPDLFKQEVSKRIALHNKTRFKVPAYRHPDPLLHPVFCDFGNSRWAIAYGAQDKVSSLNERDVFLTLWNGKEMTKTTLRWQSKRFFQEIVSCSKNQKGTPVSRNNPQALAASGVQGNSSIIINDLFDRKDWSGRLQADRAQLEKAVTLRKKGGTKFKKFIKEIRWFLTFSVPLKPSGPWIEYASQSSYLHQNRSDGKANKPLVNLGPKNERDEIRGLAYPLDHPEQTNLRKGMARMHLSRLEGLRTLSVDLGHRHAVATAVWLTLSIKSAINKFPWIDKAEFSSSKLFLQMEKDGKRFVFRRIGANKLPDGSIHPAPWAYLERQFFINLAGENSESRFATDTDRLGVKKFETWLGYYTSEPRNSKRWKMDELFAELVKIARFGLARHAKRARISEGLVAKVKIISGDRTIPLDAVSRKEHITNLLEEYHLLVTSPNWKDDEGLKGWSQLTKGILAEEELKISTQDSAPKKKLDKTLKEKIIRVSEKLIQDSSLCKKLSNTLKIRWEKDDDKWPEFIKWLSRSLMPRSVKDLKSIRQKGGLSLLRVTTIEDFRRKVQLAYFTRKKPDGQRATVGLDFASKALKVIEKLRENRVRQIASFITSSALGISMTNNLVRYEPCQAVVIEDLTNYRPEVTQTRRENRQLMQWSSGKIKKLLAEACQLSGLHLRQVSPKYTSRQDSRTGSPGIRCVEVSVEKLISAKRWQIAEQKAMEKRAAGKAMASDFYFLSLLDRVRNKKLSGTVLIPQRGGEIFVSSNDNGPIALQADLNAAANIGLVALLDPDWSGRWWYVLASKKDHVVDVTGSSYFENSPKLSVLNDEKVESDHIKKSSDSGKSYHFRDLMLGKPNAGEWFDSFPKYEASFTLRAISRLEQKYGIQEETPF

SEQ ID NO: 3 - protein, 1343 aa, synthetic construct
MNRIYQGRVSKVETFTGDKENLWQPLDPDPKIAREKWQSLLWQHHELFQTDVNYLLAAFAAQVPDNCEDEIWSDYRSAIERSWESYTGRQGTWKRPFEYVCVLVGCKKNASFEEFRRKLNSMTGSKASEKQRLEALKQLFESATDTAMKLTDPDQPVEESLKGKAKDLFGSTLVNLCAQKTKVTPRDVIAEQRNHASECTKKANEDGHLKWADVFFFKTDKSSSAWSREDAGKNIVQCLEKLVAEVKQKAKDAKTVEQETKLTELAGRLEKQKTPLTTWCNNPKTDLPTTEPTRKGSGGYDLKAAILFSLQPDLDGFREAFLLFNQGRIKEEFTTKAKPDAAFIARMAGGVARPVFPFFCDLWAGKFNDEKVGQGIWPDFEKQAFSEVFTKIGQFIVRGRKFELRLAIANATIDRIKKQKKSDPRLQAVERITETLADALPDCAVDENGQRIPYAIRERTLKAWPKVRTAWRDTIKKSPDVSADDLIKQKNRMQERMREKFGSAPLFDLLVKERDIWNHADEDPLKIWSEYVEAGEEKTHLETARLFAPAHATLSPRFFRWSETNNKEHLTASAVEIPFAFEADALDLDKKEKIKIKIHFWSPRLLRDGLRSKNEKLDKDEPDQTWMPPVLRAFVKMRKWPSDKQTFAGASVRLAPRCKENIQLVFEPALDNNTLRKVWAEEMPFTPRMEKLNDEWVAAGLRWPKSEAEKFPWLESGGVKCLAADLGLTNSAAWQILQATNKEKAANTTRLRHRLNPDSEKPAWFTHSIANGIVRVSGEDRWGWRKFASTEKAEIRAELKQPTDKRNALCRKFSALNREIEFQTATHAFLPELSGSGGRNATDEETSEAEKYFATFKTKGFDIIERRPNWKTQLSFPEQNDELLWGLKRVRAQLFRLNRWSEQLGNKKNSKPFQSAIEIIGGLREGDPLLELAALKSDTKNLKKRVGELAGEYLDCFKLMLPRIADRVLPWRRGHWSWKPCENDWHRMELDTSKPRPEALLAGQRGVSLARLNQLKDLRQLAQSLNHLCRHKQIERREIVPEPFEDCRQAMDDAREDRAKQIAHEIFAIALGVTLAPPPADKQERKQTESLHGVYRCLERGPVNFIALENLTAYKTSAKQGRRENRQLTSWSHRRVHKILGELCELVGLPIVLVDPKFTSRFSAKDHSAGFRTEEVRKDDTRRSFWKRKAMEESNGWQAEFLSWLDKLPNGKTLLLPKKGGEFFVSLGKDSSLFQADLNAAYRIGLRALAHRNRAELLGQIWIEKEKKYYLVDTANVFSDSVLRDGCSFKTVTSSEKLWEYVNEKLAWQRCREINQARLKTFGVVVEEPANNLHPDEEDDIPM

SEQ ID NO: 4 - protein, 314 aa, synthetic construct
MNSKERRKILLHKYVKEVEQLNIKLPEGYDDMGKGSFICPLCLRIFNIAQLEGLVNNFITIEHVPPKNLGGKPITLTCNDCNSTCGHELDVYLRNEMEHHERAYFNRSKGHFSKYSHGGVEVNAITNEDKDGTINIKIEKKHNSPFSFDTFCESIQSSADDLRIDGHLILGNHKRNTKMAEVALLKSSYLYAFYKFGYRYILSTKLNAIKLQIQNPNEDILPPYYILFNESHIPNNKPDEIYIFTLNGEKVLVVVLTLKSSNSNTLRRFATVLPMPGQEDLELYNRLKNIHETIGYIEVEYIGIARERGQIEMK

SEQ ID NO: 5 - protein, 316 aa, synthetic construct
MMVKYNAIVVMENLNQGFKRMRGGIAERSVYQKFEKMLIDKLNYFVIKNETWTNPGGVLNGYQLTNKVSTIKDIGNQCGFLFYVPAAYTSKIDPSTGFVNLLNFNKYKNSEDRRKLICSFDKICFVQNENLFKFSIDYGKLCPDSKIAIKKWDVFSYGTRIIKENLTTGHIEENPEYDPTEELKSLLFSRGIEYQKGQNLLETIPTSDMTREFWNSLFKIFKAILQMRNSLTNSPIDRLLSPVKGKDGTFFDTDKVEGTKFEKLKDADANGAYNIALKGLLVLEKNDSVESNKDLKNVKKISLEDWLKFVQITLRD

SEQ ID NO: 6 - protein, 373 aa, synthetic construct
MVITRKIEVFVCEDDKDLRKEYYDKIYKCRDIAVKTANLGVSHLFMLDNTTPYLSDDDRDKLTFLGCTGKKATKQNAPYVAASEKFKGQADMSMLSSVLQNVGKMYNDDKKKGGMWSKSLRSYKANMPIPFKASCYRNLRFSDYNDKEGKPHNGCFFTLMGIPFQCKFGKDRSGNRIIMQAVVEGKYKMCTSSLQIDGKKIFLLLCVDIPKKTVKLDENKTLYAFLGVMNPIVCTTDIKQKGDIDTDWKLWEIGTEAEFNYRRRQIQEALKRCQVNNRYSRGGHGRFAKTKAIERWRAVERNYVDTKLHTYSKMLIDLAVKHKCGKITLMNQLHREDAAKDDKFVLRNWSYHSLRTKIDYKAKMYGIKVEVEK

SEQ ID NO: 7 - protein, 377 aa, synthetic construct
MSLITRKIQIYVSEEDKELKKQFVHTIYDWRNQIRRAANIVVSHKFTQENIKDFVYLKDEIKEKFYVKDILKEGKGMSEQNTTYRVLADLLKGKIPSDIYSCLNQAVAKTFKETKSEIFKGETALRSYKNNIPVPFSSDSIRNLHWEEKDKRFYFTLFGIPFATALGADRSNNKVIIERVLSGEYKLCQSSIQIDDRKKKMFLLLCVDIPKKEVKLNPDKKLYAFLGVFNPIVCTTEITDDLDAKNPYKVWEIGTEEEFNHRRRQIQEAVKRCQINSRYTEGGKGIKKKTKAIDRFHEKEKNYIDTKLHTYSRMLVDLAIKNECSEIVLMKQKEREEMAKEDNQNGKPFVLRNWSYYGLKDKISYKAKMVGIGITVE

SEQ ID NO: 8 - protein, 377 aa, synthetic construct
MIITRKIEVFVCEDDKDLRRAYYEKLYANRDIAVKAANMAMSHLFALDNTMPYLSDTDKEKIEFLGVKGQKATKQNAPYVAASEAFKGQADMGMISCVLQNAQKMYQEDKKKGMWNKSLRSYKSNMPVPYKRERFLNLRFDDYINGNGEKRNGCFFTLIGVPFQMRFGRDRSGNRLIVERVVSGEYKMCTSSLQFDGKKIFLLLCVDIPQKNIELDAKKTLYAFLGVMNPIICTCDVKAAKEYDSGMKSFEIGTKEEFNYRRRQIQEAVRRCQINNTYSTGGKGRKKKTKAIERYHDKERNYVDTKLHTYSRMLVDIAIKHKCANIVLMNQTHRENYAKEDNKNGEPFVLRNWSYYGLKEKIEYKCKIAGIKLKEEK

SEQ ID NO: 9 - protein, 383 aa, synthetic construct
MVITRKIELVIPMMDMERRKAYYEKLYASRDIAVKVANMAVSHLFALDNTMPYLTDEAKQTIVFIGAKSGTVTKQNAPYVAASRAFKGQADMGMVSSVLQNVQKMYQDDRKKGGMWKKSLRSYKSNMPVPYKADRFLDLRFVTYVNGEGKEQEGCFFTLAGIPFQMRFGRDRSGNRIIVRRVISGEYKMCTSSIQIDGKKMFLLLCVDIPRQENRLDEKKTLYAYLDVMVPIRCTVSVKAANEEDSDDKCIMIGSKEEFNYRRRQIQEAVRRCQMNNSYTAGGKGRKRKCKAIERWHDKEKDYVDTKLHIYSRRLVNLAIKHKCGVIHLVNQKAREKAAKDRNAEGEPFVLRNWSYYGMKEKIAYKCKIAGIRLKADKEDYED

SEQ ID NO: 10 - protein, 408 aa, synthetic construct
MTQFQNSRLPHGRDLSDQTWLESLALATTQMPEDETQAADWQASLLTEPKPLPFPVGYETNEDLHWSLNAKGRLCVSFNGISEHSFEIYCDHRQLHWFQRFLEDQEIKRTSKGQHSSGLFALRSGRLAWRMGLGEGAPWNRHDLALFCAVDTRLWTAEGTAQVCQEKVTTINKIVKGTRAKGHLNQAQEAFLQRRETTLALLNHPFPRPSQPLDRGDANRLLGVSFGLDKPATVAIINGATGQVLTYRSMRQLLGENYQLLNRYRLRQPRIAHKRHNSQRKAAPNRQSEGGLGEYIDQLLAKAIIVIAKEFQVGSLVLPDVGDVRESIQSEIQARAEQKYLGSIELQRQYALQYRASVHRWSYARLSQKIQSQAVQCGMAIETAKQPLTGTPQAKAKGLAIAAYQARK

SEQ ID NO: 11 - protein, 429 aa, synthetic construct
MYTIELRLDTRGYDEYLEKKFKYGYKLKRALVNYFNMQENRRVNSDRYKELAERMKELNTRQDIIKDTKDKGRKKLLKEAYKELSEEVKKGWIELNNSYGLGDGKFVDYKKLGEASVMYERYASKGIINWSSFESMAQATKQGYLKRRKQANSDNTLKVPRYVDFNTLWYRKCNNNITPDGLRFGKRGNYIIFPYIFKGTDEIRFTYALERQKIAWYGIKRILDRHNKWLYSVLIVFSEVPYGLPESPVKQGKVVVSVDVDKLAIKTFNVDTEEEVLFDIANDFGYSEKLSYYDRKLEESRRQSNPDNYNVDGTVKEGKRSWVKTKNYNTLLARKRTLWHKIKQSRKDRFHSITKAIVLNCDEIVVIKEDFKALQKRKDYNPEEMKWIDSRKQKGAEIMFNAPYEFIELLKQKASYLGLSYSEITKEKQ

SEQ ID NO: 12 - protein, 471 aa, synthetic construct
MARLFAAYDTTDDLLTRCAIAHLIKNGCKISEAEEDPEKFAHRIHRKQKEREQLEAQLQARLPKGRDLTGEDFLETLAIATQQLPASVSQSREWQAKLLTRPASLPYPILSDATELRWSKTAKGRIAVNFNDLDKFLKATDPDLQAWFKTQKEYPFRLYCDQRQLPLFQRFLTDWQAYQANKDDYPAGLLTLSSALLAWREGEGNGDPWNVNHLALHCSFETRLMTAEGTLAVQQEKVAKALKNLTHAKPDPRNHSTLKRLQHLPERPSKMPYQGHAEILVGLSVGLAHPITAAVVNGRTGEVLTVRTPRTLLGDRYPLLHRQRHRQQQNALKRHKNQKRGVAYQPSEAELGQYVDRLLAKAIIQLAQAYQTGSIVIPNLTHLRELLNSEITAKAEQRCPGSVEAQDRYARDYRVSIHRWSYNRLMAAICSNAQQLGITIESGFQPLQGDPQTQAKDVAIAAYHARAIATR

SEQ ID NO: 13 - protein, 492 aa, synthetic construct
MPGWQELANGDETRLLQLREALKQHIIEQRQRIQRELLRLTGRILPLRGRDWVWAPHPDKPDCHLLRQAREDARRQKVKLRGQRGLSMARIEQLSELRRRWQSLNQALRRKIGDRPLTASEMRNDPIPDPCPDILIKLENIREQRVNQTAHLILAQALGLRLRQPQLSPESRQAADIHGEYDVVRPPVDMIVVEDLSRYLSNQGRAKAENTRLMKWCHRAIILKLNMLAKPFGIPVLETPAAYSSRFCCLSGAAGFRAAEVSWKDRHEFRWRVLLGEEAKARAEGAEPSPSAMHAARLFSSLEKIAASERPNRTLLAPQPGGPIFVTAKAIPHPAPAGNRKLSGNKDILPMQADLNAAVNLALRAVAHPSVADIHHRLRTERKKGEKGQPDTFIVREPRRFGKDKVPIQIRDGDSLPKERNPNLFYDRHEIAPFGRARLETDSAGRFDYASGPGVWKTVNDREYQWRRCEAINNERLRRWLNHEQVKDDVPM

SEQ ID NO: 14 - protein, 515 aa, synthetic construct
MVTNRRIEFKVSLDQQQQQIFDHHIAKLQAVWNIGLACIEWREWWEKWQQVLESPDHPDYAPDAVPMGWGHNSIKGKKKPVYGLCCDRVKFRLEKNDPILDVGEWVEGTKKGSGDSIAGYWVAYPAKPDPVKEHWVEDPRLPKHGQDPYMSLVSMFGKKYWPSDHFIQGLSSAWIKGQCKNLATAWKAYKKGIRKRPQFKKRHEAGNSLLCVTRIPLKQNGMTLPGMGFVRIPKLAQRWDSIKDPCPISLLREGSRYYVQLTATLEQRPVRSNGVTVGIDPGSVHVATDDRGRMIDSPRYAKKQERRKRRLQRSMARKFRLNTTTVYDEDGRVLRHEPRKGWKRRNHAKVKQQLQSLEAHTKRARRAFNHFHSTRIVQSADVIVLENLQLQNIGAKVKTGKTGISNGRKRKTGLNNKLRDNAIGQLFAMVEQKAAAAGKLTTRVNPFRTSMTCNRCGFSDKANRVSQAVFHCQSCGHRANADQNAAANIRKMGLLGVTKLTFDNDWRITVPNWHD

SEQ ID NO: 15 - protein, 558 aa, synthetic construct
MTSNEATTKKIAEFKLSPTIEQSAQIDMWLTQLKWVWNVGLSLLEEDQQRYWRAKQEWAEYSPEVWQWRKSCKIDGIEYVNPDNQYGLCCAYYPDKCDIRKHRDIEEPFKHVKNYRFIANMRCPDFLHPIDSESRRSVVDNLIDAWTQYRKGKKGRPRYKKKADKLESFSNGNGRIYVNISPLGNDNALIDYPKLGSIKCKGFYRRYAGNHTIVRIVKKADGYYVQFTSTVDVKPVKPAKKQEVGLDFGVVAPIADSDGRLHKSKRYAKLQAKKLARIQRSISRSKDGSNRLKAKRSKLAKVHLKIARQRDRFIHKLTSKYVSEYQSIAIEDTKVKNMVRAPKAKAGENGAFLPNGAAAKAGLNKALLDVAIGKTKTMLETKSKASERIFVKVPAPHTSQGCSQCGYYSEEFRPSQAVFKCLNCGHGDNADSNASTVIRNVAFKGLVLKPYPASSGEFKGVDHATHGKKRQPSKTRKSKKGSVGETSTAINRESGAVNSLTTRANPENQSPQDFELWQDLKSLSKANSKLQSRKRSAQIDPNIGAQLGFWDLSGEASG

SEQ ID NO: 16 - protein, 571 aa, synthetic construct
MSSEKKPTKSEIRAAHREAKKKDFVTRVLRVKVIKPLDGDWDTLGKALRALEKPLHLVLNNTVNRLELAYARDRERALFQGLEPEKGTQHGRAYRFAVEEWERQVELATQRLAEETEASTQGDDKRKKKCYDWDADVVAFPPMRSSVIHGCSGHAFTQWSLYNKERLRGGRRLGGFSANCPISLTTGTSHEYVSIEARDGNAVLWVRLCKDYHPGLIVSLGRKRASQKSILKAILEKPDRLRSVKLVHVHDRHSRKWQWEAHIAYHSPRPQIEDKPRLATIARGMHSFITVSVARSSQSARDAWTRVLETGDDIIAHKRAYFSRRKSLGRHGRSLGRGCKGHGESRHYERLTRIGEDERKWVHSRCQQAAANAIKFLRNKKVTHILLEDWGSPNSENWHVRNWPWAELRTCIMEAAEKEGMVVDLIPSIGHSKTCPKCGSEGSTSKRKGSMFRCSNVTCGLYRPKDIVRAWNMLRHVGYDVPIEEANEETRKVVEAMTARLRKEGAVSPRKKKRTKKGGSTTPESADAQASAKSEQAGRNGKKRARIASRPPGLPTSAAKPPVKARTRGAR

SEQ ID NO: 17 - protein, 574 aa, synthetic construct
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 18 - protein, 587 aa, synthetic construct
MGKIPLKTVSETCKQLKQDAQFAGQPGRFYSSAITTVHRIFKSWLAIQTRLRNQIAGQTRWVAILQSDDELTIASQGDINTLRAKASNLLTELNGSTSQINELHSKRSGSKKKGKKEPEQSDASISRTLFKLYWKTEEPLSRCAIAYLLKNGCKLPNRDEDPKKFAKRRRKTEIRLERLTSTLQRTRIPKGRDLSWRTWLETLHEAETCFPKDENQAADWQAKLLTEPATLPFPINYETNEDLHWFHNEKGRLCVTFNGLGEHFFEIYCDQRDLHWFKRFLEDQETKKASGKQHSAGLFTLRSARISWQEGEGEGNPWVVHRLVLSCTVDTDTWTQEGTEKIRQKKAIECAKVIAGTKAKENLNKNQEAFIKRREKMLTLLDNPFPRPSRPLYQGQPSILAGVSYGLDKPATLAIVDIQTGKTITYRSIRQLLGENYKLLNQYRLRQQRNAHERHNNQQKGASNQLRESNLGKHLDRLIAHEIVAIAQKYQVSSLVLPDLSNIREVIQSEVQARAEQKILGLVELQRQYALQYRVNVHRWRYAQLSEFIQSQAAQIGISVEIVKQPLADTPQEKAKILAIAAYQSRK

SEQ ID NO: 19 - protein, 601 aa, synthetic construct
MSKLTIQCRLVACEDTRRQVWEMMAGRYAPLIATTLEQVSQHKDFPQWVSAGEIPAQVVKNLVNQAQSGLPARWCASAQRQVQETYKAWLTKRRKLQQKLQGQQTWLSVLRPDAELAQEAGLSLEEMKIRAQALLHREINNWFQVYQQCQDVVERSIFAYLLKHRLTVPTEPEDTDKLRRKRRQVEIKIERLETQLAGRSPQGRDLTGSRYAAALNEGEQCYWENDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 20 - protein, 614 aa, synthetic construct
MAEKNTPLTNELLHQVGHHCEFESWRKKGKIPSGIVKELCQPLKTDPRFTGQPGRFYTSAIALVSYIYKSWLVLMKRSHYKLEGKTRWLEMLSSDAELVERCGISVDGIRARASEILAQLALPDSADTGQVKGRKAKKSKKFQQQQDRDRNLSRTLFEAYRDTEDILTRCAICYLLKNGCQVSDREENSEKFTQRRRQVEIQIERLKEKLAARIPKGRDLTDAKWLETLFLATSHAPESEAEAKLWQDCLLRQPSSVPFPVAYETSEDMTWFKNHKGRISVKFNGLSEHTFEVYCDSRQLHWFERFLEDQQIKRNNQNQYSSSLFTLRSSRIAWQEGKGKGEPWNFNHLTLYCSVDTRLWTAEGTQLVIEEKVEEIVKNITNTKDKGNLNEKQLAHIQRKNSTLARITNPFPRPSKSIYRGQSHILVGVSLGLEKPATVAVVDATTSNVIAYRSIKQLLGDNYRLLNRQRQQKQVLSHQRHLAQRLDALNSLGESELGQYVDRLLAKEIVAIAQTYCAGSIVLPSLGDMREQVQSEIQAKAEQKSDLIEVQQKYAKQYRASVHQWSYGRLISSIQSQAKKAGIAIEEAKQPIRGSPQQKAKELVIAAYNARKTS

SEQ ID NO: 21 - protein, 615 aa, synthetic construct
MEDDIVLALKIKLLPTKDQVLTLDELFGKWASICTRLGSRGLEQDDLSPPSEFNGKLHFSKTQLNSATGDIKTAREALDARCQKLNSQVVRLDRDVTALKEAIVDSNKREIDKTKETRFYPKAWVRTGQLIRKYYPLGYFKRRLEHDERKLKRMKETVEKLKHGKVHFSPRRITIWASSLRFRFGNDPQAVISLTDVGEPLSIKLQLKPDQELVGSSKKSADFYENAILNFLAYSIKARLFGFSNSERFLLKARKPEKVQKKEAMLRKKTENLPSKIIIVEKWIGRQLSIEEKSVIEEEFTSVLKGGTIENKKFVCLLSNLATSILIRSEPILLNKYGIIIRKPIGKAKTKNVYNIKSDEWEYYLQLGYSPVLSPKLQYEPKSFIGIDRGVTHSVALTLFDPVSKKFVVNLLYPNPVAKWKRRNKCLVRALQKLKRRIKSQNGETLHFNQMRKRLHSVENRAENYWHNLASQIIKLAYEHKAAIVLERLENLRQSGRKKTKYGQGLRYALSLFDYKKIASLISYKASRDGVPVYDVDPKYTSKTCSACLLHDRVGSEFPGDKYVRGIDGNFKRGYCPVCREINNNSKSEIDADLNAARVIALCVQKKRGMPRKVN

SEQ ID NO: 22 - protein, 616 aa, synthetic construct
MNTSIFDQLTNQYSLSKTLRFELIPIGKTKEWIEKNKIISKDKQIKEKYEKTKPYFNRLHCEFIQESLNKVELTKLDAYFRTYVQWKNNKEKNKENNDKIKMELTEIQKKLREEIVAAFDKTAQAWAKNLQNHKLKKTNHELLFEAEIFAILKQKYSDEEKTWIIDENNKKISIFDSWEKFTGYFKKFFETRKNFYKADGTETAIATRVIDHNLKIFCDNIINFEKIKNKVDWSKVEEHCNIELESVFKIDRYTEFILQDGINFYNKIIGGEVRENGTKYQGINECINNYRQQTGEKVSYFKKLDKQIASEKETSFFEKNISSDEELYQILHKLITESEKRINAFTQILQLFINNQERFNIENIYLTKESYNTIAYTWINEPNILNQAMQKSYKEKNIKIQTQNDEYIFPDFIPLNILKQALITAEQNAFKWKSKYENLESFPQTNAWNQFLYILYFEWNSILEGQTINPTTKEQTIPGYNTAKQAIRNFLTNVDSQEEKKIKIKQFADSALRAYQQAKWFAVEKNREWITEVELDDFYLDPEYGYKEHVWDNAYEQIVQTYNQIRNYLTKRPENKEKWKLTFENPTLAAGWDKNKEVDNSAVLFIKNNKYYLGIF

SEQ ID NO: 23 - protein, 626 aa, synthetic construct
MSQVTIQCRLVAPEEARQALWAIAAKKNTPLINELIQQVVRHSDFENWRQKGRHPARVVSGICSQLKGDARYSGQPARFFMSAEKVVNYIFKSWFQIQLRLQQQLSGKQKWLEILKSDEELTEICGQDLEKIREKAAQILKQIQQTTEFGEPAQNQASASRNLIRQRLFKQQNSAQQSLNRCAIAYLLKNGCQIPTNPEDPQKFAQRRRKTEIQIKKLQDQIEARIPQGRDLTGQVWLQTLLTATQTVPSNNQEQKRWQDRLLVGSNPIPFPILFETNEDLVWSLNDKGRICVHFNGFSEHVFQIYCDQRQLHWFKRFLEDQETKRQNKNHHTSALFALRSARLGWKEGNGNGYPWDKNHLTLYCTVDTRLWSAEGTEEVRQEKAANVAQTLTALNEKKPLSKTQAAYAKRLSSTLSKLNNPFDRPSQPCKTANLHIIVGLSLGWDKPLTLAVWDSNTQEILAYRSLRQLLGKDYDLFVRHRQQQQSQAHERHKAQRQGKDSRFGTSNLGEHIDRLLAKAVVAIAQHYSAGSIAVPKLDNIRSLLQAEIDTRAEQKIPGYIEGQKRYAKQYRRNIHRWSYARLIAQISSKAAQAGIAIEESKQPNIGSPQEKAKQVAIAAYALRKY

SEQ ID NO: 24 - protein, 627 aa, synthetic construct
MPRDGSSAHAEMRPDGRWREWAVHRFLRSRGDAPEIEGILRAYGEVPPLTRRCAATRDERGLELLGSSAHAEMRPTCVSTAHRWSWFLRSRGDAPPCTIGESDAVMVPPLTRRCALLQAQRRAAAAGSSAHAEMRRSRRTRSALAGGFLRSRGDAPGQGPRHVITDGVPPLTRRCAREPSLRLRLERGSSAHAEMRPAAPPLPATGRWFLRSRGDAPPSIKMPLKTRGVPPLTRRCASFSDFRHIIGAGSSAHAEMRRRGSGSRWVRWRFLRSRGDAPRSDSPSSSSARVPPLTRRCANTSSAMAYLFLGSSAHAEMRRSSPRITRRRNWFLRSRGDAPTRSPPTVTAFGVPPLTRRCAHHHRSLAPHARGSSAHAEMRRHRRSRGRRRGGFLRSRGDAPDVRDYVASWSPVPPLTRRCAAERQHRESLRLGSSAHAEMRLKRPACAFAMIRFLRSRGDAPPTSSPGSSGSSVPPLTRRCALATAGVLAGLSGSSAHAEMRRGRSRTSSGTSWFLRSRGDAPVQVVTYAVLALVPPLTRRCAPLADPPRRRWYGSSAHAEMRPADGSPGDSVSGFLRSRGDAPRAKSVNRLKCRVPPLTRRCAAKRRGLAKWASGSSAHAEMRRAPR

SEQ ID NO: 25 - protein, 634 aa, synthetic construct
MSIITIQCRLVAGEESLRYLWELMEKKNTPLINELLEQLGKHIDFETWLQKGKLPAEVVKTLCNSLKTNPCFAGQPGRLYSSAIALVDYIYKSWLALQQKRQRKIEKKERWLLILKSDGELEQESGCSLEVIRSKASEILTKVTAQSEPSQKQTKVKKGKRTKKGKTDKAPSTLFNKLLGIYEKTTDSLSRCVLAYLLKNGCQVSELEEDLEKLAHRRRKTEIEIERLNKQLKSRQPKGRDLTGHKWLEALEIATQNVPKDEDEAADWQAALLRKSSSVPFPINYGSSTDLTWFKNEKGRICVHFNGLGKYPFEIYCDRRQLHWFQRFLEDWQIHHDNKQQYSTGLFALCSARLVWLEGKEKGCPWNVHRLTLHCSIDTKAWTAQGTELIRSEKIAAADKEITNKEQKGELNEKQQERLQRKCSERQRLNNTFPRPSKPLYQGQPSILVGVSFGLEKPATVSVVDVTKGNVLAYRTVKQLLGDNYKLLTRRRQQQQSLSDQRHKAQKRGASNEFGESELGQYVDRLLAKAIVAIAKTYQAGSIVLPKLADMREIVESEIQARAEQKVSGYKEGQQKYAKQYRVSVHRWSYGRLSQCIHESAAKVGIAIEIGRQPIRGSPEEKARDLAIAAYHAR

SEQ ID NO: 26 - protein, 634 aa, synthetic construct
MSNITIQCKLVTTEVTRHYIWHLMAEKHTPLINELLKCIAQDSHFEEWCHAGKIPLEAVRKTCKQLQQNSQFIGQPGRFYSSATATVYRICKSWLALRTRLRNQIAGQTRWLAILQSNDELTIASQSDIDMLRAKARQLLTQLNHSDSQDNEPQPKKARSKKKESKQPGSAISSTLFTLYGETEEVLTRCAIAYLLKNGCKLPDRAEDPKKFAKRRRKTEIRLERLVKTLQRTRFPKGRDLSEHTWLETLTKAEGCVSKDDNEAADWQASLLTEPATLPFPINYETNEDLRWFLNEKGRLCVSFNGLGEHSFDIYCDQRDLHWFKRFLEDQQTKKASGDLHSASLFTLRSGRIAWQEGKGEGEPWNTHRLILSCTVDTDSWTQEGTEQIRQKKASECAKVIASTKVKENLSKNQEAFIKRRTTMLTLLDKPFFRPSRPLYQGQPSILAGVSYGLDKPATLAIVDIQTGKAITYRSIRQLLGENYKLLNRYRLRQQHNAHQRQKNQQKGAFNRFGESNSGEHLDRLIAHEIVAIAQKYQVSSLILPDLSNIREIVQSEVQAKAEQEIPGSVELQRQYALQYRASVHRWRHAQLSQYIGSLAAQAGISIEVVKQPFTGTPQEKAKKLAIAGYQSRK

SEQ ID NO: 27 - protein, 634 aa, synthetic construct; identical to SEQ ID NO: 25

SEQ ID NO: 28 - protein, 637 aa, synthetic construct
MSTITIQCKLVAIESTRHNLWHLMAEKHTPLINKLLKRIAQDSQFEEWCHMGKIPLKTVSETCKQLKQDAQFAGQPGRFYSSAITTVHRIFKSWLAIQTRLRNQIAGQTRWVAILQSDDELTIASQGDINTLRAKASNLLTELNGSTSQINELHSKRSGSKKKGKKEPEQSDASISRTLFKLYWKTEEPLSRCAIAYLLKNGCKLPNRDEDPKKFAKRRRKTEIRLERLTSTLQRTRIPKGRDLSWRTWLETLHEAETCFPKDENQAADWQAKLLTEPATLPFPINYETNEDLHWFHNEKGRLCVTFSGLGEHFFEIYCDQRDLHWFKRFLEDQETKKASGKQHSAGLFTLRSARISWQEGEGEGNPWVVHRLVLSCTVDTDTWTQEGTEKIRQKKAIECAKVIAGTKAKENLNKNQEAFIKRREKMLTLLDNPFPRPSRPLYQGQPSILAGVSYGLDKPATLAIVDIQTGKTITYRSIRQLLGENYKLLNQYRLRQQRNAHERHNNQQKGASNQLRESNLGKHLDRLIAHEIVAIAQKYQVSSLVLPDLSNIREVIQSEVQARAEQKILGLVELQRQYALQYRVNVHRWRYAQLSEFIQSQAAQIGISVEIVKQPLADTPQEKAKNLAIAAYQSRK

SEQ ID NO: 29 - protein, 638 aa, synthetic construct
MLPKVFFSDKNTSYFSPDEEIQRIRNHGTHTKNGDPQEGYEKLDFNLNDCHKMIDFFKSSIQKHKDWKNFGFRFSPTYTYDSIDAFYREVEAQGYNITFTEIDESIIDRFVEEGKLYLFRIHNKDFSPFSKGRPNLHTLYWKAIFSPENLKNVIYKLNGQAEVFYRRKSIQPEQMVIHKSGEAIDNKNPLTGKKQSTFHYDLIKDKRYTVDKFQFHVPITLNFKASESNIINYEVLQYLRTQPDVHIIGLDRGERNLIYLSLINQKGELLYQEPLNALKSDKFNVVTNYQELLARREKERDMARKSWGTIENIKELKEGYLSGVVNKIATLMVKHNAILVMEDLNTGFKRGRFKIERQVYQKLEKMLIDKLNYLVFKDKSEDEPGGLFKALQLTNKFKSFKEIGKQNGFIFYVPAWNTSKIDPTTGFVNLFYIHYENMEKARAFFGKFQSISYNHDKGYFEFAFDYNDFTTKAEGCRTNWTVCTHGDRIRAFRNPEKNHQWDYKTIHITEEFMALFRKYGIEFSGNKDLREIITGQKDAAFYKSLLDLFRLTLQMRNTNQDWDYLISPVLNENGEFYDSRKADNHLPQDADANGAFHIAKKGLMWLKQIQQFEGDNWKKLDLDKTNKGWLRFAQGYQF

SEQ ID NO: 30 - protein, 642 aa, synthetic construct
MSIITIHCRLIASEPIRRHLWQLMSRSNTPLINDLLKQVSHHADFETWQTRGTVPSNAIRDLCEPLKEVYPGQPARFYASAILMVTYTYESWLALQQTRRRRLHGKQRWLNVVKSDAELLGLSGSTLESIRQRAQDILSQLSTEMETQSAPNPKKRSQRQGQTHSSNDASLMPRLFTAYDTADDILSQCAIVHLIKNGCKISETEEDSEKFAHRIHRKQKEIDQIEAQLQARLPKGRDLTGDVFLETLAIATQQIPETVIQAREWQAKLLSRPASLPYPMIYGSSTDVRWGKTANDRIAVSFNGIDKYLKEADPEIQEWFKLHKEYPFRVYCDQRQLPFFQRFLEDWQAYQANKDTYPAGLLTLSSATLAWREGEGKGEPWEANHLALYCAFDTRLMSAEGTLEIQQEKSTKALKNLTHDNPDPRNQSTLNRLKNVPDRPSRKPYRGNPEILVGLSIGLADPVTAAVVNGRTGEVLTYRSPRTLLAEHYHLLNRYRQQQQQNALQRHRNQKRGVTYQPSESELGQYVDRLLANSIIQLAQTHQAGSIVIPSLTHVRELLASEITAKAERKSRIVEVQDKYAKEYRIAIHRWSYNRLINAVCSKAQQFGMTVESGFQPLQGNPQEQAKDIAIAAYYARTIAIK

SEQ ID NO: 31 - protein, 644 aa, synthetic construct
MSFVTIQCRLVVSESIRCQLWNLMANKNTPLVNELLKWVSQHNDFEAWQRSGTVPVKTVRELCNPFKTDSRFEGQPGRFYTSASLMVTYTYKSWLALQRKGRQRLDGKLRWLNIVKSDAELVQSSGCELDTIRTKAREMLAHLNAQGSPEQPRSTKQKRKKKKHSSVADPNLMGLLFQAYEATENALSRCAIAHLLKNDCQVSAQEEDPEGFAKRIHRKKKEIERLQAQLVSRLPKGRDLTGEGFLETLAIAVGKVPDNEIEQLLWRAKLLAKPAAMPYPILFGSQDDLRWSTNEKGRICVAFNGLDKAIPELKHNPFQIYCDRRQLPFFQRFLEDWQTYQANKDTYPLGLFLIQTGLLGWEEGKSNGEPWNVNRLTLHCTMDTRLLTAEGTEQLRQAEIIRLTEKLSAAQNPETLTETQQVSLKRQHSTLARLQHPPQRHSKPLYQGCPEILVGIGVGMAHPATAAVVNVRTGKVLVYRSTRQLLGNKNYRLLNRQRQQQQIHALQRHKNQIKGRTSQLSESELGQYVDRLLAKSIIELAQQFQASCIVLPHTNNLREHLAAEINARAEQKSDSKEVQNKYAKQYRISIHRWSYDRLLSTIRNQAEKAGISIEISSQPLRGTPQKKARDLALATYHFRQASSN

SEQ ID NO: 32 - protein, 658 aa, synthetic construct
MGIITIHCHLGTIEPIRRLLWQAMVESNTPLISTLLRQVANHSDFDTWQIKGSVPVKAVRTIGDPLKAHYPPQPGRFYASAYQMVSYTYESWLALQKKTKFSLEGKRRWLSIVKSDAELLELTGLSLESLRQSAREVLSQISAEIAAERVPDTQKNKPKVKSRKSKKKSTGKDKDLIGKLFKVYGTTDDLTQRCILAYLIKNGGTILDQEETTEAFARRVHRKQKEIARLENRLEARLPKGRDLTGDIFTDTLLLAQQQEPEDIAQMRDWQAKLLMRPADLPYPIRYDSSTDMMWKPDDQGRISVNFNGLDKFLKNSDPEVRSWLKEHQGYPFRIQCDQRQLPCFQRFLADWQAYTADAENYPAGLLTLSSAMLAWRKGKKNGKGEPWNIHQLALYCSFDTRLLTAEGTVEVQQEKIKKAQKQAKSADGKKLDEKQLQARTSNATTLRKLDNLPNRPGCKPYQARPELLLGISIGLSEPVTVAIVDAATHQVLTYRTSRTLLGEQHRLLRRQRQKQQQNRLKRQQNQKQGIRHQPSESELGQYVDCLLAKAITQLALTNQVSSIVLPDLLNRRDILDSEIQAKAERKCPGSISAQEKYAKDFRRSLHSWDYRRLIEAIRSSACKHGIPLEETFLTASSDPKEQAKEIAIAAYQARTED

SEQ ID NO: 33 - protein, 708 aa, synthetic construct
MNHKSTVKASVRHLTRHALQKPGEESDPGETTKTLKFRVDAKGHTRTRLDQIFTAQRQFVRDLLNKLEGMWDKDPQRFIRMVESSASDPFESKTSCYGWMMTKFLTGSDLPEHLSRSAATASVATLSGNMKSFVTRRENVLKDMAVVIERNMENCEAGLKDLCAELKIDRFKSPPSVDLESINAKTVEKYNEWVAAARVWCNLILAQKHKIPRGDAFFPTRLKAYPGFPGSVIHKEATYFATAIKELRKCLADLKQSHQKLFGALSNEEWSLVLERFPAPEGTGGKGMRHQLGKTLAVLGKKHPEWKPRQYIEELVAGLERGADSMAKHLENRQFSDRPAAIKLINLLNNACVVLMEPLRVKSLYKEYFEQDTPRRNAFGQARGSLAEPTDQADSLQILGFSLMDEVRGICRYDGFLAFAGDKEKDQWAFFFDPDGPGGMGIADPVKHPKGSTDSGYIGFGTMGGSRKRAQAAEKRLVRERLWIRRDRKPLQLPLHFGTRQGREYLWNFDHGLKKSSDPWMLTNGRLLRIYPHNRPQFSDYFLTITLQKDTPPLAISSGEKLVGVDRGEQNPASFAVVDAKGRLLERGNVGEKYTAKIRQFAEMKSDLQRTAGGYTRKLRAKERNTASALGGLVMRELLDLLVRHPGALVFERLSSGITTRGGRGTLMGNMHYERILSGLRASWPRQVATRHPVPPSTAKESRRSCDL

SEQ ID NO: 34 - protein, 709 aa, synthetic construct
MVLSTEQQQLINTWLKYSPHIWNLGLEQIQLLERNYARNPKIKIKKEAISEEKKEPVDRSPKSREQEAEQRQEDLLKQCMFPVSKLPWRCRYLHLDPRTGTPVGASGSSIKATAYDGKCWFVDGEWVEVKGKPVRLLIPYSQIVFSTKKTREQMVRNCTTPRGCDRAQKEGVPYVYTVSDPEDRGHYRRTVQGWIGNCYSLPWQRDRPIPTTPRIPDAMGFYPLEWCEGDREEPIVWTEGDQTKGGLSKVVKYLVELLEKKGLPKEIADIPNTFQYGIIKRLSTGWDNWKSYRFGKSKGVVYGRPRFRRTDTSDNTLSPKPGPGNHRLRIVGRDRCSIKELGDFAVPTLSDRWVNCEDGKPIIPEIRTYNIVRRPSGYYLQLCGDIQRSPRLLKKPKPVTAFDPGIRSYLTFEDGDRVENPKFYERIARRVAKLQQEISRKQTHNLILWLNQRHRTPDELVSLCPGLGWEAATKAIACKSEDAIVKAIGIYFFGHLREKVGNSKSIDKLRQQVSKLHEKAKMQRRHFIQKQTTWLARKNATIVMEDGMQSPVLKKRNQPKATGDRAGFEKTGQQQKTEITKKLGDTGYGEFASQLERKAAEWGRVAVRFESKNTSEECPVCGHLEDMPLHRRKFDCSKCGYSEERDRKSAILMLVKSVELGTVAEGQLPSNILKILEARRNFREPIGVQNEVIPNKRKRRQRKRSDSQP

SEQ ID NO: 35 - protein, 722 aa, synthetic construct
MADLNTPLINELLHQVNQHPEFETWRQKGKHPNSVVKELCQPLRTDPRFIDQPGRFYDSAIATVNYIYKSWLALMKRLQFQLEGKIRWLGMLKSDAELVEASGVTLESLRAKATEILAQFTLQPDTAEPQSGKEKKRKRTKKSKKLDGESSISDTFQSNTVEEPQPRKEKKRKKTKKSDGERSISDTLFEVYRGTEDNLTRCAISYLLKNGCKISQKDENAEEFANRRRKLEIQIERLIEQLEARTPKGRDLNDAKWLESLLLATHNVPENEAKAKLWQDSLLKKSSKLPFPIGYGSNGDMTWFWQFSLYNVPINLRFFWLWIYIDYLIAILFLRDALKNEKEWLHNLRINNISLLIKLWDLTVDVNCLASILFLHESFHNKYKRRICVKFNGLGEHTFKVYCDFRDLHWFYRFLEEQTIKKHYKNKYTTSLFALRTGRLCWQENEGKGKAWNVNRLILYCCVDTRLWTLEGTNEFKKEKAEEIAKSITKTKAKGELNEKQLDSIQRGNTTLANIDNPFPRPSKPLYKGQSHILVGVSFGLENPATVAVVNGSIGKVITYRNIKQLLGNNYRLLNRQRQQKHTLSHKRQIAQKIAAPNQVGESELGQYVDRLIAQEIVAIAQKYKAGSIVLPKLGDMREQIQSEVQTKAEQKSDLIEVQKKYAKQYRVSVHQWSYGRLIANIQSQAAKAGILIEESKQPIRGSPQEKAKELAIAAYHSREIN

SEQ ID NO: 36 - protein, 753 aa, synthetic construct
MMQALLGKTAAIPKQKDLKEAAVQLMPDFDETGTRRILLNFPLALDSAHIQGHLKKAELWNKQFQSWKNGDQLPFLRWPSEFDGKESHHWWEETKSFRVLAADLGTHHAASVALIEATQVKHPDSRFIGETSNQSWFARFLTGKVLRLPGEDVQVFRKASPLDGDKQGTTFREELYGAKGRMSSETECRHTIELLNALDQSSLLFDANSTETLKRQFSFPEQNDKILVALRRTQNWIATCVSWHWKLTQPDSDAQREVAISQIAEQDRKPEWKALATTRGESIGLLRDLLRDHITHQRQLVMTRLLETTQRILPLKGRTWTWQVHPEKPDCHLLSCTTKSNTSAKTLLTGQRGLSIARIEQLSELRRRWQSLNQALRRELGQRPLSAAEMRNDPIPDPCPDILRKLENLREQRVNQTAHLIIAQALGLKLKMPELSQDVRRKVDMHGEYEVGRIPADMIVLEDLSRYLSDQGRAKSENTRLMKWCHRAITEKVKLLAEPFGIPVLETPAAYSSRFCSLTGMAGFRATEVSWKDRHKFRWKALLKEEEESRAKAKVPSAMAQYAAGLFKDLRKIAESPMPTRTLLAPQTGGPIFVTAKPVAHPAPAKTRRSGTGAAVLPMQADINAAINLALRAIAHPACADLHHRLRTERKVAAKTKQEIYSTREIRRFSTEPATVEVKQGHSLPKERNSNLFFDPYNVAAFGRSRLSCDKANEYPYAAGPGLWKVVNNPEFQWCRCLEINRQRLTENEDIPW

SEQ ID NO: 37 - protein, 787 aa, synthetic construct
MRKTALAQLQTELKGKFGDPDFYLWLAKDENMDIWKHDSNPVFEIAQLNALKRLLAKRKQQSIYTQPDAVKHPKWSQFEAPGGTNLKNYKLIQDNEGLLLLELPLLSHSPNGLLEKNYTIRLAPSGQINNPEIVNGDISKLTFFYGDEQYAADLGGSDILFNRSFLEHRPIESLRQGNWGGGKHNTLAVWFKCVLDVEPKPAKPDWLKKQNDRYMLITPAAINHFKSGLLNKKHKELLESGLRVLTVDLGVRTFASCSVFELVKGKPERGLYWHADVDKDLWARHERSFTLAMPGDNVTAEAKDARIHAYEELNRLKQGKNFIRNLLRLSVIEDIQKRTEEFELFCAGREKFSGKDLAYKLSADDIKTLTSCLGKPIESWRVQIKAVFVKYEKIIADDISKWRTSTRPKTTERQYEMGKSYWGIEYLEAVRDFLKGWSTHAREYGQITRWDREKNGIFANNLLEHINNKKEDRIKTGSDLIIQSARGKLYNEKTKEWEDKFQPCRLILFEDLAMYRFRTDRPKRENTQLMRWSHRAITNEANQQASIYGIHVDTTGAGFSSKFYARNGCPGVRATKLTAQDIEYIYANEYVKDRLLVDGFGEHFLREGTIVPWMGGELFVSFSQRGKLEILHADINAAQNLQRRFWTRYIDVFRVPVVQIDKSGEQWIPQSNGERLKGGLYILVGSKAPVKFVKAEGDGFKVERLTQKTPKVFSDTEDSGLDEFSEELLEQGIDLMSEEGKGKSVFFRDASGIILRSDRFYESREFWDRVHMVINHALKEKYEPNPF

SEQ ID NO: 38 - protein, 1470 aa, synthetic construct
MVKQAAVTQRAYTLRLRGVDPQDQSWRDALWATHEAVNKGAKAFGDWLLTLRGGLDHALADAKVKGGKGKPDRDPTDEERKGRRILLALSWLSVESEKGAPEEYLVPHDLDKQSGTRSNWKTVDALREILRGRRLDESEIESWVRDCSASLSAAIRDDAVWVNRSKAFDDAVESVSSSLTRDEVWDMLERFFGSREAYLAPAKGAEDESAEVEQEDKAKDLVQKAGQWLSSRFGTGKGADFSRMATVYKEIANWAGKAQPGMTGDKAIADLAGALSEFSPTSNDLRGVLSLISGPGYKSATRNFLSQLATRTVVAESDLQSLQKSAKHDAQKCTANTGAKGQRPYSRVVLEQVESACGFTYLQDGGPARHSEFAVMLDHAARRVSLAHTWLKRAEAERRRFEEDAGRMGNVPASVKQWLDSFCADRSEASGALEPYRIRRRAVDGWKEVVAAWSKADCKTAEDRIAAARALQDDPEIQKFGDIQLFEVLADDEALCVWHKDGDSAKAPDPQPLIDYVLATDAEFKKRQFKVPAYRHPDALLHPVFCDFGNSRWDISFAVHEAGKSTVRKKKGQWQNSDPHALSMTLWTGSTVEPRRLRWQSKRLTRDLAFKEGKQNGNAPTVGRADRLGRAAAGVPEHGAVSIAGIFELEDWNGRLQAPRAQLNAIAARVARHDWDAKAKQMRDHLRWLITFSAKLQPSGPWLEYAAAFPDDAPAKPFVSRKGDYAVRHQGNDAREGHAKLILSRLPGLRVLSVDLGHRYAAACAVWETLSAKQVKAACQAAGHEPPKDSDVYLHLTKKVKKQKKGKEVEVEETTIYRRIGADTLPDGKPHPAPWARLDRQFLIKLQGEEEGAREASNQEIWAVHQMETVLGLSVPFIDRLIAAGWGCTEKQKSRLEELKKIGWTPAPKAESSGVQKENEEVDVCKPSLAVDELMSGAVRTVRLALQRHGRRARIAFNLTASKKLLSGGREEGLTDEGRVELLADTLADWYALFTGKGWTDEWARQQWETHVTPLLQGAALPKIPEDAEAAPKARKEYRADLTEHLKPVAETLAKDESLCARLHCLWATRWRENDVALRKRLRWLRDWILPRGRKAEADRAIRRVGGLSLTRIATIKSLYQVQKAFHMRPEPEDLRKNIPSKGDDALLDFGRSILDVMEHLREQRVKQLASRIAEAALGIGRMKRFAGGKDPKRPRERVDRPCHAVVIENLTHYRPDETRTRRENRQLMSWSSSKVKKYLAEACQLNGLHLREVSASYTSRQDSRTGAPGVRCQDVPVKEFMQSPFWRKQVAQAEKKQAKGKGDARERFLCDLNAKWKGRPEADWEKAGAVRVPLKGGEIFVSADEKSPAAKGLQADLNAAANIGLRALTDPDWPGKWWYVPCDPTNFKPAKDKVAGSPAICLDKPLKDTDGKAGARSREVVNLWRDVSSQPISANDDWREFTPYWNDAEARVVNILRQSAGLARDQGER

SEQ ID NO: 39 - protein, 1127 aa, synthetic construct
MPVRSIKLKLMTHSGDNAAMLRKALWQTHKLHNEGVAYYMKLLIMLRQRDVYKQDLKEPAESFRLDLLAHVRQQQQRNGSAHIVIPEMELTAALRELYELLVPSHNGGKGDSQQLSRAFLSPLTDPNSQGLEGTAKAGAKPRWQKMQAEGHPDWEAEKERAEREKEADPARKIMKQLREWGLKPLFSLFTNEQTHIQWAPAKKDRDFVRTWDRDMFQQALERLLSWEQWNHKVIEERALVIARREAYESEHFASEAQQAWLQRMRGYELERAAKLAEQSLGLTGPFRIKSRQLRGWKRVHDLWRKLDKKGQRSLDHCWEAVAKIQGAQPKDFGDPELYRFLAEPANHDLWLEEPERLFHLVTYNALLEKEELSKQQTTLTLPDAVEHPLWVRFDPKGGNLHSYELQSEDDKSPQKHLSVTFARLIWPDSTGDGTPLWIESGPIQVGLATSAQFYRQFRLLPMGDDNRLGKALEVQDYSAADTKTTGLLGGAKLQFERPYVEKHWQTVSEGDIGRAYFNVTIDMEPIQPMKNDRLISGLGKAFKTFKRDFMEVTQYKPADLEAWMATQPAKTELTGTKALQEGLRVISVDLGQRTAAAVSVFEVVRGKPDADKLFYPIRDTESLYAVHRRSHLLQLPGEAVTPEAVSERQSRNRAMYGIRMQVRLLSGILRLYGKEEAASRRKSITRTMDMIRSTDNLSMDDKTRWSSVYQRLADAAELDVPLWQSTVITAHRELEAIVSQAFSTWRTSSRHRGRGVGGKSMWSLEELDQTRKLLISWSKRAREPHAINRLDRNERFAQRLLTHLQRLKADRLKETANLIIMAALGYKYDSPEKGWLPVFPACQLILFEDLSRYRFKMDRSRSENSKLMNWAHRSIPREVYMQGQVFGLMVGHVPSEFTSRFHARTGAPGIRCHVVTQADLDGRTAMLKLVDQDFLAEVRDELQPGDLLPFRGGEIFVSLNGSTEKERALNRLHADINAAQNLQRRFWSRYPQDPIRISAQRITADQEIAYEPKTTKMKESMGKGFFQEFEISKSHHVYKWNSSGKLGGKKRIADSKDMEETDDLAEEAAAAEELMDGYVTLFRDPSGYYRASDLWVPQVDFWGMVTRRLESLLKAAINERPGRALSR

SEQ ID NO: 40 - protein, 1149 aa, synthetic construct
MPTRTINLKLILGRRANTAELRRALWTTHYEINKAVERIERLLLFCRGAAYRNFDHDGNEIAIAESEVVANALTVAREAQTRNGTKKIVTDEDVLIALRELYEQIVPSCLLDEKGNPMKGDAQSSNAWVSPLMDPESEGGLSVFSKVLDPPPEWVRMKAADQTGWEEKSKEWLGSADGRRLQSTPGSPPGWVRKLRNGQPWQDSFIVDQAKKREALAQGNAPVIKRLKELGLLPLAEPELRRKLKPNENGVSVWDRLAMRLAVAHLLSWESWNHSTKKAHDEAKATRDALQEEYRRYEKQFRELREYETERHERLKLEAFADDDRPFKTGIRAIRAWSRVREEWLRNGKAAQDRKQIVSHLQTKLRGKFGDPDLFLWLAEEGRDHLWNQSDTLTSLVTLNEAQRLLEKRKPYALMTFADARFHPRWAMFEGPGGSNLRNYDIESRNGELSLQLVLLQETETGGLVEKTFNVCLAPSGQLCHLAVAREGKKLLLKYRSGHQEFQGVPGGAEILFHRPYLEHAQRTDELIAQGAPGPVWCKFTVDVQPKAPKDWLDRRGKVAIPLEVRHFGTALSNQSNYAGKLETGLRVLSVDLGLRTFASCSVFELVKGRPTEGLFFPAADGKDTRDPEKLWAKHERSFKLCLPGETPTKLHGATRAAARDELRALKRDISRLRDILRLGIRVDNVERDEGVRAFVESLENGLANSALDSSILESLNDPKFRSTPELWQFCCQQVYDAAEKRVAQRFGEWRGRTRPKSMSWADWRGRRCYAGGKSIWMLEYLEAVRKLIMSWNLRGRSYGKVNRQDRKAYGTIASRLLHHINNLKEDRVKSGADFIIQAARGYIPVKDGVGWVKKFAPCRVILFEDLARYRFRLDRPRRENSQLMKWNHREILTEAAMQAEIYGMVVVTTSAGFSSRYAASSGAPGMRCRYLRRDDFEDGRPKPYVVAELEWMLGNSRSNEVAETQNALKENIQPGTWVPWNGGEMFVTLKPNGKPHMIHADINAAQNLQRRFWNRCGDAFRITCTADTIDGVQCYKLDREPAPRLLGALQQMEGGGGPFYLKDETGGQRYVMTPFAGKTGRTPPVREDVSIEDALEEALVELDEDSGGARETFFRDPSGILFNTRYWIPSKLFWSHVRARVWSVLRNR

SEQ ID NO: 41 - protein, 1163 aa, synthetic construct
MATKTVNLKMVLDRSDKPEAKELRQALWTTHEQVNKAVAEIERILLLCRGRAYWIKDKDKDGKDIDIEFKAEDVPKRALAYIKKHKKINTNNDAEIIKALQQLYEILVPSVLLDKKGEPLKGDSQSVGNSFARPLMRPDPKEGPFPDIAQKILPKSCESWIEEIDKEKYIKNNTDYGKCKIQNTEIEYFFKINNKAAQQWAKNEEGQAFIESMNHLSGAKNRWFKVYKDNDSAWPMLLAENHFEMKQEPRIFVREILWKKLSLLPLISMPFADFKWNSLALRLAVAHLLTWESWNHRCAKEYAKLSGRVNEKETTVRQIDANIIHKIEKYEALRQQELKKNSLANDGNEFKINPRMLRALDHVKEAWSKKNCATSEARKEVLAKLQTELRGKFGDPDFYLWLANDNNTDIWRHEDNPLFEVAQLNALKRLLKRRKKQSLFTQPDAVRHPKWAQFEAQGGTNLKNYKLIQCADGSLSLKLPLLSQTDDGIREKEYTIGLARSGQINDPAIINGDNSKLTFFYADEQYTADMGGSDILFDRPFLEHREIESLRNGEWGEKDGALRVWFKLVLDIEPKPAKPEWLKKQNDRYILVTPPAINHFKSSLLNKKHKDIIEAGLRVLTVDLGIRTFASCSVFELVKGRPEKGLYWLADKEKDLWARHERSFILSMPGDDASIQAQTARYQAYEELSRLKQGKNFIRNLLRLSIIEDIEKRTKELDLFRSGKEKFTGKDLVYKLSSDEIAVLTSHLQRPLEVWKVQIKAIFDKYEEILSNDISNWRITTRPKTSDRQYEMGKSYWGIEYLDDVRDFLKGWSTHAREYGQINRWDRERQGKFANNLLNHINNKKEDRIKTGADLIIQSARGKVYNKKKNKWEDKFQPCRMILFEDLAMYRFRTDRPRRENSQLMRWSHRAIYNESIQQAAIYGIHIDTTGAGFSSKFYARNGCPGIRAKKLTRADLNYINNNEHIKEYLGKDGFCGNLLKEGNIIPWQGGEYFVGFSTKGKLEILHADINAAQNLQRRFWTRYIDTFRLPVTQIDKDGNEWLLQSCGERIKGGLSLLVGVNTPIKFVKAEDGSFKIEKITGKKPKAIDENEDTGLDEFAEELMEQGIEIAGEEGKGKAVFFRDASGLILRNDRFYDSKEFWGRVHRTINKALKEKYEEPNPF

SEQ ID NO: 42 - protein, 1164 aa, synthetic construct
MPTRTINLKLVLGKSESTLALRRALWTTHAELNVAVQQIERMLLLCRQDQYWTTDGGDEPAHVAADKCTAEALSMGRAAQKQNGSEGKGTDREVLKALRVLYEKLVPSCLVDENGSPLTGDAQASGAWVSPMMDPESEGGLSVYAKVLDPPPKWVHLKEQEEPGWKEASAAWLQSVEALPLLSATGSPPAWVRRLRNGQDWQDAFIEDQEKKRREIERGNAPVIRELKRLGLLPLVLPHFRMKLNSGGKGVSVWDRLAMRLAIAHLLSWESWNHNTKAEHNAAAAAYNALIEEHVHLKSVFDVLREYERARHERLKEVALASDDRPFRIGSRSIRAWDQIRQDWLQKGKDAASRMEILAQLQSRLRGGFGDPDFFRWLAAEGREHLWREDDAVVPFVKINMAHLLLSRRQEFSLMTFADSRLHPRWIMYEAPGGSNLRNYALETAGEKLLLRIKLLNRNDNGGLTEEEHLCRLAPSGQLSDLTLSRDGKKMLLQYRSAHQDFEGEAGGAEILFDRPYIEEPRRKEEQLESGRIGPVWFKLTLDVHTKAPEEWLDGRGRIQTPPEVHHFNTALSNKSKYRDKIRAGLRVLSVDLGLRSFAACSVFELVEGRPEKGLAFPAADGREENDPRKLWALHERSFTLRLPGEATTTREESARREAAGQVRALRRDMRLLRRMLQLGAEDNDQERDAQLGALLEAMPAGDGVADDDVPGFRREDYEGLRDTSFRSTPELWMQRCQQFHAMAEKQLSQRFGEWRRATRGKSSSWQERREKRAYNGGKSVWAIEYLDSVRRLIMSWSLHGRKFGEINRLNRAEHGTVAAGLLRHINKLKEDRTKTGADLLVQAARGYLTNPDGPGWQQRFAPCRLILFEDLARYRFRVDRPRRENSQLMKWNHREISAETTMQAELYGIITETTAAGFSSRFHASSGAPGVRCRVLTAEDFADELPRPYLAAELGWMLGPSFHGNAEKAREALRWVIRPGMMVPWRGGELFASFGADEKLRVIHADINAAQNLQRRFWTRCGEASRITCLAVGEDGGYHLDHTPGARLLGALQQLENGKGEFHLTPADDGVSYVMTRAGRKKGTKAAEGKARAEDGELDELLADMPEEAGTRRETFFRDPSGILFESNRWIPSKVFWSSTRDRIWQEMQKVSLERQSSDELRL

SEQ ID NO: 43 - protein, 1193 aa, synthetic construct
MPTRTINLKLVLGKKEATTELRRALWTTHWRINQAVGRIERILLLCRGASYRTIGVNGEEKEVTQSEVEHQALQMAREAQKRNKSIVGSDEEVLKALRILYEQIVPSCVLDDKGNPLKGDAQSIGSGYAGPLLDPDTCKFEEDDEKATCGPFAETASKQFSKLPDWVIEVSKNDFCNDDKESFLRFKNASGVETFYRIDIDAATSWYISDEMQDTIKNNKAFNKDGWKKKKEKNDTSWAVDFAHKQLELADDPRITIRKELWEKLGLLPLDNLYFNKNEVGNLWNRLAMRLAVAHLLSWESWNHSTKKEYEKAKKKFDDLAYEYAGLKDKLKILHEYEKARHQTLKLVAFADDDRQYKIGARAIRAWDSVRTQWQKEGNTVDTRKKILQDIQTKKRGKFGDPDLFIWLAQDGHEGLWKDDDALTPLVKLNVSQRLLEKRKAYSLMTFADARNHPRWAMFEAPGGSNLRNYELNTDNKGLCLVLPLLQDVENDGLAEKGFTIRLAPSGQLSNVEIMKNDKKVALKYRSAHQDFKGALGGAEVLFDRHYLEHTGRSDMAIEYGEVGPVWFKLTLDVQSKAPNDWLDGKGKVATPSEVYHFRTALSNKSKHEAKLQPGLRVLSVDLGLRTFASCSVFELVEGKPKNGLFFPAADGRDENAVNKLWARHERSFKLALPGEDPTKQEKIARAAAMSEIRTLRLDIRRLKDILRLGIVEDEEKRNDLIKALEESLSSETADSALTLDDLSGLDDKKYRSTQELWQQRCQQYYNEAEKKISERFSQWRTHTKPKSSSWQDWRDRRSYHGGKSIWMIEYLDSIRKLIMNWNLRGRSYGQVNRQDKKQFGTIASHLLHHINNLKEDRTKSGADMIIQAARGYIPSKDGVGWIEKFEPCRVILFEELARYRFKVDRPRRENSQLMKWNHREIVSEAKMQAEIYGMIIETTAAGFSSRYLASSGAPGVRSRYLNDEDFSGGLPKDYLVYELEWMIDHSSSMSKEEKKEVLHKKIMPGMWVPWTGGEMFASMKDNKSINFIHADINAAQNLQRRFWNRCRDAYRITCRQVDHENGDRYSLENQPGPRLLGALQQLENGDKEFYLEMEADNNHFIMVEAGKKKTRASEDEQDVGEDDFAEALAEIEENDESGGKRETFYRDPSGMIFDSRHWIPSKHYWFEVKKRVWEKMEKPLILYQEKDDGLQF

SEQ ID NO: 44 - protein, 1197 aa, synthetic construct
MFKQEITKTLSAALLPYGQTDVSSLQEVIDRDKKRHKYRKDIIKMIDERHKALLSKIKDAPLEFHRLEELLRKRNKDKKDTAARKELEAEQKALCQILIDWLSSNNKTEFAYLTASTPAKIVSEMFAKNPENKKLEVFDKFCTYLSDYSAARNTIYGTANKAGSVGCRLVTENFPIFIKNKDVYAKLSEEYPEILKTVAKNLPEQSAYISAFASTDDYMMFSVQDGIKNYNAIVGETNKVINLYIQANPDTKTALKKLRMKTLYKQILFDDEALVAAAFPRIYSVKEFATLVHTIWEGVLDSGKVLDDMMVLLKNIRSYDTTGIFIKKNKIAFVSDKLYGEWNRIKDTIGETKDDYTALEALIFSDADFDNLFKDLNKFKSEAVLAYVEVNKFADDEMMSEDAVPHITELMYALNDFRKLVDIIAIPDDSVNSDPDFYNKYKEYSEKTAAIKSAYNTAYTFVTQRPEDISKKFRLTFDYSTFMKGWDIDNICNNNYGATMFCKDGRLFVGVTNKEKCPDFDDIHPANPGEETFRMLSYKKLGDSSRQIKHMFINDSTPDNIRKIVLSKKEKEAYEHSDMVKVIDYYKKCLLSRYSGIYDFNFSETSDYKTINDFFAEVDKFTYKVSFIDIPASQIDEWIAEKKLFMFIVHTKDYKVGAYGTKELFTIFFENLFTEDNLKNDVIRLAGGSSIVMRPKAVENPYRHKVGSILLNKRDKDGKPVPNKIYVSLSKYLNGRLKKKDLSRDALAYLDKVVWHKHQKEHIAVEDIVKDKRYTTDAFILHLPITINPGCRANEYEINEDARKNAKYTIGIDRGERNLLYVHVTDCKTKKVVEHRSLNEIDGYDYQLRLTQLEKERQDQQRNWRTQSQIKHLKKGYVGKAVHIICELMLKYENSIVVMEALPKEMMQRRGSAFGAVYSQFQQALLNKLSYLVVKDRKPLEKGGILNGYQFTIPPKTLEDKPRQYGSVLFVPPGYTSSIDPVTGFTNLFMYKKYDSSNKKAKNFTAKFDSIRINEDGIPELKFDYSNFKTFQKWPDRQWTCIIDGKRILRQKNQKTGVWEYEDVDVREAFNKLFGKYSKLEPGTDVKELIGKIRKTDADIDVKFVKLMKTVFRLRNSNPNTGEDYIISPVLDVHGKTFDSRDQRNLELGLPVNGDSNGARNIALKGAMTLHNAGVAGKLVAVGNSEWMQNVQRFSSE

SEQ ID NO: 45 - protein, 1256 aa, synthetic construct
MLSHFTNQYQLSKTLRFELKPVGETLKHIELKGLLAQDETRSQEYQEIKIIIDKYHKAFIEEALHDVSLSKLAEYETAFFDKNRDEKAFEKLQDALRKEIVMQFKKDAHYATLFKKELIKADLQNWQELSDAEKELVSHFDNFTTYFTGFHENRANMYTDEAKHSSIAYRIIHENLPIFLTNKKLFEIISQKAPQLAQETQDALLEHLNGAIVADMFELSYFNRLLSQSHIDLYNQMIGGVKKDELKIQGFNEKINLYRQANGLSKRDLPNLKPLHKQILSDRETLSWLPEAFENYEELVQGVQAYFNSEVLTFECCDGKVNLLEKLPELLNQIQDYDLSKIYFKNDVALTSASQAIFKDYRIIKEALWEVNKPQKSKDLAADEEKFFNKKNSYFSIEQIDDALKEAKLFASMMAYFQSESSKTIEQIQVAYAQWKQNTSNKELLKAFLEVLLSYQRLLKPLNAPNDLDKDVAFYVYFDTYFASLSGIVKLYDKVRNFMTRKPYSLEKFKLNFENKGDFLGGWVDSHTENSDNGTQAGGYLFRKKNHIGEYDYYLGISKDTKLFRSHLQNEIQEEDKSEFERLDYYQLKSASVYGNSYIGGSYDEDKKMLLKSIYKFAENNHPLKADFDKYIASQKGDNQPTPNGMINIVVEKYPQLMQELVKNEEFEAANKMVIERLKQTILSLHRVPKSQDYRDKVFSLFIEPIQIIEELSKEKSFFYFHVSDNEFKSALARDEKPLLLFKITNKDLSFADSFASGKRKSRGTENLHTLYFKALMSGNQNVYDIGTGEIFFRKKSIEYSEERLKKGHHFDDEKKLKDKFAYPIIKDRRFAFDKFQFHMSIIQNYMYPKQPKGFEEKVNLAIHDADNIKVIGIDRGERHLLYLSLIDSNGKIIFQETLNSIVNTFNNEPIDYHSKLDAKEKDRDKARKEWGVVENIKELKEGYLSHVVHKIATLIVEHGAVVAMEDLNFGFKRGRFKVEKQVYQKFEKALIDKLNYLVDKKKQPDELGGILNALQLTNKFQSFEKMGKQNGFLFYVPAWNTSKIDPVTGFVDLFDTRYASVEKSKAFFAKFKTIRYNEAKDYFEFAFDYNDFTDKAKDTRSEWTLCTYGERIVSFRNAEKNHQWDSKTLHLTTEFKNLFGSYTGDLKATILAHDDKEFFEKLLRLLRFTLQMRNSITGTDIDYLVSPVADEQGNFYDSRKADASLPKDADANGAYNIARKGLMLMRRIQKAEDPKKVNLAISNRDWLRCAQGLEK

SEQ ID NO: 46 - protein, 1276 aa, synthetic construct
MKTALFRVLFIINLNRRKMNQFSQFTNMFSLSKTLRFELKPVFDANQDAFMSLIEKDKEIDRLYREEMKSMMDDMHEVFINEALGGAELSVKLIDELELLLSERRKIKVAKKENKGVSYSKEEVDNNKKVDKVKGRLREEIVKAFNQRAKYWREEKYPHHKLKKDDVGILFEKEILKVLKDMNPSKVGIISNFDNFFTYFTGFNQSKENYYVVEDKDTAIGNRSIDENLMRFLENRSKFYEALKKINSLSKYEAYFQNKEYCKYLNEEGINAFNLIVGEINKEINLTTQQDKNLFVRVPKLKFLFKQIGCRDKECFILEIGEGEEWVYLNRLKDLQFGIVDKIKKGYEDFFRNIEAFDLESIYLNKASINTISSRWFLNWHRLAELLQAKKLIKAGELEKIPEKICLLDIKECLVGETNYEDIFRAGKITEQFSNGEYSQFFNEENAWEVFLRIWEMEITRNILAIEEKSKSLEVMSQESINCITKEERISFIKDFCDAFLSIERMIKYHKVKESEEKDGDFYEWVDYYLVNTGLNSYYNAFRNYLTKKPFKQDKIRLNLNSGNLADGFDLNKESDNLCVILRRSGVYYLAVISKNSNKVFDKVKNVELYEKDDQGWEKLEYKLLPGASKSIPKQSTQLNEVVKHFSSSNDDYVLEGGRYINPLRISREVFDLNNVAYDKEDIKKISLKKEDGVKKFQKSYLTLSNDLGAYRGALEKWIDFCKEYLKCYDSCAYFDYSHLKESSDYESIDMFYRDIDRASYSTKFVDINKDVLYEMCEKGQIYLFQIYNKDFAEKKKGKDNLETMILKNLFSSTLIKLNGGAGLFYREKSIDVEYDKERNKDKGFGIIKNKRYSEDKFFFHFSVTINFGTSSAKIEDFNKAINESLCQGDYHILGIDRGEKHLIYYSLIDSNGMIKKQGSFNIVGGKDYNQLLAERAEEMRQKRESWEEIGKIKDLKEGYLSQVIHEIYQLVVDNDAIIVLEDLNTQFKAKRTAKVEKSVYKKFELALAKKLSHLILKDKEPEEVGGVLHPYQLTPPVDAGMVDRFEKKKQWGVMFYVRPDYTSTTDPLTGWRKTRYISNSANHEEIVRNFDYRNKDYIKIFFDKDKDCFRFDCDGWSLFSFKEIERYYWSNKEKNASGNYGVSKKYLLDECFSDLFSDIDKTLDINGQILSAWEKKDQRWKTLVFYWNLLNQIRNTDRAKEGDENDFIQSPVWSEEIKGFFDSRCCYNKLLPNNGDANGAYNIARKGLLVLERVKENPVKPDLFIRNEDWDDFIR

SEQ ID NO: 47 - protein, 1322 aa, synthetic construct
MPLSRIYQGRATLLELLSSEGEWIEQPDFDAYPKSPLWRHHDIFQRAINYYLVVFARLANLGESQNRVIQDLPKRMESHWEQFPGGPKRSRGLRDSLAGWVPGIGSSTTFEDGLSTISLPEDLVPEHGLLALEALLLDLGKGGDGIVRNTSKDYLPFYCITTHGKNYPRSTAIMRKKEGEKILPSVLWTESLQDGRDRLSKLHMEYFANPDATGRLRKGANLRPLLEEALDEELAIGGIGQQLAGDLRKQIRSISDDQSMPAHIGSSVKGAKKKRLWRLYLLMSQVHPCSELFSLMREVQPRDKVDVLSIAQPDYESRLTKHGQDPVELARGRRGFVFPSFSSLPPWDKAEGGIPSWKDFDIIAFAQALTALNQFKQKTEERETKRNQLESRFQYMTGAVDKWVAPKTNAEEEPESAPRRIEITDLRLAQTLRTKLVEQGEAGFTKAALRGYRDIREAWLKLKQANPDVSRDDLRKVVTDLHREKGDQMGYVALFLELCEEQYHVLWRDEEPADGYARDIVWALSDHDQLVRNLARKQEVINLTPAEPEYSRRLFMFSDLTGKSKVLFGDETIEVSIASPKDGGGRIAAHRARIRYSSPRQLRDGLLGSETSLWLQPMLKALGLPPVAPDKEPAVALMPKVQDIGSTDCIPMLLNFPMTLSPVPLSGTERWKKRFNGTREKNLHLLWENGTPGDGKPFTILSCDLGLRMSSAWALLHCSMAEPDHERYRAFDADGKWFSELKKTGTHRLPGEGGRKKLSNGETITDARGRKATKVETAEAAALEVQLGAVEEDQPRIQEEMATKTFPEQNDRLLWRAGRLLTRLRTYHRWSCAVAHDTSTENLIAELEGYQNPELVKCRKQLSEKGDTHRATFADDMGQNFRELRSKLDRVLLVLAERCIPLRGADWMWQRNPDQPDSTSNYHRLIRQSRPGKDGTKRRGQRGLSIYRIEQMENLRKLFLRLNRAHERTPGETKPIGEDRRGERPGEPCNDLLEKIQRIKNERVNLTAHLILATALGLRLKPTRDAKTPDYVHGEYEKIPGRQPVDFLVMEDLSRYLTTQGRAPSENRQLMRWTHRAVLLKVKELCEPFGLPVVEVTPAYSSKFCSRTGMPGFRCREVASSKQLRGRLGRVWNKQKVEPQKLEADEKSLFDQITKLEEVNSGRKAAKKPLHTLFVPEVGGPLFLPAEGVAKPANADTNAAINLGLKAVASPHCFDIHRRIPSERVAGEIRARRKNKREQSAFDAKKRTIQLAGEPSSKLKSSKNPNFFYDPEAIADGDRAECEGLKLASQVALLSKVRSLELAQCLALNSIRLEQWQDNLPM

SEQ ID NO: 48 - protein, 1331 aa, synthetic construct
MNRIYQGRVSKVEIPDGKDEWKKLDDGESALWQHHQLFQDDVNYLLAAFAALVPTSCEDDIWKDYQAAIERSWESYTGRQGIWDRPFENACVIVGCKKDASFKEFRRKLNSLTGSKASEKQKFEALKQLFEPATEAAKKLKKHDEPVEESLKGKAKDLFGSTLVNLCAQKTKVTPRDVIAKQRNRASECTKKVNEGERLKWADVFYFKTDTSAAKWSREDAAKNIIQFLDKLLGEVEEKEKDAKTSDQKKKMADLAERLEKQKKPLAAWCNNSKTDLPTTEPTRKGSGGYDLKAAVLFSLQPDLDGFRDAFLLFNQARLKEEFATTEKGDAAYIARMAGGVARPVFPFFCDVWAGKVNDEKIGQGIWPDFEKQAFSEVFTKIGQFIVRGRKFELRLAIADQIIAKIETQKKSDARLQAVERIAEDLADELPDTAVDENGQKRPYGIRERTLKGWRKVRPAWREALKKTPNLTAEDLIKQKNRMQERQREKYGSASLFDRLAKEPEIWNHDDKEDAVETWADYVENLEEKAHLETERLFAPAHATLSPRFFRWSETNNKEHLEASSPDVPFELKADALDLSKKEKSQIKIHFWSPRLWRDGLRGKKENLDKDEPDQNWMPPVLRAFVKARKWPCDKQSFAGASVRLAPRCKENIQLVFEPELHTEILSAKWKENFPFSPAKNKESESVGLFWPRTKEDKVLWFDKGETRCLGVDLGLTNSAAWQILQATNKDATAKAPRLRHRLNPDSEKAAWFAHSITNGIVRVAGEDCWGWRKFAPDEKAKLRAELKKPAGKRNALCRKFLSLNREIEFETATHSFLPELSGSGGRNPTDDETKEAAEFFSTLKTKGFDITDRQPSWGKNLSFPKQNDELLWGLKRVRAQLFRLNRWSEQLGKERDSKPYQSAIEIIGNLRSDDPLIELATLKSEPKRLKSRIAELAGEYLDCFKTLLPRIADRILPWRRGHWSWKPCDNDWHRMELDASKPRPEALLAGQRGISLPRLNQLKDLRQLAQSLNHLCRRKQIKRNETVPEPFEDCRQAMEDAREDRAKKIAHEVFAIALGVELAPPPPDKQERKQTESLHGVYRCLERGPVNFIALENLGGYNPSAKQGRRENRQLSSWLKGRIHKILGELCEMVGMPIVLVNAEYTSRFSAKDHSPGFRAEEVQTDDSRRSFWQRKAKEEPSGWQNEFLCWLNKVPDGKSLLLPKKGGEFFVPLGEGTSLYHADLNAAYRIALRALAHRDRAELLGQTWIEKKPYLVDVAGVFPDSILRNGCAFKTISSSERLWEKVNGDLAMQRCREINLARFASWKIALPQQIISEALPPDEEDDIPM

SEQ ID NO: 49 - protein, 1331 aa, synthetic construct
MSLSRIYQGRVNRLELKSQDGDWVIQNEADRPIACPVWIHHATFQRAINYYLLAFARLAKLGKSENRVIQDLPSRIGSHWDSFPGAPLRSKGLRSSLKGWVPGISGNTDFDTGLQAINLPDDLDPAHGLLALESLLADLGGDGKIQKGSKTYFALFCEASSKANFPRSRVGLLKDEGKYLVPTLLAGDTADSAVLASIRERLQFEYFANPNESAEPLTDESSHKRLAEAIAGARKTEPALGDAVWMPIEQTLENLNEPPAIPRYFFGGAGPEIRKRRLDLYLLIKHLQAPRPIFDALRSCFSEPPKNAPQITDTEQHLRSLGDDPILLARSDRGFVFPAFTALAPWNTDDLGKPSWRNFDIMAFAQALTALNQFKLKTDEREDEKKKLQARFDFMTGVSEKFDAPKPSEDDEVERAPNRIRSSDLELAAELREKLVKVGEFGFTKAALRGYRDVRDAWLELEKQGNNPTQKELRTIVSEHHSQQGERFGSVPLFQALCEDQYRPLWRNDEVEDGYPNDIVWALSDLDQLVRDLERKQEIINLTPAEPEYSRRLYMFSDQTGGTKTIIGDGTVEVSLAAPLEGGGRFGPIRARLSYTAPRMLRDGLMGENGNGWLQPMFAALGLPPLKPDKDPACALMPKVEEISRSGTAPMLLNFPVTLSPKPLANVERWNVKGKKFFNGTNEKNLHMLWNDGAPSDGQPFTFLSCDLGVRMSSAWALMHCGREKPEHDRWRMVDSGDGQWIASWSKTGTHRLPGERGVISLPNGKKVHDSRGRRADGIETAAAVKMELRLGGVEESREDIQRKMNAKTLPEQNDRLIWRAGRRLSRLRTYHRWSCATVHDTSPENLRDELKGYSDSDLTPWKDRLDAGDNDARDAFAKAMGEKFESLRSELGRVLTELAERCVPLRGADWVWERNPDQPEPTANYHRLVRSARLGKEGTRRRGMRGLSMDRIGQLENLRKLFLRHNRSFDRGPGETKEIGTARKGERPGEPCRDLLNKIDRMKNERVNLTAHLILANALGLRLKAPTTDKDDYVHGEYERIPGRNPVDFIVMEDLSRYLTAQGRAPSENRQLMRWSHRAVLLKLKELCESFDLPVVEVVPAYSSKFCARSGVPGFRCREIASGEQLRGAQARLWRDWNDETKAKLAGEAGSLFRQIETLEKINKDRLESKKPPHNLFVPEIGGPLFMPASGDLLPSNADTNAAINLGLRTVANPKCFDIHRRIPSERNGQQLKARRNNKREKEAFDANQRTIEMVDEPSTNLAKSPRPNFFYDPEGISNTDRARCGDLNLTSQVALLGKVRASELTRCIALNEARLNRSGWRNDQDEIPM

SEQ ID NO: 50 - protein, 1365 aa, synthetic construct
MNRIYQGRVSKVELLPQNGLAPAPAPLEILGDHHQLFQDAINYYLVALAAMASDDESPLGQMRVRMADAWAPFSRNGILLDGLRASLLRAKVPGISPESSFEEGLIAVLAGNSVEASVLRLAAELLLSKVGGEAAIQQGGRGYWPRFCNPKANPTWDYSPDGLRSRAGKDKLAALIHSELTDIERNELAEELNLSWTVKLQPGNAYEGEKARARLKEAVSFFEKALANPDRRLAGLLAMHPDVAKHISALRPSIDALEKPIVPANRKAAPALVFATLLFKFFPSIETQSLLALHVAKPTANNKPETADRFASLGDDPIRIARGKRGYVFPAFTSLSIFGATGPDDIRWGEFDIAAFKEALKTVNQFRLKTEERREKRLKYEAAIAYMEGLHAKWEDPEGAEESEPPHRINGDPRIAGVKKLLEEQVKDLELTDADAPSITLSRRRLRGWTKLTEKFHQIVEPGEPFSTEKARKLKGALAKFQTEHRDEMGDARLFEALVTNRDYWCLWQRPTEAQRAERAAHDWSDNILATYRDYLGFDEEAKRLREPIRFTPADARESRRLFMFSDLSGRSKIKHRKDSLTVEVSVAVKTVGGIWQETRALLHYSAPRLRRDRLRGVEGADNLTEAPWIQPMMEALGLPEECTQDIADHAVALMPEPGKNGLRYLLNFPINVEPEKLMVLLGKQDLWRGQFNGTRDKSIHLHWPSTMDAKYHEKAWWRREKPFTCLAADLGTRACAAIALMEFLPGEPVGQERLIGYAEGKPWKARLLDNGRELLRLPGEDMKILEAGIWKEEPYGSKGRLPDPEETADFERWLKVFHIPVDDYLHAQIENLSFPEQNDRMLRIFRRAQGRNTELNRWLRFLREGDTSRVSEVLQEIGEKESRADWKKWASDKSIAPLMESLRLAILDHQELLKKEIVLLANRILPLRGRRWEWVQREHGKNWILRQTEDDIGEERPKIRGQRGLSIARIEQLEELRRRLQSLNRALQRAPGEKATMGRSARGMEAPDPCPALAEKLDSLKEQRINQTAHLILAKALGVRLKAHSLPDHERRLRDMHGEYEVIPGRKPVDFIVLEDLTRYRTSLDRGPSENSRLMKWCHRAITEKLKQLCEPYGIPVLETPAAYTSKFCSRTGLAGFRAREIGPGQTHFMGRTQRSGPEFAELQKMLDALESAGRTDLRLIVPARGGPRFVAAHQFHSQRPEELTDDADLNAACNLALRAVAAPDMHDLLPRLRCQTENGVLTFVGSVKNPKPSKRDAARFQKPVTFEVVNTSQHPTEHDEDEEDLRRTNIFVDLGKVALFEKVKATNWDTPLATSKGLWGTIRRLEFRYCNRLNRRRLEKWGIPLSDAVEGALTPDPDDEIPM

SEQ ID NO: 51 - protein, 1648 aa, synthetic construct
MAGELYRTYEVLVEESVVREADKDPKTKKETRYNPEVAAALRDTNETFQHAVCYYTLLLAGLVKDVKDPKDPADGRLLNPLWQHLRSGPMRQATDSLVRQLAERYERLAGVTDAEGFLERVFVRPEKDGPAKERMLEQARVQTYRLLEKQGTKLDKEGLPTECETLNVFASSWGSILCDPNNETEIPGNGVYDIIHRRLLKAEGNSEAVKRALDEAIITSEETHRGDFERELAHDIAKVKSDKAKAKKVAAKEQLFRKFVEDRRSQTRSNVCKALSDGLYSKPRDLGMSKEEHDLAEKELAALADEAKTTALDDPRLRRLRYGARDNSLEKPLFRLLWLRDSPQWAPLVLQDFCNYLEKEKPLPEPELDGGHLAEAPFANLTKNLFPLFVPYMLRIPSRERSAWWNFDVSAFAAAAEDVFKYKIRTQERKRRYDRFSQARDAYQGVVPKKLNVKQSHTGRPLTILGMGDDPRAAKMEKLLDDLASKLQFVGKYGLRPSTIGGWADLRRAFRRLERKAEKEKWADTRLEGELQDAVVKAQGDNPDGFGSAAFFEMLCETEFHCLWSARLPTPENRERHAADFVRYYVGYSEVLEEIQAPEETEWETKHMAPIRFTWAGTKNRHNKTSYRHFDFKHKLGAELKGVRLFRKLNRGADKFPAYELREEDTLTLAARRLKRDRVMTTDGNVVEALWCPPLALENIKDSQAAKQAPKIGKTKKGTKEAKAPEVSFSLMVSDPDEYETEPPVYLKVAIPITPTELNRTLRPNKLQWGGSLAWEKEGLDFTGRHLQWPVDLRVGQVGKARALVQDATSEELKSALERAATALANGDRRAAKLVQMEIKQLATSLPEAEAEVCKKAENYLGDAAKPLWCGDADKKIEQFQIGEQRRSEFHALSVDLNVRFAAAYARLRVFVNEGNTPPEVLALSRRLAPEGAHTTDIRARAYAHATLRLSGEDAHVWTKTGKGVFELRPEDYGKKGRLPVDDPTGGTEETKDFLALADALVPLKSLPVRSVETLSVPEMGDVLIRRLQRRLSRIKSLFKLRWRVLGLMERNPDTFNYDRPRKPEDQRRHRREVATLLGRLAFRQRQEDESEHPDNKRLRDMLNPSGDAWKTIGDRLDARKEESDEKLRCAELDTVIEEWDWPALGKEIQGQLDSLLSAGRGWENLDDVLRKKLAYLPDSERAAGWIELLALLVVEHCLPLRKRHWHWKGRRLKQGKEDEAPSHVPMIRGARGLSVARLEQVTQLRQAYQSLAKQLKRANDYPQVPGRPGIEPTPTERAESVDDCCEPLLDKRNQLREERVNQTAHMILAEALGMELEDPAKATWEGMPKKEAKSACDLHGRYRQKLGRDGQPLPRCDLIVLEDLSRYRTSQDRARSENSQLMNWCHRAVMEKLRDIGKPFGITIVTVGAAYSSRFHSLSGMPGVRVNHVSRESLAQMPYLAWAKQTNRTGKPSPRAVSIGELLKLFEENPGFHGELLIRVDGGKEFLPIPHTQEERATHNGLMNADENAAINIGLRALAHPDCLDIFTRIQTKAQDEQTLRVRNRRGWFAELAAEAEERSARCASKPNAEPESIAGAEPDEPDDDAVESAGFPNLFIIQDGTILLAEHELYRHERFLAARQGPYWGRVRDLCEGRIRELNRRMIQKP

SEQ ID NO: 52 - protein, 1389 aa, synthetic construct
MVWAHHVLFQDATNYYLAALLSLASEPDNPLFVIRQRLSPEDPELARDQIWKSFSRRGSKRKGFVESLGRHLGFDPHTATPDDVFQTILRDNEASAQIRDLALKELLAECNGEGAIQQKGREMLPRFCWSDFSGSYPHAATARAREENTLRLHTELHELDSPSALTAFAVEVDLGWVVNLDTSKPNLSEEDSRERLIKAVDHFAQAFGGRATTKLGERARGFLQSKPQSAANLQTLRERIAVLGANELPSIPRNRKSIPDRSEALLLFKYFPSPFTADLLRLSFPVLDAKKRQRASRNQVDNSFLRFGDDPIKLARGKRGFVFPAFTCLPSWKDAVPGQPAWKEFDIAAFKEALKAIHQIETKSAERKAEQAQLERLLDCMEGRAKWFTTDESEMPRPVLIACDPRIKQLEKIQQSLASSDDSLDEELREYGIRSRTIRGFRELATIWRQKTPPGTPKGTSHADQLKEILREHQTKNRDSMGSARLFEELTHPENWHVWLEPSPEEMQAWRNALPNSADRSLDFAPDPLTALARRLEIEREISELDRPVRLTPADPRYSRRQFYFSDVVSFSQRGNYRHDTAAMAVTVPIAVETDQGWIQQRVRLCYAAPRLKRDRLRAEDGSLGSTAGFHQPMMEALGVPPAVPKDLSKLAVALMPEGLPDESGRSRLFLNFPIEIDTAPIRRHIGKASAWDKQFASADGKNMYLRWPSLTSGLKSPPQKWWWEDRRSFTVLAVDLGQRFAAALARLEVTGDAPSREKPGRFIGRSGETSWHVGGLRTALVRLAGEDAQLVEKGQRRQEPFGSAGRPADAAEVEEAREICRRLAFDMDDRIGGANTQGSQLKRFPSFPELNDILLIAHRRGVARLRRYQRTLRTLHDDAKAGLIQYNELRASGDEDLPSIKDASSAKAHLEEACHRLRGTLSETLVDIAGRVAPSRDRRWIWEQAKKEGPANHCLVRSPPLPQRNRGVRLRGQRGLSMDRIEQLEELRRRAQALNRALLNDDPSKATIGGPLRGLDLPDACPEIGEKILRLKEERLNQTAHQIVAEALGLRLKPPTKSKAQRRDRDIHGEYEKVREPVDFIVLEDLSRYLSSQGRAPSENSRLMRWCHRAVLHKLIELAEPYGIPILQTNPAYSSRFCSRSGVAGFRAVEVTHASRYAFPWKHELAQYERVCSGDLKPEPHRRARLEMTHLLFAQLEKLEATRTGRPPRSLLAPQPGGPIFVPSRGPTTNADLNAAINLGLRAIAEPSVLDVHVRIVSAADNGTLRTLAKSRHAAARWGVKGRLIEMHKPEELARLLKESRQPNFFVDLGCDADFQRARVDGIGEPIASGKGLWGSLRQKEWRIINRLNNNRLEKKEWLGHRPLPEEPEPVSSSGSPWAEDDDIPYNE

SEQ ID NO: 53 - protein, 1817 aa, synthetic construct
MNTIHRSYEVLIEETVSAEERQRRKHGTSKESRFNLKTSGALELTNEMFQDAVCYYTLMLAGMVKDALWSEETFGYAKWMEDKLKTKGWQARQAQLTGRPINPLWNCLTNSVEMREATANVVKRLAEGYPHAIFHGAKDAGDLVQKAFCYQGEDRSTDGTRREKRRGQSPAQLALVKTFVQNLMAEAVNVSGKADALNDMASFAGVWSGHLCNPSGRSRPKGTGIYERLHTELRKLVNQSENGRRWIESEQALIAAVQAKDTPERRKQLRTAVEAAWVSVICDPVLLTKVCERVQELAESRAAENAKIDDSELRREITKALKSQDRKPTDTEEIIRERVKKEFDRKAKNAQETALGAFRRAFNQPRHAEKLKLTPHLSSLRQEIASAQIDDKRLQRLQERSKKDAGEFMEILCRLRWVSSRSDLRVLAASDALAFVIDNEPSESTEDKLPYQDSAGEPLFPFFTNSLGLCVQKQVVWDTFDTAAFKRAAEDVFKYTNRVAERRRKIEGLKTEKDLFDGKGRRSEKQKKDGKPRFAIFGMEGDDERRKRMEEVISELGKDRASGESGYGIRSSTIGGWTYLRPELLKTYEETKNKDKLEECLLDAVEKVRSQSGGRFGSADFFYALCREENHILWLDDWEYRKKQEWHAPDFVAHYARYSRKLEDLRDIAEENENWKPKNINYSWPGQLHFRKKERSFRPFDFKCDIRTDPKIDLFSRNDAGQIHRVTTNQAAKGKTLEFPLTISFRRLKRDRITDGAGNSVEAFFAPPKLAAQGSPQPPVLAGPRIRPDDIKAKELDAFLEDFTSDKMPEAAKLIQGLADELKQHHLEIQRLTKDSLTSKKSQKDLPQQRRTVVALLCNKLNRLISSKQALYAKEKLSRQEQKRVDEEPLGLEHRLQNRRAIQKTFGGFVKDVSQCYNEAVSILAPKRPGGAFHLMFAVELDEADLKDFRVPAIPAQQFQDAKQGETWVRAHLRWPVDLRTEAANRKRKGEKAKEVDEDVLTPEDDDPNAKGRPVLPEREELWCAPKNLEKPVHALFVDLGVRFAGAWSRARIRQAEGAKPETAHVISPPKGHSDYQGEIWCEFYNEGTFRLQGEDAKVWHRPKGGDYEKENSRFSRQEVATLDENGQQQWPDTSREKGGWRYGRELYGSRGRLATPDEITKHIKLAESLFPSTNRFPLPAENSDAARFIPAMAEHLVARLDRRLGRINFLFNLRWRLEGTMEQDPTTRQFTRPRDENVDTPKHRRMVVEAIAPAKLRPDSDDAEEIRWNDELRSKLTNDWEDTKGLLQKPKRRKQRQEHDEEWDEKITRISKEKEWKWEQLADAVHTQLVLLLDELGVAGETGLVAEVARFVWPLRDKRWHWRKHTIHANGEVCPSLLSTEPLPNDSEKRKIIGQGGLNMQRVETLQRFRRCCQSLAKHEERLTFGRIPGQKGLEPYIVPRGDSVHDPCPSLLEKINELRDQRVDQTAALILAEALGLELMNPADVSIEGLRKSQLKSERDVHGRYQRRKLKLDGKEADAPQCSLIVVEDLSRYLTSLDRSRFENRRLMEWSHRQVIKKLKDMAQVFGIEIMAVDARYSSRFCSKTHVPGVRVAEVKRDFEKEHPWRKWHDEKFNGKLTKRAERVRKIAKEFEKNPNATLLIEMEGGPLFLDCNGEEPRNADINASKNVGFRGVAHPDLWEFFPVVRTETATDGKLRVINRRGTLAGLNPDDAKREFAPTVPKKETKTKEGDSTAANDDDESETSERPYLFVSGAPNGERLPLADGEEVFEVSAIADGKTGYRTARGGLFWRRVADECQRRIDEINKKRLNKLIPG

SEQ ID NO: 54 - protein, 1422 aa, synthetic construct
MNRIYQGRVTKVEKLKVGKNGKSADDWEELKDWQVVLWQHHELFQDAVNYYTLALATMAKGLSPDTVESRAALAWRDEVCKSWNQATRKAIKFEGPHQRISKLLGLDPAKSDFDKCGAKLLRHTPSSAQQRAKALLQLLEEADKGDLNQICVARIRWFCTRHGALDATPKSVSHKQEIEMLKSIREVHESTSGNLAKVAAVLNVGYFLAQPPKNDIVGEVAIEEAEHQFEAALKKVPSFKKLENKFKSSIREQGKKLTVPNLGRKPKGAYSFALVFKLFPTPETWKALKDATAGSYKRSLKQTAFTSEITKDFIAEARTRQDEPIFDYLTNLLLTREADDSNHAVWFDFDLAAFIEAIKSPHRYFQDTLRREEQARKVREKLQAIDSEGKWLDVSIDKGKLKNKVKVNDSAEDEEDEAGGFTFKDDKRIKLIRQLVTDEKEGLAYLAEAENPEMEAQKAEYTIQERTLRGWPKIREGWRKLTDNKDWPKLSAEEQEKKLWAVAVAEQGEHRDDFGSSTLYKILTQAKYHPVWRDNGTQDDHADDPLRAWLQFTERRMELKDKERPIRFTPAHGVHSPRYFIFPKKYQNKSGKTSKRQVATRSFNSEHEKGVLVFAAGIIFEDNGKRQPVRSRIFYSAPRLRRDQLRSDGETDLTKARWLQPMMQAMGLGETEVTDFAKCRVTLQPESLRNIQLTFPVEVPVDKLKKSVGKESLWNRQFNLHPEGSNFYNATLRWPHEKQPVKPPKPWYEQMSAFTCLSVDLGQRDAGAYALLEARANHDFSKKPSRFIGETPRKRWRAVLAASGLLRLSGEDHKEWRANSSLDPDKKTRFAFREELFGERGRSATDAETKQCRELLAEFGVKKEDLMPADWENTLFFPEQNDKLLVAVRRAQSYVARLHRWCWFLSDDRKEKNIEKALEEIGEVKDERSASIRQLAKEKKQKELNKALRKELSGWLGRLPGLLEQVAQRCLPLRGRSWRWGQNPVKADCFILDQGGPEKPNASMKMPNGKEMNVTWIRGQRGLSFERIEQIEELRKRFQSLNQTLRRKIGSLAPKRWDDSIPDPCPDLLEKLDHLKEQRVNQTAHMILAEALGVRLTRPPANKDELRRTRDQHGAYERFRDPVDFIVIEDLSRYRASQGRAPRENSRLMKWSHRAVRNKLKELCEPFGIPVLETSAAYSSRFCSRSGVAGLRAVEVHPDMKSDFPWAWHLRRLQLYLENPQTHPLSEVALSESQHVEKLFQQLGKINKNINTERPKWRTLLAPMTVGPIFVPICDRIANPDHKKLQPVLVQSDINAAISIGLRAIADPRLWNIYPRLRTERVEKGDGLIAREKRKYGEKVKKPLNISEQAKPSKGDSRQPNFFFDVADCAKWGHATIEDPLDGKSVKLVSGKALWSTVKEQQWSRVEEINRRRIAAWESKQK

SEQ ID NO: 55 - protein, 1428 aa, synthetic construct
MNRIYQGKVTAVEIPDGNDEHGKPKWKKLDAGESALWQHHELFQDAVNYYTLALAAMAAGVDGDSEQAHALRGWAAKVKETWSNANRKAETFAGPQARLALILKLEQDKSDFEAASSRVLQSSRATLAQRSAALMQLLSVKGDLNVICGERLPWLASAHGKCGATSKAASSSQEAKRQRTIRQFHQCSEKEALAQAATLDLDLFLTQPPKDFVKGADAAKILRDYFTKASQKSELKAMASMFEKFITSQTDTLKVPSLGRRPSGLYPLAAVFRFFPTPETLAAFRIATKSLAEGKDKELVSDAVAESRVADQPHFNYFTNLALVSGNGDDERDTRAVWFEFDLAAFIEAIKAPRRYFADTIKRESAADRFRQQLAAMDGKGREASSAEEESEPLRGFEGDERIRLLKIIVQDKLKWIAEAEGDASVVGTKEYTIRERTVRGFSEIKRHWRSAVESGNGTEKRLLEILATEQTDHRDDFGSAPLYRELAKPEFHPIWRDDGTQPWHAEDPLTAWLQYKELQSDLADKERAIRFTPAHPKYSPRFFIFPKKSEAQPKGTKNRSPKAGLLSHHDPGQLSFTAGVILRTEHGLGPTVVRIYYAAPRICRDQLRSSGDVNLYEAPWLQPMMVALGLDKSPDRVNFANCRITLQATDEADIQLTFPIEVNIEKIKAGVSREAVWKNQFNLYPDGENFYNSSLRWPHEKQRAKPPVPWHEQLNSFRCIATDLGQRDAGAFARLSVLGGSDFGKQPSRFIGEAGGKKWRAALERSGLFRLPGEDAQVWREKSGKDDENPEDSGKPFDFREELWGESGRPARGWESDDTAELMRQLEVPPEDKERSLLSEGWRELLSFPEQNGKLLVAMRRYQSRIARLHRWCWFLKGDDKQKQTACDEIAECEDTRLISAEQIAQAKKHDPRLQTELESQLKKRLELAPGLLVRIANRVLPLRGRSWHWERHPASKAENTLHHLTQNGPKLGTKERPVWLRGQRGLSLERIEQVEELRKRFQSLNQTLRREVGGKPPIRRDESVPDPCHDLLDKLDNLKEQRVNQTAHMILAEALGLRLASPPADKKALRQQRDQHGTYEKIHDKAGNWIGPVDFIVIEDLKAYRTSQGRGRRENWRLMQWCIGHMRDKLKLLCEVFGLAVLETPPADSSRFCSRSGVPGFRAEEVTSGFTQREWAWLAGKKDEQGNPKPEAQRLLDLDLELSKAQAELENNWKEKKRPQPCPKRTLLVPTRGGPVFVPVVDKVEGADIPPAVVQADINAAINLALRAIADPRLWAIHPRLRSQREGGDKSGKAKKQKTKQEVSGTPTENRLLTREKRKFGEAGKPLTIKRASDAKPDDTRQPNFFADLAGLKELAEQVVKKNQREYSWLTKEWTSAEISGETSTPPLVQSKSFWGTVKAKQWERIQAINTARLAAWHDKLNPMPD

SEQ ID NO: 56 - protein, 1434 aa, synthetic construct
MPLTRIYRGRIIKIEAAKPEQGYPASFTQLDHLENALWQHHHLFQHAVNYHIVNIAAHADPEAPNDDVLRQFRDRVCAEWNGGTRKRGSGIGFGPSVAPFLGLHPQSTAEVVFAKILEGNTASPAVRHLAGLLLLEKCQGDAGISQGGRTYLPRFCAADSTFTPDFSATSQAAQAGKTELAARIHDPNTTDAELSDLAGRMSLSWTVKIQPSSCFTGAAARNRLEQAIKFFQERIAQKSDSRITAWAEAHQGATWLGELPARVAAIPNTLTIPANRKAAQELTFSTLLFQHFPSTTSASLLALFVKAPKKPNATASKKANASAKPAVDFARFGDDPIVLARGARGYVYRGFTALPVLFPDADSDPAVPARMAWKEFDIAAFKEALKAVNQFRLKTEERTARRKVLAAKLDYQLASTEQAPRDDVEATADREPDSTPRLGDDPRYLALQKLLAELAAKRVDDSEKHGIHRRGLRGWSDLLKVFRQLHEKGKRDASAYESEVISWQKEHVREAGDIQLFLKLAAPSNHSIWQPPALNEADPHPRADDVVWAACRFFEDQEELESLQEPVRLTPADAVVSPRQFIFSDITGKEGLKSVSADTWELTALARTEPGAPLGLHRLRVTVSAPRLRRDELQAIGEAATQSAWLPPILTGLLGRDEARRHAPLPVRDFAFGLMPEPRLAASPDRQPVGLCRLLVNFPVTLDVEKLANRISRPGFWTKQFNRMSERGQDLFLHLLWPGMKNAPDDDIAWWSRPEGLSALAIDLGQRTAAAAVVLEMRADDRFSGGRRPLPYPVGQTGSLAWRVAVLSARHLRLPGEDRALLPHEIDQRNPSRHEKSGAAGRVASRAEWEEALELARLLGEEKPEQRLRALPDEAASSAPPGKAPSALTFPEHNDELLHLARAATNRLRRLHSLSCRLDSDDPNIVNEASSELKADPALHRPGESAKDAAARLGAEFLSVRLQLREILLRLTARIVPRRDHEWTWREVDGGKHGELVAKQAKQKQNIPIAGQRGLSLARIEQIENLRRRFLSLNRLLEFEDRKKPDFGAKTRGIVLGEPCPDLLEKIERMKEERINQTAHLILAEALGVRLRAHDPSAAARAERRQRDIHGEYEPIPGRRPVDMIVLENLDRYLSSQDRAPSENRRLMQWAHRALVDKIRLLAEPFGIPVVTVPAAYSSRFCARTGIVGFRAEEIHTGQLDSFEWCHLEAREKADKLREHDRDLIRLRDILRKLPPRVTKQGDTLPFTLLCPKKGAQLFVPICSPDHGTTGISQADINAAINIGLRALASPQCLHARPRWRLDLSRSTDGTVRATPVGAAKEGGNKLERALWSEAQLYLCLGTDAPKAYSGKRTLLFHDSAGIAKFDRGTVRVGSTCARVATGRALFKAVKDLQWSRIHALNRARLAKAGLLTPEITHLFPRNLDESADLPDEF

SEQ ID NO: 57 - protein, 1452 aa, synthetic construct
MNRIYQGRVSSVEIPKVRTKNEWEQLPNGQDALWEHHRLFQDAVNYYIVALASMGVAPESKLTRLRGLLEQVWEVADKKGQRRQGMRESLQRAWQLVEPPTLAEAVERFQQPLRHNGVERLEQELAGESLAHDLGGEGSIQQGGMEYWPYFCQSGFKRGVTFPREASQLAKVKAVQQVARLVWHPLAGRHVHRIHSALKQFHFCNVAGGGVLLDEARTKVVFADALETLAKAHLISETQKHQLSEKLKIRIPNITEYAGGSINKDALKHRFYGFLVFKYLSPDEEGLSVLRRIYDEPSEKKSGKKKELSHKEKEEARFVSLEDDPIKMVRARAGIVFRAFTALPLWQNVESTKDLHEQSTYRHELTAGLLYQVVWKEFDVAAFKEALKVYNQFQQNVENREEKLTGFATKLLVMDGARAMQDYAGDTEGDQKLRARLENVWKECEGKPKAPANDNGEAVALPRFTGDARIDRLRQIINDDLAEEYRLTEGRRTAYGLRRRTMKGWSEVKRRWQKIVEKGNTYSKDTQAKLKSELDDLRKGEKREQIGSHKLFEALIADETAWCIWREPDDVLQKQIEKQEWAADPLETFREYCEIREAMEEVSARPLNFTPADPRYSRRLFNLKEGAGSFGKDTGEYRHDAKELAVTVPVAIRDPEGRFMIQRCRLVYSAPRLLRDRIRAEDGSYAQDWTQPMMRALFGDTEHAANPQELKDAAVQLMPDYDGKGQRRILLNFPLSLEDERIKARLGKDGLWQKQFVTWKKGAQLPFMRWPEEFDGKEPHRWWEKVSSLRVLAADLGTRHAASVALIECNKSSGGTARLIGKAGDTDWFGRYRSGSILRLPGEDAKVVRLESPLDKDARGKAFREELYGASGRPADDEECEKTMTTLAALNQSALLDGITDTAILKQRFSFPELNDKLLVALRRTQGWIATCISWQWKLTQPDSEEQRQNALGQLREQDRIPEWKQFADGKDDDIKKLREGLHGEITIQRQRVQEYLLQLTRRILPLRGRTWEWVVNPEKPDCHLLRQTDEGTGPENAKLRGQRGLNMARIEQLSELRRRWQSLNQSLRRKIGDKPLTASEMRNDPIPDPCPDILRKLENIREQRVNQTAHLILVEALGLKLRVPQMSAQSRQITDTHGEYAVGPPPVDFIVLEDLSRYLSDQGRAKSENTRLMKWCHRAIMLKVKMLAEPFGIPVLETPAAYSSRFCSLTGMAGFRAAEVGWADRHEFRWRVLTVEAAKARAEGKEPQPNAAHAAALFADLEKISHSDHPHRTLLAPQPGGPMFVTAKQIAHPAPSANRKHKEDRSVLPMQADLNAAAALALRAVAHPGCAHIHQRLRTERKKGGKNQPDSFLTREPRRFGKDKVAIVLREGDTLPKERNPNLFYDEHLVADFGRARLESDSDSTFPYSSGLGLWKGVNDRVAQWDRCKMINSARLRRWKEGKPEKDVPYQ

SEQ ID NO: 58 - protein, 1469 aa, synthetic construct
MPQQDKPPVTQRAYTLRLRGSDPSDTSWRKALWQTHEGVNKGAKAFGDWLLTLRGGLDHTLADTKVKGGKGKPDRDPTDEERKARRILLALSWLSVESKIGAPAVYIIASGKEAAEDRNGKVVAVLEEILRSRNVAENEIGEWKNDCSASLSAAIRDDAVWVNRSKAFDEAVKSVGCSLTREEAWDMLERFFGSRDAYLAPAKGSEDESSETEQEDKAKDLVQKAGQWLSSRFGTGKGADFCRMADVYGKIAAWADKAQAGMTGNDAINNLAAALNESTLASNDLQGVLGLISGPGYKSATRNLLNQLATKTTVTQQDLASLKDKATKDAQKCNQNTGSKGRRPYSDAILKNVEAVCGFTYLQDGGSARHSEFAVMLDHAARRVSLAHTWIKRAEAERRKFEEDATKINNVPSAAKNWLDEFCRKRSETSGALEPYRIRRRAVDGWKEVVAAWSKSSCSTSADRIAAARALQDDPEIDKFGDIQLFETLAEDDAVCVWHKDGDAAKAPDPQPLIDYALAAEAEFKKRHFKVPMYRHPDALLHPIFCDFGCSRWSIKFSLHEAKKNAEKKRKKKDDVQQPDSPRSLWMTLWTGKEMKLVPLRWQSKRLARDLALGQDARKDGASEVTRGDRLGRAASNVTKSDDVKISGLFEQADWNGRLQAPRQQLEAIARRVNNHGWDETAKKMRDRLQWLVTFSARLQPQGPWCEFAEKLQLRINPQYWPHADTNKDRKGHARLILSRLPGLRVLSVDLGHRYAAACAVWEAVNAEQVRGVCQIAGHEEPKASDLYLHLKRKTTKQKKGKEVEVEKTTIYRRIGADTLPDGTPHPAPWARLDRQFLIKLHGEDEGTREASNEEIWAVHQMETVLGRSVPLIDRLIAAGWGCTEKQKARLEELNKLGWTSVPKAESSGEQEENEEADVYKPSLAVDELMSSAVRTMRLALKRHGDLARIAHYLITDEKTKPGGTKEKLDENGRVDLLQDALVMWHDLFSSRGWRDEAARQLWDEHVAKLSGYKAPEEIGEESSGPERKKKQKENREKLYDVAKAMAQDVILRETLHDAWKKRWKDDDERWKKQLRWFKDWVLPRGKHASDPTIRKVGGLSLPRLATLTEFRRKVQVGFFTRLQPDGTRAETKEQFGQSALDALEHLREQRVKQLASRIAEAALGIGRMRRPMGGKDPKRPDVRVDEPCQAIVIEDLTHYRPEETRTRRENRQLMTWSSSKVKKYLAEACQLHGLHLREVSASYTSRQDSRTGAPGIRCQDVPVDEFMRSLFWRKQVKQAEAKQSENKGDASERFLCDLNAKWKDKTVADWEKAGVIRVPLRGGEVFVSADPNSPAAKGLQADLNAAANIGLRALTDPDWPGKWWYVPCDPASFKPTPDKVKGSAAINLDMPLKDASVVSGDATMKKAGKAKSQSKEVVNLWRDVSSREVSTTDVAWEVYSAYQNQVQTRVVNILRRSACPKDNREKQS

SEQ ID NO: 59 - protein, 1473 aa, synthetic construct
MPQQAKPPVTQRAYTLRLRGAGPSDISWRKALWQTHEAVNKGAKAFGDWLLTLRGGLDHTLADAKVKGEKGNPDRDPTDEKRKARRILLALSWLSVESKLGAPAGFIIASGTEAAEDRNGKVVAALEEILKSRNLAENEIDEWKNDCSASLTAAIRDDAVWVNRSRAFDDAVESVGCSLTREEAWDMLERFFGSRDAYLAPAKVTEDESSEEEQEEKAKDLVQKAGQWLSSRFGTGKGADFSRMAAVYGKIAAWADNAQAGTTGNQAINKLAADLSEFTPASNDLQGVLGLISGPGYKSATRNLLNGLATKTTVTQQDFGSLRDKAADDAQKCKQNTGSKGQRPYSDAILKDVEAVCGFTYLQQDGGAARHSEFAVILDHAARRVSLAHTWIKRAEAERRRFEEDTQRLANVPGPVKAWLDSFCSERSATSGALEPYRIRRRAVDGWKEVVTAWSKSSCSTSADRIAAARALQDDPEIDKFGDIQLFEALAEDDAVCVWHKDGDAAKAPDPQSLIDYALAAEAEFKKRHFKVPAYRHPDALLHPIFCDFGKSRWDICFDVHKNVQTPFPRALCLTLWTGSGMVSVPLCWQSKRLARDLAIGSNATNAGYVEVARADRLGRAASNVTKSDEVKISGLFEQADWNGRLQAPRQQLEAIAVVRDNLSLSAQERERRMSSMKDRIRWLVTFSAKLQPQGPWCEFAKTNQLRIDPQYWPHADSNKTRKGQARLILSRLPGLRVLSVDLGHRYAAACAVWEAVNAEQVKKACQVAGHEAPKASDLYLHLKRKATKRKKGNQVVVEETTIYRRIGADTLPDGTQHPAPWARLDRQFLIKLQGEEEGVREASNEEVLKVYHLEAELGRTAPIIDRLVKAGWGQSGKQRTRLEALRELGWKPASEAVVSDGMGADEGEVRKPSLSVDELMSSAVRTMRLALKRHGDRARIAHYLITDEKTRPGGVKEKLNENGRIDLLQDALLMWHDLFSSRGWRDDVARQLWDNHVAKLSGYKAPEEIGEESSGPERKKRQKENREKLHDVAKALAQDVTLREALHDAWKKRWKDDDERWKKQLRWFKDWVLPRGKHASDPAIRKVGGLSLPRLATLTEFRRKVQVGFFTRLQPDGTRAETKEQFGQSALDALEHLREQRVKQLASRIAEAALGIGRMRRPMGGKDPKRPNGRVDEPCHAIVIEDLTHYRPEETRTRRENRQLMTWSSSKVKKYLAEACQLHGLHLREVSASYTSRQDSRTGAPGIRCQDVPVKEFMRLPFWRKQIAQTEKKQAEGKGDARERFLCDLNAKWKGKSEADWEKAGVIRVPVRGGEIFVSASRDSQGAKSLQADLNAAANIGLRALTDPDWPGKWWYVPCDPASFRPIKDKVEGSAAVKPDQALRQSAQAQSGDAAKDKKKRGNKSAGKSKEVINLWRNISSSPLECIEFGEWKEYAAYQNEVQCRVIRILEEQIKGRDKQLHEGAKEDDISF

SEQ ID NO: 60 - protein, 1518 aa, synthetic construct
MDMKENIGQTQRAYTLRLRGGDPKDQAWRDALWHTHAAVNRGAKVFGDWLLTLRGGLDHKLADKDRKSEKSNQDDTHRPQKNRRILLALSWLSVESKLGAPQKYIVAEGHEETEMRKNNVIAALENILKKRGLPEGDIDEWKRDCGPSLSANIRSDAVWVNRSLAFDDAVQNVGNSLTRDEVWDMLEGFLGNSSTYLAPKKSSDEEVSDEDSDSKAKDLVQKAGQWLSSRFGTGKGADFKRLAEVYQKIAEWAETTTPNMTGSAAIESLAATLREFSPPSNDLQSVLKLISGPGYKSATRNLLNQINRKPSIDQEDLTKLKKTAEENRKESESKIGSKGRRPYADAILREVESECGFTYLQDEGPARHVEFAVMLDHAARRVSLAHTWVKLAEAERCKLENDAIKIEQIPEKVKAWLDRFCEERSDESGALEVYRIRPRAVDGWREVVDVWSKPDCKTEEDRIAAARMLQDNPNIQKFGDIQLFEALAAEDALCIWHRDGDLEKEPDHKLLLDYVDARDAEFKKQQFKVPAYRHPDPLLHPVFCDFGESRWKIDFSVHRAVSDLENTKKKVAQIEKQVEKEKKSLQKVSESARTAAQERLSLLEQELKKAKEELDCLTAKNRMTMTLWNGKMMQQTHLCWQSKRLAKDIGLKKNGEGQDSMEVTRADRLGRAAAKAAPHAPVKIAGLFEQNEWNGRLQAPRTQLNHIARYVEKNGWDQKAEKMRNAIKWTVTFSARLRPMGPWCEFARNAGLIPDPKSHPHSGMNKERKGLARLILCRLPGLRILSVDLGHRYAAACAVWESMSALSLKHDCRAVGCDGPTENDLFLHLKKRVKKQRKNSEVEVTETTIYRRIGPDMLPDGKPHPAPWAKLERQFIIRLQGEEEGVREASNEEIWAVHQMETLLGKPNPLIDRLVASGWGNTKKQQARLDALRDLGWKPVEKAAIQPDALAEEEEGTFRPSLAVDELMFAAVRTMRLALRRHGDRARIAHNLITDFQTMPGGRKCALNENERIELLKDTLQDWYGLFSSRVWKDSEAEQLWQNYIRDLAEYKAPENLVQNTTGMDHGKKKKLDREKFRDLACLLARDPDLRKKLHDAWKTRWEKDDVRWRELLRWFRNWIRPRGNFGDNPAIRGVGGLSLTRIATITEFRRKVQVGYFTRLRPDGSRNEISESFGQGMLDALEHLREQRVKQLASRVIEAALGVGRMKRMQKSRDPRRPVAPVDKPCHAVVIEDLTHYRPEETRTRRENRQLMNWASARVRKYLQEGCLLYGLLLREVNAAYTSRQDSRTGAPGIRCQDVPVVEFMCSPFWRKLVDRTAKKLPADEENADMRFLSAVKKRWDGRTLSELRKAGVVRIPLKGGEVFISADPQSPAAHGIQADLNAAANIGLRALMDPDWPGKWWYVPCDPKTCKPAKENVQGSAVVNVDQPLCLSAVITELNRNGQKSKGKSEGKTKNIINLWRDISSSSLDSAEAGAWQDYKNYWQHVRVRVLHILEEQLKRRDPLWPGHSEEEDLPF

SEQ ID NO: 61 - protein, 1536 aa, synthetic construct
MPDQTNPPLTQRAYTLRLKGAQKDDTSWRCALWQTHEAVNKGARVFGDWLLTLRGGLDHTLADASLKSGQEKPDSTLTDEERKARRILLALSWLSVESKTGAPENFIVASGQDSEKNRADCVISALKEILESRKVDKNELDAWINDCSASLSSAIRDDAVWVNRSKAFDEAVKSIGPSLTREEVWDILGCFFGSREAYLAPGQSSDEDESSEAEQEDKAKDLVQKAGQWLSSRFGTGKGANFSHMSKVYEKISGWADSAQAEMTSKEAINDLAAALSEFAPASNDSAGVLALIKGPGYKSATRNLLSLIALQTTVTQEDLTKLKELAAKDKVECATKIGAKGHRAYSDKILEDVESACGFTYLQQSEEESARHSEFAVMLDHAARRVSLAHTWIKRAEAERKKFEKDSKKINCLPEAVRTWFDNFCIDRSELSGALEPYRIRRRAIDGWEKVVAEWSKPSCGNCADRITAARMLQDDPDIGKFGDIQLFEALAEDDALCVWHKNGNINEASDPQLLIDYAAATEADFKKRKFKVPAYRHPDALLHPVFCDFGNSRWDIDFSIKKAFNDLDKQKALLNKKEEEIIRAEQKLNIKNSKDVESQKKLQQEKEKLKKIQQKIAWLEEKNGVEMALLNNGKIGKMALRWQSKRLTRDLALDQSDNDCGVSHDVSRADRLGRAASNATGGKVNILEIFNQDYWNGRLQAPRQQLQDIAAVRDNPSLQAAERERRMSIMKDRIQWIVTFSAKLRPKGPWCEFAELNQLNIDPKYYPHAKENRGRKGMAKLILCRLPGLRILSVDLGHRYAAACAVWEAVSADDVKLSCACAGHEEPNERELYLHLKEKKSVEQKNGGKTETEQTIIYRRIASDTLPDGTPHPAPWARLERQFLIRLQGEDEIVREASNEEIFNVHRAEAELGRTVPLIDRLDKAGWGKSRKQKERLDGLRKLGWERVSDGTAQDVPDTGEDYWAKPSLSVDELMSSALRTLCIALKRHSDRARIASAMTAEYKTMPGGRKYYFTGPRELSASDDKSTIDNKHIEFIQDALVLWDGLMTSRGWRDDAAKQLWNNHIAKLSGYKAPEEISDESSKTERKNKQKENREKLRGAAAALVSDTNLREALHAAWKKIWEDDDELWKKRLRWFKDWILPRGKAAKDPAIRKVGGLSLARIATLTEFRRKVQVGFFTRLHPDGTKTEITEQFGQSALDALDRLREQRVKQLASRIAEAALGVGRVPRTKGGKTPKRPSARVDEPCHAIVIEDLSNYRPEETRTRRENRQLMSWSSSKVKKYLSEACQLYGLHLREVSAGYTSRQDSRTGAPGIRCQDVPLTEFILSPFWRKQVAQAEGKGDARDVLIRDLNHMLEDKSASDGEKPKVLRIPLKGGELFVSADKKSPASKGLQADLNAAANIGLRALTDPDWPGRWWYIPCEPGSFRPVKDRVEGSAAVDPNRALRQDAQPQGSDAAADKKKRAKKSTGKSQEKEIINLWRDISSSRPDDTAAGKWQTYDEYQNRVQYRVVDILREQANERKKRTSSRCDKP

SEQ ID NO: 62 - protein, 232 aa, synthetic construct
MKLQNGIIFYIPAWNTSKIDPTTGFVNMLKINYTSVEAAVNFFKKFDSICYDSQKDIFAFSFAYTNFGVNVDYKNKWTVYTYGDRIETYHTNNNQYDYKTVFPTELLKSVLKSVGIAYADGHNLVDDICAVKFKDFYVKVLNVLKLTLQMRNNNSKTGEDYIISPVLNSMGQFFDSRNYSGSNASLSVDADANGALHIAKKCLWAINQIKIGNDKISISNREWLEYMQRGNQ

SEQ ID NO: 63 - protein, 173 aa, synthetic construct
MDMGLNNLFAVVTTNGYIVLVKGGVIKSEYYWWKRGIATYQAVRDLLRNAGFPTWLTYHERYIEAMYVRDEKLRHLYRTSIRFLAETLWNRGVRKLYIGYPIMLSQDNGNEYNTNIWWYRKIVLWIVDVFREYDIEVELVPEYYTSRECSICGETRMGGYIEDYTYAIRLGGR

SEQ ID NO: 64 - protein, 120 aa, synthetic construct
MFIATTKRLGYTMAREYLRGLKESLRKLSKNHRTRLPLMGYFRIERWIDWQSLKHGVPRVVVNARNTSSECPKCDYIGLEEISYRRLRRPRCGFEGGRDEVGKLNVRKKAPRILGINGEL

SEQ ID NO: 65 - protein, 117 aa, synthetic construct
MAILLCGNSSFSCRSTFLHLPTPTPFHPLLSGSQLCQSKALKYARKVVIINRWEPTSQVCSSCGYRWGKLNLKIRTVVCLGCGETHCRDWNASKNIEQVGVGHTHDSKQTGLRCTRK

SEQ ID NO: 66 - protein, 131 aa, synthetic construct
MGCGLVIIEDYGGIEPDEDPHKRRLLDKFPLYEMKQAIAHRLERDGLTLREADSHYISSVCPRCENKDTRQHNTRTGMFHCGACLFERPADWVASYWLLFHGGADMTVWKYKLRREKELSDALNSRKEALA

SEQ ID NO: 67 - protein, 165 aa, synthetic construct
MLMVRWFALVPIEVGVDPPKSNSKGYVKPIYNDEGKKKKQRITNLKSIGQRDPIGDGEAFIDMGLNNLFAVVVSDGSALLVKGGSIESEYYWWKREIATYQSIRDLLRRLGVSTWIYYHEKYLDAIYKRDERLRHLYITAIRFLADDLYRRGVRKLYIEYPIMFS

SEQ ID NO: 68 - protein, 123 aa, synthetic construct
MRGQDTNALTCYDYKGESHIISGRQWLSVERYFHKKIAHYQAIFDKQQSAKGVKHPKKSKRVLQLFKKRRKQTFHILHCMTKKAVDIAVRQGCETIVIGDIAGIHKKLNLGKKNESETAYFSF

SEQ ID NO: 69 - protein, 173 aa, synthetic construct
MRTSSKREQAIRVSSQSIVAVSPTERAIRETASALKNPRPLKSLQCKLRREQRRLSRKMPKSRNRENARIYVARLYERIVNIRKDFLHKLSTRLARENQTVAVEQQSLSARGDCYAESQALCKKTQYVNRRSPKDFSVSCKQDEVSVHPVREHDKSSSLQPVHPEAGSGRASL

SEQ ID NO: 70 - protein, 169 aa, synthetic construct
MTNAQRKLSRRIVRANREHRPLSQAKNLQKQRKIVAKLAAAIMRRRKNFLHKTSTALIKNHDLVAAEELRSKNMLKNHALAMRLQDSGWRTFLGMLTYKAKLYDKIFIAVNPRNTTQTCHHCGYVLTGPQKLTLKERTWTCPICKMHHNCDVNAAKNILTKAKQQLTAR

SEQ ID NO: 71 - protein, 391 aa, synthetic construct
MATASDQQSLTAFAAGTTTETVTQTVVLPLETSERKNSKVETAIDEFQAMCGYLCDMMPSVGPHAWSSNNSTLYRLVTREFPEDERTVSGKMCDTAKRHVVAAFQSYREKNRSGDRPSLGGGNYFQVSAQRLTITKNDRGYGVKANFIPYKPEWFHIDPRPYAQEYLERVVDGDASYGTGEFRLSENGDLALHLPVSWEVEVYEPADVSTTIGVDIGENVIYAAAVVSSDGVNAVEMEPGREFRHYRERLQEKRDRLGEQGDLRGLKQTRDEQRRYTEQVLDTASREIVDLAVEHAPAKLRLEDLTHYRETAADPIHDWPFALLQEKIAYKAKAEGIPVETVNPRNTSVTCRKCGQTNREYRDGDEFSCTRCGYEVHADVNAAINIAQESS

SEQ ID NO: 72 - protein, 389 aa, synthetic construct
MATSSQQTLGEFTAGTGTQTVTQTVVAPLETSNTKTEKIATAVEEFSAMCSHLGDMMASIEPHERIPNNPTLYRLVTREFPSDERTVGSKVALAASRHVAAAYQSYHARDDGGVRPTFGDETFFQLDNQSFEIVENDCGFGVKARFIPYHPVWFHVNPRPYVQDHLDRVCNGDASVGTAQLRLHDDGGVSLHVPVSWEVDVYEPGELSRTVGVDIGENVIYAAAVVDGSGDVEAVEMQRGAEFRSYRERLQRKRDRLQKQDDLRGVKATRDDQRRYTEQVLDTASREIVDLAVDHAPAVIQLEDLTHYRRDARDPIHDWPFALFQEKIAYKARADRIPVEVIDPRDTSITCRKCGQTNREYRSGTDFHCTRCGYEVHADVNAAINIAND

SEQ ID NO: 73 - protein, 318 aa, synthetic construct
MDAIKNLKEGYVSAAVHEICRIAVEEKALIVIENLNYGFKNSRKKVEKQTYQTFERMLINKLQNLVFKDSEPGSPGSVLAPYQLCGPIDSMEDIPSHCGMLIHVTAAFTSRIDPVTGFVNVFNLSDITNNTQRADFLSRMTSIRYNSDEDVFEFSFDYGNFRTRLTPKVRKWTVCTYGDRYTYNRKDRTLVRISPTSMIKDALTKARISYEDGSDIMMDISESDAVRQVFEAFRAILDMRVTTREEDNIVSPVRDSFGRHFVSGTSGPELPQDADANGAYCIALKGLLLAKKIRTSDGDRLPLLNNNDWVEFMQTRVM

SEQ ID NO: 74 - protein, 381 aa, synthetic construct
MGSETVQIARTIVCPLESSQRKREYVREAVGEYQGCLETMAAMMPSITPHDWEAQNTTVARVAKEECNSERLYAHDRNDAAYKVAEAFGSRVSNNHTGEYPSFGDGQYIRICSCCSRVELVENDRGYGLWVKLLKRSDGVWFHLRGGGYQYDQLKAIVDGEASLGNVELHLTGDGVTAHISYIEDISTVEMDSCSSVLGVDCGQRVLYASAVRADDGDVVDVHVESGDEQRHYRERLSNTQSRRQQSGAKSRAVGVSERQRYTEQVMHTASRQIVDQATEQMPCCIVLEDLTGYRDTAQAPNHEWPYGSLQSKIAYKATGAGVPVRSVDPAYTSQRCRKCGTTDRASRQGIDFVCTSCGYDVHADVNAAMNVADRGYADIA

SEQ ID NO: 75 - protein, 387 aa, synthetic construct
MIKVVRLKLLPSPEQAGVLAETLRVCNEQAVEVARVAQERRVFGGYDLRAITYGQVRSAGLGAQAAQQVIRKVVDAYKTRRANFRSGRYGPASEPRAQRIMGTAITFRPNAAQPYDDRILSWDHAHQTVSIWTLAGRQRIPFTGHRDHLALVAAHRRGETDLLCDRRGRWFLIATIDQPDPQPAEPEGWIGVDMGIVQIASTATDNATPGTNWSGGAITRRRKKNLRLRRRLQAKGTKSAKRLLKKRSGKEARFATDTNHQVSKKIVAEAKRTGRGIAVDNLTGIRERARHRKPQRATFHSWAFAQLGGFLTYKARAAGVVLVEVDPRHTSQQCHHCGHTSRSNRTSQERFTCTQCGVSLNADTNAAINIARRARDGWGAVNRPHAA

SEQ ID NO: 76 - protein, 372 aa, synthetic construct
MKNPKTIKRVVSTVIPKNDPSLPFLLREYDAYRKALMSRVKSVVRPVIKDPSNHVHAKQKWMIDTKLSRFLSNLPFHPMPFHRQDIWLEQREQRFFMHFRTGQGELACRIKIAPKFCGQIAEACGPNNPMLRQTELVEDPTRRKIRVHIVLAIPAKKPYKPDGWVGVDIGWNHLATTAFVDSKTKKASHVTSHSKDWKERILNLKNLLRQHQRTEKATKVWRHRLANVTRNAVGETANEIAKKALQLHAGVKIEKLTFRSHTKRWLVPRYKLAMAIKNVCEREGIPVKEVVAAYTSQICSRCKNRKSLRQARKEAAAKCGQAEHGEEEKSGAVRCRFRKLISRQRTGKRFFCRDCGYQADADVNAAINIAKS

SEQ ID NO: 77 - protein, 360 aa, synthetic construct
MLFKTLVLCRNMYNKLLQELNKQKKVNIAAVRHKVKEFRKEDPELQKIYAAALHYEAYRLKSNLRGLARLKKKGYDVGALKPRLAKNSKTFCYQQHGFELIKRPEKKFDILKLSKIGDVKVRAHQDIQGKIKMITIYKKIKHWEALISTDDDIKRTKGKKKIGIDVGIKSFLVTSEGERIDNPLFYKQQLDKLKKLQRIHSKKKKGSHNRRKAGREIARLYEKIDRQKKDFFHKVTKKLINDCSVIVMEKLDIKNMTDKKKNKDKKIPSKIRKTKNRNTLDSSWYLFKLMLKIKAETAGVEIIEVNPAYTSQTCSECGFRQKMPLDKRVFNCQKCGIIIDRDHNAAKNIYARSLACVETH

SEQ ID NO: 78 - protein, 388 aa, synthetic construct
MLFQCINVRRVAFNYALGKWQQMYDAWKEDNTKPQPSAFLIDKLFNADKANLYPWMYDAKSKLIVPSCVGQEAIKADIKSAFNNFFRRVKNGETPGYPKFKKRGMGESFTLTSVVVNNNHVSGNKLILPKGWGTATLGDSIPEGKIKSTTISYRAGKWWVSFLIEGDYEYESCGDKIVGIDMGVAKFASLSNGIVYDSAKALEQNQAKLTKLQRQLAKMEKGSNRRQKQKERIAKLHKRIADIRVSHAHQVSSELSKKYGMIALEDLKLRNMTKSAKGTAEEPGKQVKQKSGLNRSLLNQGLFEFRRQLEYKTVRHGSVLTLINPAYTSQTCSACGYVSKENRKTQAIFACVECGHIENADINAAKNILKKGRFKQYGDIGRNLRTWT

SEQ ID NO: 79 - protein, 398 aa, synthetic construct
MPDEQTSIDAFTPATDEIKVTRTIRCKLETSQRKNVLVERSINAYQEVAAEMADVLPSYPEWEWEPRHSHMYHHAKRFLPEYPDDQPGRGFKTTLALVAQQQVAEAFKSWRERGKPGDSPQGDYGDGSFLQLRADDVTIVENDHGWGLKASFISYNPVWFHIDAGVFQREFLERITDPDDAASHGSAELHLHDDGTLFCHLTVSWPVECYSADDVTTVVGVDLNDDPLFAAAVVQGDGVEAVDIESGSEYRHHRERMKQKRAEAMADENLRAVKEARLNYQKYTDHITNVASRRVVDLAEEHAPAKIHLEDLTHYRETAKDPIHDWPYALIQEQIVYKAQEVGIPVAMVDPRDTSTTCRKCGETNPAMRADRDFECWECGYEVHADVNAAINIAQRSQ

SEQ ID NO: 80 - protein, 397 aa, synthetic construct
MPEETVVQTVVLDLDTSNRKNKKIQTAIDEYQEMCSYVSDLMPSFDESQWSTMNPTFYRRLTDEFEDRTINSYTSLEAIEDVIGAWKSWRSNGKRGDRPEFGHGNYFNIKKDNIKIEENESEYGIRLNFIPYDAEWFHIEPNKYQDSYLSKIADGNAYWGTCEIHKEGDSVYAHLCVQWEVEVPQSDGVNNVVGVDIGENVIYALSVLSEESVKQVEMKNGSKFRHHRERLKQKRKEMMAKDDLRGVKKCSGEHLRYTEQVLDTASREVVDTAVEHKPCKIVLEDLTNYRRSAINAIHDWPFALFQEKIMYKSKAEGISVETIDPTYTSQTCRCCGNQSSSNRDGHEFVCSDCGYQVHADVNASMNIALEGCEYERDDYSKTNENDEKLDRSSLLEF

SEQ ID NO: 81 - protein, 380 aa, synthetic construct
MPKIFGAGFHEIDFDCSVLRIRLDDMPEGEWLDLGFIPWPRDYRPSKDEVQDKITSVHVNFVGTRARVGFRFDVPHQPSRFDCTQDELDELRSQRFPRQAQDQQFLDAARKRLIESFSGDAERNLRLLAVDLGETGACAAVYQGRSHQEDVALEIIKIDRHYAELPPILERDKAQDSPPKFSRDDPRGLRKEHVGRHLTRLAEGAANIAGHRQKDGTTPTTADDHDFRGLKRHVGWMIRDWVRHNAAQVVAAAEKHQCDLIVFESLRGFRPPGYDQLTAEKERKKRWLAMFAYGRVRHKVTEKAVERGMRVVTVPYFMSSQVCSACGREQSNQGRWRKNKKQRKFKCEYCGAEVGSDANAARVLARVFWDEIRLPQGKNN

SEQ ID NO: 82 - protein, 364 aa, synthetic construct
MQTDIQLKSYKYQLRLNKSQRVIVDRWISGTRYIYNIALQTKVWAWEGARVSLSCYDLQKQLTEVRSVFDWVNEIPRDVCNMVLERLDAAYKSFFTNIKNKVKTEVRYAGRLRWNSINFKSCKYAGGMIFQLPKLGKVRIENDLVPDGAECRTCTISRKDGKYYLSVAFKMPIEQLPKTGKSIGIDVGLAYFSADSDGNTFNLPYDLKRHEARLRILNRAIARRKKGSIRRNEALRQRRKLYDKISQQRNHFLHEITNYYVSNYDLICVENLKVKNMVKNKNLSKSILNSCWSNFRRILEYKAKWYGKKIVAVMPHFTSQMCNLCGHTEADNRKSQSVFLCLNCGHSENADINAAKNILKAAGV

SEQ ID NO: 83 - protein, 353 aa, synthetic construct
MRTIVDEFQAMCKYLSTMIPSVPEEEWNARNPTLDRMVKAEFSDKRTVSAKVRQKAVQHVVAAFTSWRERGKHQNDDRPTLGNGDFFQADNQMVELSKNDRGYGIRINAIPYETEWFHIDASPYSRDYLKRICDGDATYGTAEFRLGADDDVHCHLPVKWDIEVYEPADVTTSVGVDIGETVVYAAAVVSGDDVNQVEIKSGREFRHHRELLDQKRSRLSEQGDLRGVRQCRGERERYTEHVLDTASREIVELADIHRPAVIVLEDLTNYRETAEDPIHDWPYSMLQEKIAYKATAAGIPVETVDPRDTSITCRKCGQTNRDYREGTGFHCTRCGYTVHADVNAAINIAHRYD

SEQ ID NO: 84 - protein, 391 aa, synthetic construct
MSRTEQAPLTDFAAGQSTVDVTRTIKLPLESSQTKNEVLRDGIAAYQQVAREMCDVLPSYPEHEWTPRHGHMYHHAKRFLPDDRRYKTTLAQQAQQDVAESFASWRERGQDGQPPRGEYGDAPYLGLRQDDCEIEANDRGYGFKARFVSYNPVWWHIDAGEYQREYLARLCDDDDPARAGSAEVHLTGGRPVAHQTITWPVDVYEPGDVTTHVGVDLNDDPLVAAAVVEGDGTVAAVKMESGAEYRHHRERVKRRREQAMAEGDLNALSEARLTYQKYTDHITNVASRRVVELAREHAPVVIHLEDLTHYRRTAEDAIHDWPFAEIQEKIAYKATEEGIPVEAVDPRATSVTCRQCGAENPSARDGHEFNCLDCGYQVHADVNAAVNIASK

SEQ ID NO: 85 - protein, 375 aa, synthetic construct
MTVNIDNKIKIYYNKNKKKGCDILQLLTIKIRFFPTNPTILKKMSQEYIQVVNQLTQQAAENNSFPKITTKDVKTVLPSAICNQAIRDAKSVFNKYRKTKKLSILKNPVYYINNQNYTILDNVIAFPISINNKTKKTYFKAIPTEKDMKLIKKSKLGLMRIVEKSGKWYAQVSIQIPEQENVNSENIMGVDLGLKVPAVAVTSTGKTRFFGNGRQIKYIRRKYQSRRKKLGKLKKLHAIKKLGNKESRWMKDQNHKISRQIVNFAIQENVSVIKLESLANIRQTTRKSRKNAKNLHNWSFYQLQKFIEYKANLAGIKVVYVNPAYTSQTCPCCGTRNHANDRKYECQCGFKAHRDRVGAINIMRQPVTDGNSLSA

SEQ ID NO: 86 - protein, 386 aa, synthetic construct
MWKINLFVKRTITVKLEPSKAIGQTVRIFNEATNFFLRLGFTNRTHSKRKLQALGYYEARDKWPALQSSLVQGARDCAADMLKRERGRRLPIKKPGSSARFNQRTFKAFLDSGSLSLTTVEGRLKVPLRIPEYFRKYECGKVNALRVREVDHDLLADLVVELPDVPIKYVINPRVVGIDRGINNIAVTSDGRFFNSKHLRSVQGRYAYLKRELQSAGTRSAKRHLKRLSGRERRFQADVNHRIAKEIVGADFDVLALEDLSIKKDKRLGKEFNRKLGRWAFAQFEAFLRYKSEECGKQVISVPPEYTSQMCSRCGNLGTREGHSFRCPVCGNSLNADLNAARNIASLGNALVGRPTVNGPIVATSDAEHRASVEVSYKPLISMCGS

SEQ ID NO: 87 - protein, 187 aa, synthetic construct
MNAYFNKKRAKYMSYVGDRGTSNRIKKLTRKRNNKIKYFFHNVSRYIVDWCDENDIDTLVIGKNKNWKSESNLGKKNNQNWISIPHNMLVEQIEYKCEDIGIEVLRQEESYTSQASFLDKDEIPVYKPGEKNTYEFSGKRIKRGLYKAKDGAIIHADLNAAYNIARKKLGDIFSQKIYLHPKIVNIE

SEQ ID NO: 88 - protein, 104 aa, synthetic construct
MIARYKNKQLRHKIYQSALKGKLNAIIDKAREYGVPVLMIDPRNTSKICPIHNTSIEYGEARIGICSKGGERWHREVVALINVLFYGPMPFMRAMPRRASVFQA

SEQ ID NO: 89 - protein, 104 aa, synthetic construct
MIARYKNKQLRHRIYQSALKGKLNAIIDKAREYGMPVLMIDPRNTSKICPIHNTSIEYGEARIGICSKGGERWHREVVALINVLFYGPMPFMRAMPRRASVFQA

SEQ ID NO: 90 - protein, 132 aa, synthetic construct
MKIKLEYCPFGDIGEKEFTLASVEVETEHNDLFEFINPVVKVQSYPAASDQKNKLKAAAEKASVRKSVEEMALRAWDRELKVSFGYQGHSYSPNNLDLSHAVYNLPSFKVLNIEGNEDFQSKLPRIPKGCVS

SEQ ID NO: 91 - protein, 192 aa, synthetic construct
MIQKFKSVSYDKADGLFKITFDYKDFTDRAFGKRNEWTITSYGSRIENSRTPGSNKWKSEEINLTEEYKKFFDKMNIDFTRTDLKEQLLQQDSKDFYAGFFRLLRLTMQIRNSKIGTDIDYMVSPICNASGTYFDTRIGRAEWPLDADANGAFNIARKGLLIMDQIRQSTGYKIKTAISNQEWLSYAQEHTI

SEQ ID NO: 92 - protein, 392 aa, synthetic construct
MATSSQETLSTFTAGTGTQSVTQTVVAPLETSNTKTAKVAAAVEEFGAMCSYLGDMMASIEPHERTPNNPALYRLVTREFPSEERTVGSKVALAATRHVAATYQSYQARHDGGEGPSFGEETFFRLDNQSFDIVENDRGFGIKARFIPYDPVWFHIDPRPYVRQYFERLCDGSASVGTAEFRLHDDGGVSLHLPVSWEVDVYEPGDVSTAVGVDIGENVLYAAAVVCGDSVETVEMQSGREFRHYRERLQQKRDRLQQQDDLRGLKATRDDQRRYTEQVLDTASREVVDLAVEHAPAVIQLEDLTHYREDARDPVHDWPFAEFQEKIAYKARADRIPVEVVDPKHTSTTCRRCGQTNREYRSGSDFCCTRCGYEVHADVNAAINIAQTPGGE

SEQ ID NO: 93 - protein, 389 aa, synthetic construct
MATSSQQTLGEFTAGTGTQTVTQTVVAPLETSNTKTEKIATAVEEFSAMCSHLGDMMASIEPHERIPNNPTLYRLVTREFPSDERTVGSKVALAASRHVAAAYQSYHARDDGGVRPTFGDETFFQLDNQSFEIVENDCGFGVKARFIPYHPVWFHVNPRPYVQDHLDRVCNGDASVGTAQLRLHDDGGVSLHVPVSWEVDVYEPGELSRTVGVDIGETVIYAAAVVDGSGDVEAVEMQRGAEFRSYRERLQRKRDRLQKQDDLRGVKATRDDQRRYTEQVLDTASREIVDLAVDHAPAVIQLEDLTHYRRDARDPIHDWPFALFQEKIAYKARADRIPVEVIDPRDTSITCRKCGQTNREYRSGTDFHCTRCGYEVHADVNAAINIAND

SEQ ID NO: 94 - protein, 391 aa, synthetic construct
MATTDQQPLSAFTAGTGTETVTQTVVAPLETSDRKNAKVQTAIDEFQAMCSYLADMMASIPSHERRPNNPTLYRLITREFPGDDRTVAAKVALAASRKVAAAFQSQRARGDGGELPAFGDGQYFMLDNQAVTLVENDRGYGLKANFVPYKPEWFHINPRPYVRDYLDRVVNGDASYGTAEFRLSDDGRLAAHIPISWEVDVYEPGDVTTTLGIDIGENVLYAGAVVDSDGVATVEMESGGEFRHYREQLQRKRERLMQQDDLRGLKACREEQRRYTEQVLDTASRRLVELAREHAPVKIHLEDLTHYRETAHDPIHDWPFALLQEKIAYKAAAEGIPVTAVEPRNTSVTCRKCGQTNREYRDGSDFACARCGYEVHADVNAAINIAQKNGD

SEQ ID NO: 95 - protein, 346 aa, synthetic construct
MILKTSASGSRRTLVGLKVQSPENLYRLLTREFDDCTLSKANAHEAAKKVTEAFASWRSNGYPGNRPRFGNGDYLRLRGDQLIIEPNDRGFGAKLSLVPYHPEWFHLRIGDYQREYLQAVVDGEATTGSAEVHLRDDGTANLHLTVKTDVRVYKPGDLSRWVGVDLGENVLYAAAVVHSDGTVAAVDMESGRESRHYRDRLSRKRAELQSKGDLRGVKKCLNEHERYTDHILNVASRRIVDLAADHAPCGIRAEKLTGYRDTADDPIHDWPYAAFQEKIAYKATAAGIPVVFVDPAGTSTTCRKCGQTDPEYRSGTEFACRRCGYEVHADVNAAINIARRGSAPAD

SEQ ID NO: 96 - protein, 323 aa, synthetic construct
MIVKYKAIVVLENLNVGFKRMRGGIAERSVYQQFEKALIDKLNYLVFKDEEQSGYGGVLNAYQLTDKFESFSKMGQQTGFLFYVPAAYTSKIDPLTGFITPFSWKHVKNREDRRNFLNLFSKLYYDVNTHDFVLAYHHSNKESKYTIKGNWGIADWDILIQENKEVLGKTGTSYCVGKRIVYMDDSTTGHNRMCAYYPHTELKKLLSEYGIEYTSGQDLLKTIQELDDDKLVKGLFYIIKAALQMRNSNSETGEDYISSPIEGRPGICFDSRAEDDTLPHDADANGAFHIAMKGLLLTERIRNDDKLAISNEEWLNYIQEMRG

SEQ ID NO: 97 - protein, 346 aa, synthetic construct
MKQFTNLYPVSKTLRFELIPQGETRAHLNDLIAEDEQRAEDYKEVKKYIDRYHKWFIDDCLSKVPNSPLGEEMIRLLNKYIEANDEKDGEGPDKVQDDLRKLIVKALKSGVSLDKDPEQEERFKSLFSEELVKNILPDFVQTEEEKNNVAKFREMTTYFVGFHKNRKNMYSEEAQTTAISYRLIHENLPRFLDNIAVYEKINPVLSDEIASLEEQLHQNGYVEACCIDSLFSVDFYLRVLTQRGIEQYNAVIGKIVNDESDEIKGLNERINLYNQVHKKEKLPLFKPMYKQILSDREQLSWLPETFENDASLLDAVKGFHQDLLSNDILSRIDKLLSSFIKLVEVA

SEQ ID NO: 98 - protein, 323 aa, synthetic construct
MLLEDNKGRLFAWINLQGKDSRFAAKVVVTNMVNTRTGEIVNFTSKLGCLMPLECGQWHYERFIKQGTLQSSKLIYRKDEFFLACTFAFETEDIECVTYLGIDRGIEEIASYAVITDDLELLDQGSFEGATLREYQRKKEAQAKETQRRKGESKVSWRAYADHIVHALANKVVDAALMHASQVVIEDLSAIAQGPQHKRPKFRPRTNLSKVLNRAQYQKLANVLEYKLAAAGLPPPKLVRAAGTSITCNACGYRDKENRQSQAAFVCLQCGHSENADLHAAKNIAAKHIYWREVGPKVKGKKMTDKHKFENWLAVRRQAVKVD

SEQ ID NO: 99 - protein, 316 aa, synthetic construct; identical to SEQ ID NO: 5

SEQ ID NO: 100 - protein, 322 aa, synthetic construct
MQNILTNPTIKTITFEARPMWETAIHAKKFSGSTSADAVRKENYVKVKEILDNYTRYVINMATQMLVSENLEPTFENLNVLYLKSRDSKSKDDIKRYEKAVKDFKNHVVEVIKKYSTEKQLIKAQGDLFNEKKSCLLNDATAFKLTAEEKELIRLYNGFTGYFSKYFDSLSKTILCGTEYGSISNRIVENMERFFQNKKILEEIQEKYPDLYEKLSFNIKKMTILSCLWQEGIDSYNAVLGDTSNTGINSIINEFRQREKIRVNGLKPLNKIPYAKKKKQIIMQSIDNEEELSLVICESISLVESLIAYAKKHYKNKFRKRR

SEQ ID NO: 101 - protein, 386 aa, synthetic construct
MSGCVRTYKIPVPAQLYSLCDELNRTAGRIYSKTLSLVRKVHKKKGFWLSERSAQKWILRWGEEICVHTHSKQAAIQLYFQALRSYFKASKKTKDALPPYRRKKYLPFIWKNTAIRYREGRIELSLGKKRKPVSVPAKLPEGTVIRQAKLVYEDGSYYLHLAIEVEIASPKDKGKAVGVDLGVIKPVAAVFEDGKAVVYHGGKLNSLLRYRNKRLASFQEAISRTQKGSRRRKKLLRAKKKFLIRIRNQIKDVLHKLTSHFIKTCIERGVSTIVLGDLNGIRDRVRYSKKVNQKIHQWAFRKLAEQLLCKAEAVGIRVIKVSERDTSQECPVCGSKNKTKSRVYRCSNCGFVHHRDIVGAVNILRRYLVSIGATDTVVADLAPPQV

SEQ ID NO: 102 - protein, 397 aa, synthetic construct
MSSENTPTEKTTTNPDTESVEVVETVIVPLRTSERKRERVSTAIDEFQEMCSYMADILPSFSEPEWNLTNTSLYRVLTREFPTEDREVGSKVALAAQRHVTAAFSQHAQTRDGELPSFGERSYFRISNQNIDIVENDSGFGCEVRFIPGEPEWFHISPRPYTRKYLSRIIDGDATYGAGEFHLSDSGDLALHLPVKWDIEMDVADDIEKTMGVDIGIRNLYSAAVVEDGDVKDVSVKSGDEFAHHRHRLREKRAKLSEKGDLNAVKQMRGDEERYTEQVLDTCSKRIVELAAEHAPVVIRLEDLSEYRENNSYVIHDWPFAVLQTKIGNKARAAGIPVEIVSAEDTSTTCRKCGDTNESYRRKDEFSCDNCGYEVHSDVNAAINIAQQDTVEETLNN

SEQ ID NO: 103 - protein, 369 aa, synthetic construct
MTLNDIVNEYNGNTYATNYRDLLAEREGNRTEARKNWKKIENIKDIKQGYLSQVVHIISKMMVEYDAIVVLEDLNMGFMRGRQKIERSVYEQFEKMLIDKLNYYVDKQKDANEAGGLLHALQLTSKFESFKKLGKQSGCLFYIPAWNTSKIDPVTGFVNLFDTRYTNADQARKFFSLFDSIRYNAEKNWFEFAFDYDKFTTKAKGTRTRWTLCTYGTRIRTFRNPAKLNQWDNKEVVLTDEFKKAFADAGIDIHGNLKEAICSLEDKKYLEPLMHLMKLLLQMRNSITNTEVDYLLSPVADKNGSFYDSRVCSYALPKDADANGAYNIARKGLWAIRQIQETPVGERPNLAIKNNEWLNFAQQKPYRDE

SEQ ID NO: 104 - protein, 391 aa, synthetic construct
MYCVVMITYNTKLKFEKQEDFNNLLESLKLNQSAWNIISQERFSMTGGILKILHDKCYYKVKKALPTIPSQFIIKAEQDVIATYKTIKSNKHKLDSPATKKNLSIQLDKRIYKWIDKTHLKITTLNKQAIVSFDVYEKLNELDFSKLKDPKLFFKNGNFYLSLIFDIQLPFIDNNKIVGIDVGLKRLISTSEGNIVKGNEFNGHKRKIRWNKRKFQSKKHSHSARTNRKKLRKKEANFSRNYIHNVTKNVILNTNANILVIEDLSKIKSKNKGKRFNNRLSQLPFFAFRTILTYKAQLYGKKVATVNPAFTSQIDHRGLVGGKRRGCRYYGSDNVVLDADVNAAINIANRYVDNLSLHKHSVLCCKALDGQAAVNQPIVGSSFRQAPIPLG

SEQ ID NO: 105 - DNA, 30 bp, synthetic construct
  source 1..30: mol_type = other DNA
atttcaactcacgccttcacggaaggcgac

SEQ ID NO: 106 - DNA, 30 bp, synthetic construct
  source 1..30: mol_type = other DNA
gtcgccttccgtgaaggcgtgagttgaaat

SEQ ID NO: 107 - DNA, 36 bp, synthetic construct
  source 1..36: mol_type = other DNA
gtcagtagggcagcaaggattagcagccgttgacac

SEQ ID NO: 108 - DNA, 36 bp, synthetic construct
  source 1..36: mol_type = other DNA
gtgtcaacggctgctaatccttgctgccctactgac

SEQ ID NO: 109 - DNA, 36 bp, synthetic construct
  source 1..36: mol_type = other DNA
gcttcagtcggcgcgaattcaggaatcgtgaggcgg

SEQ ID NO: 110 - DNA, 36 bp, synthetic construct
  source 1..36: mol_type = other DNA
ccgcctcacgattcctgaattcgcgccgactgaagc

SEQ ID NO: 111 - DNA, 37 bp, synthetic construct
  source 1..37: mol_type = other DNA
cctgtatgtacatacaaaaataggtgcagaaacagtt

SEQ ID NO: 112 - DNA, 37 bp, synthetic construct
  source 1..37: mol_type = other DNA
aactgtttctgcacctatttttgtatgtacatacagg

SEQ ID NO: 113 - DNA, 36 bp, synthetic construct
  source 1..36: mol_type = other DNA
atctacgaaagtagaaattcttaaagagctttagcc

SEQ ID NO: 114 - DNA, 36 bp, synthetic construct
  source 1..36: mol_type = other DNA
ggctaaagctctttaagaatttctactttcgtagat

SEQ ID NO: 115 - DNA, 19 bp, synthetic construct
  source 1..19: mol_type = other DNA
gctgtatgttacccacatt

SEQ ID NO: 116 - DNA, 19 bp, synthetic construct
  source 1..19: mol_type = other DNA
aatgtgggtaacatacagc

SEQ ID NO: 117 - DNA, 37 bp, synthetic construct
  source 1..37: mol_type = other DNA
agttgtatatacctttcatattaaggtgaattacacc

SEQ ID NO: 118 - DNA, 37 bp, synthetic construct
  source 1..37: mol_type = other DNA
ggtgtaattcaccttaatatgaaaggtatatacaact

SEQ ID NO: 119 - DNA, 36 bp, synthetic construct
  source 1..36: mol_type = other DNA
gttgtaattacattaaaaaccgaaggttgatacaac

SEQ ID NO: 120 - DNA, 36 bp, synthetic construct
  source 1..36: mol_type = other DNA
gttgtatcaaccttcggtttttaatgtaattacaac

SEQ ID NO: 121 - DNA, 42 bp, synthetic construct
  source 1..42: mol_type = other DNA
gtctgctgtatatacccttcattcttaggtgtgtaacaccct

SEQ ID NO: 122 - DNA, 42 bp, synthetic construct
  source 1..42: mol_type = other DNA
agggtgttacacacctaagaatgaagggtatatacagcagac

SEQ ID NO: 123 - DNA, 38 bp, synthetic construct
  source 1..38: mol_type = other DNA
gtcgcgatcgccgtttcagggcaggtcgaattgaaagt

SEQ ID NO: 124 - DNA, 38 bp, synthetic construct
  source 1..38: mol_type = other DNA
actttcaattcgacctgccctgaaacggcgatcgcgac

SEQ ID NO: 125 - DNA, 36 bp, synthetic construct
  source 1..36: mol_type = other DNA
gttttgttaccagtacaaatagaacagttccaaaac

SEQ ID NO: 126 - DNA, 36 bp, synthetic construct
  source 1..36: mol_type = other DNA
gttttggaactgttctatttgtactggtaacaaaac

SEQ ID NO: 127 - DNA, 37 bp, synthetic construct
  source 1..37: mol_type = other DNA
ctttcagcccacccatttctgggaaggcgagggcggc

SEQ ID NO: 128 - DNA, 37 bp, synthetic construct
  source 1..37: mol_type = other DNA
gccgccctcgccttcccagaaatgggtgggctgaaag

SEQ ID NO: 129 - DNA, 36 bp, synthetic construct
  source 1..36: mol_type = other DNA
gccgcagtcgccgcgatttcaggaagcatgaggcgg

SEQ ID NO: 130 - DNA, 36 bp, synthetic construct
  source 1..36: mol_type = other DNA
ccgcctcatgcttcctgaaatcgcggcgactgcggc

SEQ ID NO: 131 - DNA, 37 bp, synthetic construct
  source 1..37: mol_type = other DNA
gtagaaaccaacagggtttctggaaagggattgaaag

SEQ ID NO: 132 - DNA, 37 bp, synthetic construct
  source 1..37: mol_type = other DNA
ctttcaatccctttccagaaaccctgttggtttctac

SEQ ID NO: 133 - DNA, 37 bp, synthetic construct
  source 1..37: mol_type = other DNA
cttccaatcagccgatcgcttaaccagaagcttcacc

SEQ ID NO: 134 - DNA, 37 bp, synthetic construct
  source 1..37: mol_type = other DNA
ggtgaagcttctggttaagcgatcggctgattggaag

SEQ ID NO: 135 - DNA, 20 bp, synthetic construct
  source 1..20: mol_type = other DNA
ccacaaccccaccaggttcc

SEQ ID NO: 136 - DNA, 20 bp, synthetic construct
  source 1..20: mol_type = other DNA
ggaacctggtggggttgtgg

SEQ ID NO: 137 - DNA, 28 bp, synthetic construct
  source 1..28: mol_type = other DNA
ggctgctccgggtgcgcgtggagcgagg

SEQ ID NO: 138 - DNA, 28 bp, synthetic construct
  source 1..28: mol_type = other DNA
cctcgctccacgcgcacccggagcagcc

SEQ ID NO: 139 - DNA, 37 bp, synthetic construct
  source 1..37: mol_type = other DNA
ctttcaatctgctcgttgctgaaaaggcgatcgcgac

SEQ ID NO: 140 - DNA, 37 bp, synthetic construct
  source 1..37: mol_type = other DNA
gtcgcgatcgccttttcagcaacgagcagattgaaag

SEQ ID NO: 141 - DNA, 37 bp, synthetic construct
  source 1..37: mol_type = other DNA
gttgcatccgctttccagcaaccagggcgggtgaaag

SEQ ID NO: 142 - DNA, 37 bp, synthetic construct
  source 1..37: mol_type = other DNA
ctttcacccgccctggttgctggaaagcggatgcaac

SEQ ID NO: 143 - DNA, 37 bp, synthetic construct
  source 1..37: mol_type = other DNA
gtttcaactttccttccagctagaggcgggttgaaag

SEQ ID NO: 144 - DNA, 37 bp, synthetic construct
  source 1..37: mol_type = other DNA
ctttcaacccgcctctagctggaaggaaagttgaaac

SEQ ID NO: 145 - DNA, 37 bp, synthetic construct
  source 1..37: mol_type = other DNA
gttgcactctctgaattaacgacgtgaacgatgcaac

SEQ ID NO: 146 - DNA, 37 bp, synthetic construct
  source 1..37: mol_type = other DNA
gttgcatcgttcacgtcgttaattcagagagtgcaac

SEQ ID NO: 147 - DNA, 36 bp, synthetic construct
  source 1..36: mol_type = other DNA
ctcaaataccaataataaatttctacttttgtagat

SEQ ID NO: 148 - DNA, 36 bp, synthetic construct
  source 1..36: mol_type = other DNA
atctacaaaagtagaaatttattattggtatttgag

SEQ ID NO: 149 - DNA, 37 bp, synthetic construct
  source 1..37: mol_type = other DNA
gtcacaatcagctgtccccatgagggcaggttgaaag

SEQ ID NO: 150 - DNA, 37 bp, synthetic construct
  source 1..37: mol_type = other DNA
ctttcaacctgccctcatggggacagctgattgtgac

SEQ ID NO: 151 - DNA, 28 bp, synthetic construct
  source 1..28: mol_type = other DNA
ggttcctccgctcacgcggagatgcgcc

SEQ ID NO: 152 - DNA, 28 bp, synthetic construct
  source 1..28: mol_type = other DNA
ggcgcatctccgcgtgagcggaggaacc

SEQ ID NO: 153 - DNA, 37 bp, synthetic construct
  source 1..37: mol_type = other DNA
ctttcaacccaccctgttgcgggagggttgatgaaac

SEQ ID NO: 154 - DNA, 37 bp, synthetic construct
  source 1..37: mol_type = other DNA
gtttcatcaaccctcccgcaacagggtgggttgaaag

SEQ ID NO: 155 - DNA, 44 bp, synthetic construct
  source 1..44: mol_type = other DNA
ctttggttgcgatcgccttttcagcagacggcggattgaaagag

SEQ ID NO: 156 - DNA, 44 bp, synthetic construct
  source 1..44: mol_type = other DNA
ctctttcaatccgccgtctgctgaaaaggcgatcgcaaccaaag

SEQ ID NO: 157 - DNA, 37 bp, synthetic construct
  source 1..37: mol_type = other DNA
ctttccactgaccaaatccccgcaaggggacggaaac

SEQ ID NO: 158 - DNA, 37 bp, synthetic construct
  source 1..37: mol_type = other DNA
gtttccgtccccttgcggggatttggtcagtggaaag

SEQ ID NO: 159 - DNA, 39 bp, synthetic construct
  source 1..39: mol_type = other DNA
ggtcgcgatcgccttttcagcaacgagcagattgaaagc

SEQ ID NO: 160 - DNA, 39 bp, synthetic construct
  source 1..39: mol_type = other DNA
gctttcaatctgctcgttgctgaaaaggcgatcgcgacc

SEQ ID NO: 161 - DNA, 36 bp, synthetic construct
  source 1..36: mol_type = other DNA
gtctataaaaggttttaaatttctactattgtagat

SEQ ID NO: 162 - DNA, 36 bp, synthetic construct
  source 1..36: mol_type = other DNA
atctacaatagtagaaatttaaaaccttttatagac

SEQ ID NO: 163 - DNA, 37 bp, synthetic construct
  source 1..37: mol_type = other DNA
gttgcaatcgccttcccagagatgggtgggctgaaag

SEQ ID NO: 164 - DNA, 37 bp, synthetic construct
  source 1..37: mol_type = other DNA
ctttcagcccacccatctctgggaaggcgattgcaac

SEQ ID NO: 165 - DNA, 38 bp, synthetic construct
  source 1..38: mol_type = other DNA
ctttcagccctccctatcactaggagggcgattgcgac

SEQ ID NO: 166 - DNA, 38 bp, synthetic construct
  source 1..38: mol_type = other DNA
gtcgcaatcgccctcctagtgatagggagggctgaaag

SEQ ID NO: 167 - DNA, 37 bp, synthetic construct
  source 1..37: mol_type = other DNA
gtcgcaactgccccttcattcgaagggaggttgaaag

SEQ ID NO: 168 - DNA, 37 bp, synthetic construct
  source 1..37: mol_type = other DNA
ctttcaacctcccttcgaatgaaggggcagttgcgac

SEQ ID NO: 169 - DNA, 36 bp, synthetic construct
  source 1..36: mol_type = other DNA
ggtttgctgacctcgattgttggaggagtgcaaaag

SEQ ID NO: 170 - DNA, 36 bp, synthetic construct
  source 1..36: mol_type = other DNA
cttttgcactcctccaacaatcgaggtcagcaaacc

SEQ ID NO: 171 - DNA, 25 bp, synthetic construct
  source 1..25: mol_type = other DNA
tacaatacctattagggattgaaag

SEQ ID NO: 172 - DNA, 25 bp, synthetic construct
  source 1..25: mol_type = other DNA
ctttcaatccctaataggtattgta

SEQ ID NO: 173 - DNA, 37 bp, synthetic construct
  source 1..37: mol_type = other DNA
gtttcaacacccctcccagcgagaggcgggttgaaag

SEQ ID NO: 174 - DNA, 37 bp, synthetic construct
  source 1..37: mol_type = other DNA
ctttcaacccgcctctcgctgggaggggtgttgaaac

SEQ ID NO: 175 - DNA, 36 bp, synthetic construct
  source 1..36: mol_type = other DNA
ccgcctcatgccgactgaaatcacggggagtgcgac

SEQ ID NO: 176 - DNA, 36 bp, synthetic construct
  source 1..36: mol_type = other DNA
gtcgcactccccgtgatttcagtcggcatgaggcgg

SEQ ID NO: 177 - DNA, 36 bp, synthetic construct
  source 1..36: mol_type = other DNA
gtctgaaggcctaaaggaagctgaatctgctggcac

SEQ ID NO: 178 - DNA, 36 bp, synthetic construct
  source 1..36: mol_type = other DNA
gtgccagcagattcagcttcctttaggccttcagac

SEQ ID NO: 179 - DNA, 36 bp, synthetic construct
  source 1..36: mol_type = other DNA
gtgtcagtcgatcaggagcgagccggctcgctccgc

SEQ ID NO: 180 - DNA, 36 bp, synthetic construct
  source 1..36: mol_type = other DNA
gcggagcgagccggctcgctcctgatcgactgacac

SEQ ID NO: 181 - DNA, 36 bp, synthetic construct
  source 1..36: mol_type = other DNA
gtgccattaccttacgcttctgcaaagcgttcatgc

SEQ ID NO: 182 - DNA, 36 bp, synthetic construct
  source 1..36: mol_type = other DNA
gcatgaacgctttgcagaagcgtaaggtaatggcac

SEQ ID NO: 183 - DNA, 36 bp, synthetic construct
  source 1..36: mol_type = other DNA
gcctgaccgccccgatcaatggtggatagctggcac

SEQ ID NO: 184 - DNA, 36 bp, synthetic construct
  source 1..36: mol_type = other DNA
gtgccagctatccaccattgatcggggcggtcaggc

SEQ ID NO: 185 - DNA, 36 bp, synthetic construct
  source 1..36: mol_type = other DNA
gtcttagggcataaaggaaattgaatttgctggcac

SEQ ID NO: 186 - DNA, 36 bp, synthetic construct
  source 1..36: mol_type = other DNA
gtgccagcaaattcaatttcctttatgccctaagac

SEQ ID NO: 187 - DNA, 36 bp, synthetic construct
  source 1..36: mol_type = other DNA
gtgccagctttcatccaatggccatgaaggaaggac

SEQ ID NO: 188 - DNA, 36 bp, synthetic construct
  source 1..36: mol_type = other DNA
gtccttccttcatggccattggatgaaagctggcac

SEQ ID NO: 189 - DNA, 36 bp, synthetic construct
  source 1..36: mol_type = other DNA
gatcttcctaatcgatcaattgtggatagctggcac

SEQ ID NO: 190 - DNA, 36 bp, synthetic construct
  source 1..36: mol_type = other DNA
gtgccagctatccacaattgatcgattaggaagatc

SEQ ID NO: 191 - DNA, 34 bp, synthetic construct
  source 1..34: mol_type = other DNA
tgtacacatatataatttttactctaacgtaaat

SEQ ID NO: 192 - DNA, 34 bp, synthetic construct
  source 1..34: mol_type = other DNA
atttacgttagagtaaaaattatatatgtgtaca

SEQ ID NO: 193 - DNA, 36 bp, synthetic construct
  source 1..36: mol_type = other DNA
atctacaacagtagaaatttggtggtacctttagac

SEQ ID NO: 194 - DNA, 36 bp, synthetic construct
  source 1..36: mol_type = other DNA
gtctaaaggtaccaccaaatttctactgttgtagat

SEQ ID NO: 195 - DNA, 36 bp, synthetic construct
  source 1..36: mol_type = other DNA
gtctaatacctatataaaatttctacttttgtagat

SEQ ID NO: 196 - DNA, 36 bp, synthetic construct
  source 1..36: mol_type = other DNA
atctacaaaagtagaaattttatataggtattagac

SEQ ID NO: 197 - DNA, 36 bp, synthetic construct
  source 1..36: mol_type = other DNA
accgcagggcctttggatttgaagagcttgtggcgg

SEQ ID NO: 198 - DNA, 36 bp, synthetic construct
  source 1..36: mol_type = other DNA
ccgccacaagctcttcaaatccaaaggccctgcggt

SEQ ID NO: 199 - DNA, 36 bp, synthetic construct
  source 1..36: mol_type = other DNA
gccgcagtcgccgcgactgcggcaatcttgaggcgg

SEQ ID NO: 200 - DNA, 36 bp, synthetic construct
  source 1..36: mol_type = other DNA
ccgcctcaagattgccgcagtcgcggcgactgcggc

SEQ ID NO: 201 - DNA, 36 bp, synthetic construct
  source 1..36: mol_type = other DNA
ccgccacaagctcttcaaagtctcaggccctgcgac

SEQ ID NO: 202 - DNA, 36 bp, synthetic construct
  source 1..36: mol_type = other DNA
gtcgcagggcctgagactttgaagagcttgtggcgg

SEQ ID NO: 203 - DNA, 36 bp, synthetic construct
  source 1..36: mol_type = other DNA
cccgcgctcgatgcgatgtcagcgagactgaggcgg

SEQ ID NO: 204 - DNA, 36 bp, synthetic construct
  source 1..36: mol_type = other DNA
ccgcctcagtctcgctgacatcgcatcgagcgcggg

SEQ ID NO: 205 - DNA, 36 bp, synthetic construct
  source 1..36: mol_type = other DNA
gccgcagtcgccgcgaatccggggggcgtgaggcgg

SEQ ID NO: 206 - DNA, 36 bp, synthetic construct
  source 1..36: mol_type = other DNA
ccgcctcacgccccccggattcgcggcgactgcggc

SEQ ID NO: 207 - DNA, 36 bp, synthetic construct
  source 1..36: mol_type = other DNA
ccgcctgaagctcttcggaaacgcgaggcgttcaac

SEQ ID NO: 208 - DNA, 36 bp, synthetic construct
  source 1..36: mol_type = other DNA
gttgaacgcctcgcgtttccgaagagcttcaggcgg

SEQ ID NO: 209 - DNA, 36 bp, synthetic construct
  source 1..36: mol_type = other DNA
ccgcctcatgctgtctgaaatcgcgccgtgtgcgcc

SEQ ID NO: 210 - DNA, 36 bp, synthetic construct
  source 1..36: mol_type = other DNA
ggcgcacacggcgcgatttcagacagcatgaggcgg

SEQ ID NO: 211 - DNA, 36 bp, synthetic construct
  source 1..36: mol_type = other DNA
gttccttccgcagtgatttctaaaagcttgaggcgg

SEQ ID NO: 212 - DNA, 36 bp, synthetic construct
  source 1..36: mol_type = other DNA
ccgcctcaagcttttagaaatcactgcggaaggaac

SEQ ID NO: 213 - DNA, 34 bp, synthetic construct
  source 1..34: mol_type = other DNA
gccgcagtcgccgcgattccgggggaagtgaggc

SEQ ID NO: 214 - DNA, 34 bp, synthetic construct
  source 1..34: mol_type = other DNA
gcctcacttcccccggaatcgcggcgactgcggc

SEQ ID NO: 215 - DNA, 36 bp, synthetic construct
  source 1..36: mol_type = other DNA
ccgcctcacgctcttcgaaaccgcgggatgtgcgac

SEQ ID NO: 216 - DNA, 36 bp, synthetic construct
  source 1..36: mol_type = other DNA
gtcgcacatcccgcggtttcgaagagcgtgaggcgg

SEQ ID NO: 217 - DNA, 36 bp, synthetic construct
  source 1..36: mol_type = other DNA
gtcccccccgtagcgatctcaggaaacgtgaggcgg

SEQ ID NO: 218 - DNA, 36 bp, synthetic construct
  source 1..36: mol_type = other DNA
ccgcctcacgtttcctgagatcgctacgggggggac

SEQ ID NO: 219 - DNA, 36 bp, synthetic construct
  source 1..36: mol_type = other DNA
ggtgccgacgtcggcaccaacctgatcgacggacac

SEQ ID NO: 220 - DNA, 36 bp, synthetic construct
  source 1..36: mol_type = other DNA
gtgtccgtcgatcaggttggtgccgacgtcggcacc

SEQ ID NO: 221 - DNA, 36 bp, synthetic construct; identical to SEQ ID NO: 219
  source 1..36: mol_type = other DNA

SEQ ID NO: 222 - DNA, 36 bp, synthetic construct; identical to SEQ ID NO: 220
  source 1..36: mol_type = other DNA

SEQ ID NO: 223 - DNA, 36 bp, synthetic construct
  source 1..36: mol_type = other DNA
gtggcagaagtcggaaccgatctgatcaactgacac

SEQ ID NO: 224 - DNA, 36 bp, synthetic construct
  source 1..36: mol_type = other DNA
gtgtcagttgatcagatcggttccgacttctgccac

SEQ ID NO: 225 - DNA, 36 bp, synthetic construct
  source 1..36: mol_type = other DNA
ggctcctacgttgaaactagcctgatcggctgacac

SEQ ID NO: 226 - DNA, 36 bp, synthetic construct
  source 1..36: mol_type = other DNA
gtgtcagccgatcaggctagtttcaacgtaggagcc

SEQ ID NO: 227 - DNA, 40 bp, synthetic construct
  source 1..40: mol_type = other DNA
cccgttgcaattcatggggagtggagtggtatcatgaaag

SEQ ID NO: 228 - DNA, 40 bp, synthetic construct
  source 1..40: mol_type = other DNA
ctttcatgataccactccactccccatgaattgcaacggg

SEQ ID NO: 229 - DNA, 21 bp, synthetic construct
  source 1..21: mol_type = other DNA
tcaattctatagtagatcatc

SEQ ID NO: 230 - DNA, 21 bp, synthetic construct
  source 1..21: mol_type = other DNA
gatgatctactatagaattga

SEQ ID NO: 231 - DNA, 21 bp, synthetic construct
  source 1..21: mol_type = other DNA
gatgatctactatagaattga

SEQ ID NO: 232 - DNA, 21 bp, synthetic construct
  source 1..21: mol_type = other DNA
tcaattctatagtagatcatc

SEQ ID NO: 233 - DNA, 36 bp, synthetic construct
  source 1..36: mol_type = other DNA
gtccccactcgctggggacattaattgaatggaaac

SEQ ID NO: 234 - DNA, 36 bp, synthetic construct
  source 1..36: mol_type = other DNA
gtttccattcaattaatgtccccagcgagtggggac

SEQ ID NO: 235 - DNA, 36 bp, synthetic construct
  source 1..36: mol_type = other DNA
gcctaagagcataatgaaatttctactgttgtagat

SEQ ID NO: 236 - DNA, 36 bp, synthetic construct
  source 1..36: mol_type = other DNA
atctacaacagtagaaatttcattatgctcttaggc

SEQ ID NO: 237 - DNA, 30 bp, synthetic construct
  source 1..30: mol_type = other DNA
gcttcaacccctcaagggtgcatctgaaac

SEQ ID NO: 238 - DNA, 30 bp, synthetic construct
  source 1..30: mol_type = other DNA
gtttcagatgcacccttgaggggttgaagc

SEQ ID NO: 239 - DNA, 30 bp, synthetic construct
  source 1..30: mol_type = other DNA
gcttcaaacccacaagggttcatctgaaac

SEQ ID NO: 240 - DNA, 30 bp, synthetic construct
  source 1..30: mol_type = other DNA
gtttcagatgaacccttgtgggtttgaagc

SEQ ID NO: 241 - DNA, 30 bp, synthetic construct
  source 1..30: mol_type = other DNA
gcttcaacccctcaagggttcatctggaac

SEQ ID NO: 242 - DNA, 30 bp, synthetic construct
  source 1..30: mol_type = other DNA
gttccagatgaacccttgaggggttgaagc

SEQ ID NO: 243 - DNA, 30 bp, synthetic construct
  source 1..30: mol_type = other DNA
gtttcagccgaacccttgaggggttgcagt

SEQ ID NO: 244 - DNA, 30 bp, synthetic construct
  source 1..30: mol_type = other DNA
actgcaacccctcaagggttcggctgaaac

SEQ ID NO: 245 - DNA, 35 bp, synthetic construct
  source 1..35: mol_type = other DNA
atctacgagagtagaaattaacatatactgtcaga

SEQ ID NO: 246 - DNA, 35 bp, synthetic construct
  source 1..35: mol_type = other DNA
tctgacagtatatgttaatttctactctcgtagat

SEQ ID NO: 247 - DNA, 33 bp, synthetic construct
  source 1..33: mol_type = other DNA
gtcgctccccgctcggggagcgtgagttgaaac

SEQ ID NO: 248 - DNA, 33 bp, synthetic construct
  source 1..33: mol_type = other DNA
gtttcaactcacgctccccgagcggggagcgac

SEQ ID NO: 249 - DNA, 36 bp, synthetic construct
  source 1..36: mol_type = other DNA
gtcacagctaccttccaaataggcacctgctttagc

SEQ ID NO: 250 - DNA, 36 bp, synthetic construct
  source 1..36: mol_type = other DNA
gctaaagcaggtgcctatttggaaggtagctgtgac

SEQ ID NO: 251 - DNA, 36 bp, synthetic construct; identical to SEQ ID NO: 113
  source 1..36: mol_type = other DNA

SEQ ID NO: 252 - DNA, 36 bp, synthetic construct; identical to SEQ ID NO: 114
  source 1..36: mol_type = other DNA

SEQ ID NO: 253 - DNA, 36 bp, synthetic construct
  source 1..36: mol_type = other DNA
gttttgaagtgttaaataatatctactattgtagat

SEQ ID NO: 254 - DNA, 36 bp, synthetic construct
  source 1..36: mol_type = other DNA
atctacaatagtagatattatttaacacttcaaaac

SEQ ID NO: 255 - DNA, 30 bp, synthetic construct
  source 1..30: mol_type = other DNA
ctttcaatcccttataggtaggctaaaaac

SEQ ID NO: 256 - DNA, 30 bp, synthetic construct
  source 1..30: mol_type = other DNA
gtttttagcctacctataagggattgaaag

SEQ ID NO: 257 - DNA, 33 bp, synthetic construct
  source 1..33: mol_type = other DNA
tccgtttcaaacgaacccatgaggggttgaagc

SEQ ID NO: 258 - DNA, 33 bp, synthetic construct
  source 1..33: mol_type = other DNA
gcttcaacccctcatgggttcgtttgaaacgga

SEQ ID NO: 259 - DNA, 36 bp, synthetic construct
  source 1..36: mol_type = other DNA
ggctataaagccattttaatttctactattgtagat

SEQ ID NO: 260 - DNA, 36 bp, synthetic construct
  source 1..36: mol_type = other DNA
atctacaatagtagaaattaaaatggctttatagcc

SEQ ID NO: 261 - DNA, 36 bp, synthetic construct
  source 1..36: mol_type = other DNA
ggtgatatagtaactggtctgttccagcacttcacc

SEQ ID NO: 262 - DNA, 36 bp, synthetic construct
  source 1..36: mol_type = other DNA
ggtgaagtgctggaacagaccagttactatatcacc

SEQ ID NO: 263 - DNA, 97 bp, synthetic construct
  source 1..97: mol_type = other DNA
tttcagcagcacaaggtcggtgacatcttcatcacgagctacggcagcctggatcgcctgaaagttttgctgacctgtttggcgatttttgtcgtac

SEQ ID NO: 264 - DNA, 337 bp, synthetic construct
  source 1..337: mol_type = other DNA
ttcctgaaaaatattctcgacccattgatagtagaacaggcgcggacaatagatgaagttgtgcaagcgccgcactggcagaggcggctgggcctcgagtgcggtttgggtttggaccggcgcgtcgctcattacgcctacattgggacatcgtccttcacttgctcatgattcagccaccgccgaagccgttcgttgttgatggcctcgcaccgcctccactggtattcccgatcattcacggtcttccaaacaccgggaccggaggcgtagtcgaatcgcccagccgaatcggtttccagtcgcgctcgcccgaaaggggcgatttcatgtcggt

SEQ ID NO: 265 - DNA, 121 bp, synthetic construct
  source 1..121: mol_type = other DNA
tttcaattgtatactgtaagaacactttttgatggatagggtgtccatggtaacgtatttaagctttggttcatgtggtgtttttaagcttcccatgttctgtgtgggatgtttttggtgt

SEQ ID NO: 266 - DNA, 97 bp, synthetic construct
  source 1..97: mol_type = other DNA
tatagaaacattcctaccctagaagacgagaatttaagtttaataaaaagctagctcttcaaatctcagactataagctagctcttcttgctctctt

SEQ ID NO: 267 - DNA, 164 bp, synthetic construct
  source 1..164: mol_type = other DNA
ctactatgtgcccgcatgctcttatgtatcgatccaaggggatccttatgagctgaggatcaatgttgttaatattataatggccataatacttagtaagttaaacataccctgaggttcgggtttcattgggcttggctttggccaagctctattcccttggc

SEQ ID NO: 268 - DNA, 186 bp, synthetic construct
  source 1..186: mol_type = other DNA
cacatgcggggcagacgtaaacgcggtctttgagtttcaggccgctatgcacatgtccgcaggcgtggcaggtcttggaactcgggtagaatcgatccgcgatgcggaattcaatcccgatgacccctgccttgcgccgtagtttctcacggagggaataaaagcgctgctgcgccacgaccttcg

SEQ ID NO: 269 - DNA, 36 bp, synthetic construct
  source 1..36: mol_type = other DNA
atctacaacagtagaaattaaaacgggtattaaaac

SEQ ID NO: 270 - DNA, 36 bp, synthetic construct
  source 1..36: mol_type = other DNA
gttttaatacccgttttaatttctactgttgtagat

SEQ ID NO: 271 - DNA, 24 bp, synthetic construct
  source 1..24: mol_type = other DNA
gaatccatacaatggaattgaaag

SEQ ID NO: 272 - DNA, 24 bp, synthetic construct
  source 1..24: mol_type = other DNA
ctttcaattccattgtatggattc

SEQ ID NO: 273 - DNA, 36 bp, synthetic construct
  source 1..36: mol_type = other DNA
acttcaccctgcactccgttcgtcacctgctcacgc

SEQ ID NO: 274 - DNA, 36 bp, synthetic construct
  source 1..36: mol_type = other DNA
gcgtgagcaggtgacgaacggagtgcagggtgaagt

SEQ ID NO: 275 - DNA, 25 bp, synthetic construct
  source 1..25: mol_type = other DNA
ctttcaattctatagtagatcatct

SEQ ID NO: 276 - DNA, 25 bp, synthetic construct
  source 1..25: mol_type = other DNA
agatgatctactatagaattgaaag

SEQ ID NO: 277 - DNA, 306 bp, synthetic construct
  source 1..306: mol_type = other DNA
atgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttttggggaatataa

SEQ ID NO: 278 - DNA, 26 bp, synthetic construct
  source 1..26: mol_type = other DNA
attctaaagatctttgacagctagct

SEQ ID NO: 279 - DNA, 26 bp, synthetic construct
  source 1..26: mol_type = other DNA
tccagaaatcatccttagcgaaagct

SEQ ID NO: 280 - DNA, 37 bp, synthetic construct
  source 1..37: mol_type = other DNA
ctaaggatgatttctggaacgtctcattttcgccaga

SEQ ID NO: 281 - DNA, 44 bp, synthetic construct
  source 1..44: mol_type = other DNA
gtcaaagatctttagaatagtgtgactctagtagagagcgttca

SEQ ID NO: 282 - DNA, 20 bp, synthetic construct
  source 1..20: mol_type = other DNA
gggcacgggcagcttgcccg

SEQ ID NO: 283 - DNA, 20 bp, synthetic construct
  source 1..20: mol_type = other DNA
ctaaagaggagaaaggatct

SEQ ID NO: 284 - DNA, 20 bp, synthetic construct
  source 1..20: mol_type = other DNA
tgggccctcaaataaaacga

SEQ ID NO: 285 - DNA, 20 bp, synthetic construct
  source 1..20: mol_type = other DNA
atgctttgctaaagtgaggt

SEQ ID NO: 286 - DNA, 20 bp, synthetic construct
  source 1..20: mol_type = other DNA
gatgcattattatgtgtcaa

(19) United States
(12) Patent Application Publication
(10) Pub. No.: US 2026/0185067 A1
(43) Pub. Date: Jul. 2, 2026

(54) DEVELOPMENT OF DNA TARGETED GENE EDITING TOOL

(71) Applicant: SHANGHAI GENEMAGIC BIOSCIENCES CO., LTD., Shanghai (CN)

(72) Inventors: Haibo ZHOU, Shanghai (CN); Zhengzheng XU, Shanghai (CN); Qi MA, Shanghai (CN)

(73) Assignee: SHANGHAI GENEMAGIC BIOSCIENCES CO., LTD., Shanghai (CN)

(21) Appl. No.: 18/863,818

(22) PCT Filed: May 8, 2023
(86) PCT No.: PCT/CN2023/092784

§ 371 Date: Nov. 7, 2024

(30) Foreign Application Priority Data
May 7, 2022 (WO) PCT/CN2022/091550

(51) Int. Cl. C12N 9/22; C12N 15/11; C12Q 1/34; C12Q 1/6823

(52) U.S. Cl. CPC C12N 9/226; C12N 15/111; C12Q 1/34; C12Q 1/6823; C12N 2310/20; C12N 2800/22; G01N 2333/922

(57) ABSTRACT

The present application relates to a Cas12 protein having an amino acid sequence set forth in any one of SEQ ID NOs: 1 to 104, or a functional fragment thereof, or having an amino acid sequence set forth in any one of SEQ ID NOs: 1 to 104 with one or more amino acid substitutions, insertions, and/or deletions, and a CRISPR-Cas system comprising the Cas12 protein, and use thereof.

### RELATED APPLICATIONS

This application claims priority of the international application No. PCT/CN2022/091550 which entitled “development of DNA targeted gene editing tools” and filed on May 7, 2022. The entire content of the application is incorporated herein by reference.

### THE SUBMITTED SEQUENCE LISTING FILE

The entire content of the following XML file is incorporated herein by reference in its entirety: Sequence Listing in Computer-Readable Format (CRF) (name: TFG00801PCT-sequence listing.xml, date: 20230506, size: 316 KB).

### TECHNICAL FIELD

This application relates to the newly discovered Cas12 proteins, which belong to the field of gene editing biology.

### BACKGROUND TECHNIQUE

The CRISPR-Cas system is a type of acquired immune system in prokaryote. It functions as an adaptive immune mechanism in microorganisms such as bacteria and archaeato resist viruses and other foreign nucleic acids. The CRISPR-Cas immune response mainly includes three stages: adaptation stage, expression and processing stage, and interference stage. Similar to other immune mechanisms, CRISPR-Cas systems develop in the context of constant competition with mobile genetic elements, which results in extreme diversity in Cas protein sequences and CRISPR-Cas locus structures.

Since 2011, based on the genetic composition of the CRISPR-Cas system, the architecture of the genetic loci, and clustering methods such as sequence similarity, the CRISPR-Cas systems can currently be categorized into two major classes. Among them, the Class1 system contains an effector module composed of multiple Cas proteins, some of which form crRNA-binding complexes that mediate pre-crRNA processing and interference through additional Cas proteins. The Class 2 system contains a single Cas effector protein with a multifunctional domain binding region, which is capable of binding crRNA and participating in all activities required for interference. In some variants, it also involves the maturation process of pre-crRNA. Currently, Class 2 CRISPR-Cas systems are mainly divided into three subtypes: type II (such as Cas9), type V (such as Cas12), and type VI (such as Cas13). Among them, type VI effector Cas proteins mainly target RNA, while type II and type V subtypes mainly target DNA.

Now, researchers study more on Cas12 proteins and develop the gene editing tools related to Crispr-Cas12. However, there are still some shortcomings. On the one hand, many of the Cas12 proteins discovered so far are relatively large. When using the CRISPR-Cas12 system, delivery media are usually required to deliver the relevant plasmids. The delivery media usually used is retrovirus, adenovirus or adeno-associated viruses. Due to their limited loading capacity, for example, the loading capacity of AAV delivery vector which is commonly used is only 4.7 kb, which is not conducive to package the CRISPR-Cas related tools having large molecular weights into AAV. On the other hand, the Cas12 protein exhibits a strong DNA sequence preference (PAM) when targeting DNA, which limits the use of Cas12 protein to a certain extent. Some researchers tried to obtain PAM-independent Cas12 protein, but this will reduce the enzyme cleavage activity.

Therefore, there is still a need to find cas12 proteins with low molecular weight, easy delivery, or PAM-independent.

### CONTENTS OF THE INVENTION

For the shortcomings of existing Cas 12 proteins, this application provides a novel cas12 protein family, which is not only small in molecular size but also has good gene editing capabilities.

The technical problem solved by this disclosure is to find candidate CRISPR-Cas12 proteins and systems with more novel DNA cleavage active domains (such as RuvC, Cas12 superfamily, InsQ superfamily, etc.); to verify the activity of the candidate CRISPR-Cas12 proteins and these systems; and to obtain a variety of new Cas12 proteins finally.

This disclosure achieves the following technical effects:

(1) An analytical method has been developed for the rapid screening of novel Cas12 family proteins. This method enables the analysis of CRIPSR arrays within newly updated prokaryotic microbial DNA sequences and metagenomic sequences, as well as the selection of related effector proteins.

(2) New Cas12 family members are screened, and to the application scope of CRISPR-Cas12 is expanded. On the one hand, most of the novel candidate Cas12 family members screened in this application are less than 400 amino acids in length, some of them are less than 300 amino acids, and some of them are even less than 200 amino acids. The length of these is significantly shorter than the Cas12 family members in the prior art. Due to their low molecular weight, these candidate Cas12 proteins can be efficiently packaged by delivery vectors such as adeno-associated viruses, thereby enabling the diagnosis and treatment of related diseases such as neurodegenerative diseases. On the other hand, although some candidate Cas 12 proteins have large molecular weights, they have different PAM preferences and expand the toolbox for nucleic acid detection. In addition, these candidate proteins can also be used for carrying out research on breeding and stress tolerance in the plant field, and can be used for modifying the related engineering bacteria in the microbial field.

(3) In addition to utilizing the known RuvC domain of Cas12 proteins for screening, the method provided in this application also includes conserved domains with DNA-cleaving activity from other types of proteins, thereby providing the possibility of screening for new Cas12 proteins. And due to the identification of these new functional domains of these novel Cas12 proteins, new ideas and possibilities are provided for further modification of Cas12 proteins.

In one aspect of the present disclosure, Cas12 proteins are provided.

In a preferred embodiment, the Cas12 protein comprises the amino acid sequence shown in any one of SEQ ID NO: 1-104 or a functional fragment thereof, or the Cas12 protein comprises the amino acid sequence of any one of SEQ ID NO: 1-104 with conservative amino acid substitution at one or more residues, or the Cas12 protein comprises the amino acid sequence of any one from SEQ ID NO: 1 to 104 with one or more amino acid substitutions, insertions, and/or deletions.

In a preferred embodiment, the Cas12 protein is a fragment of the amino acid sequence shown in any one of SEQ ID NO: 1-104, or is a fragment of the amino acid sequence shown in any one of SEQ ID NO: 1-104 having one or more amino acid substitutions, insertions, and/or deletions.

In a preferred embodiment, the Cas12 protein comprises the amino acid sequence of SEQ ID NO: 13, 25, 31, 59, 61, 62, 63, 66, or 67; or comprises a fragment of the amino acid sequence shown in SEQ ID NO: 13, 25, 31, 59, 61, 62, 63, 66, or 67; or comprises a mutant of the amino acid sequence shown in SEQ ID NO: 13, 25, 31, 59, 61, 62, 63, 66, or 67.

In a preferred embodiment, the Cas12 protein comprises the amino acid sequence of SEQ ID NO: 25, 31, 62, 63, or 66; or comprises a fragment of the amino acid sequence shown in SEQ ID NO: 25, 31, 62, 63, or 66; or comprises a mutant of the amino acid sequence shown in SEQ ID NO: 25, 31, 62, 63, or 66.

In a preferred embodiment, the DNA cleavage activity of the Cas 12 protein is retained.

In a preferred embodiment, the Cas12 protein has the activity for gene knock-in, gene knock-out, or gene modification of on DNA.

In a preferred embodiment, the Cas12 protein has RuvC domain, Cas12 superfamily domain and/or InsQ superfamily domain, and at least one amino acid of RuvC domain, Cas12 superfamily domain and/or InsQ superfamily domain has been further modified or engineered to reduce or eliminate its DNA cleavage activity, which results in a dCas12 (dead Cas12) with reduced or abolished DNA cleavage activity.

In a preferred embodiment, the Cas12 protein has DNA editing activity, preferably, at least one of its RuvC domain, Cas12 superfamily domain and InsQ superfamily domain is further modified or engineered to reduce or eliminate its DNA cleavage activity, which results in a dCas12 (dead Cas12) with reduced or abolished DNA cleavage activity.

In a preferred embodiment, the Cas12 protein is fused with one or more heterologous functional domains.

In a preferred embodiment, the fusion occurs at the N-terminal, C-terminal or internal part of the Cas12 protein.

In a preferred embodiment, the heterologous functional domain is capable of cleaving one or more target sequence, or modifying the transcription or translation of the target sequence.

In a preferred embodiment, the one or more heterologous functional domains have the following activities: deaminase such as cytidine deaminase and deoxyadenosine deaminase, methylase, demethylase, transcriptional activation, transcriptional repression, nuclease, single-stranded RNA cleavage, double-stranded RNA cleavage, single-stranded DNA cleavage, double-stranded DNA cleavage, DNA or RNA ligase, reporter protein, detection protein, localization signal, or any combination thereof.

In a preferred embodiment, the Cas12 protein comprises RuvC domain, Cas12 superfamily domain, and/or InsQ superfamily domain; preferably, the Cas12 protein comprises cas12k domain, cas12b domain, RuvC_1 domain, and/or OrfB/InsQ domain.

In a preferred embodiment, the amino acid substitution, insertion, and/or deletion in the Cas12 proteins of the present application includes performing substitution, insertion, and/or deletion in the RuvC domain, the Cas12 superfamily domain, and/or the InsQ superfamily domain. Preferably, it includes substitution, insertion, and/or deletion in the cas12k domain, cas12b domain, RuvC_1 domain, and/or OrfB/InsQ domain.

In a preferred embodiment, after substitution, insertion, and/or deletion of one or more amino acids, the Cas12 protein of the present application exhibits reduced or eliminated activity for gene knock-in, gene knock-out, or modification on DNA.

In another aspect of the present disclosure, a nucleic acid molecule is provided, which includes a nucleotide sequence encoding the above-mentioned Cas12 protein of the present application.

In a preferred embodiment, the nucleic acid molecule is used for codon optimization for the expression in a specific host cell.

In a preferred embodiment, the host cell is prokaryotic cell or eukaryotic cell, preferably is human cell.

In a preferred embodiment, the host cell is prokaryotic cell or eukaryotic cell, preferably is animal cell, plant cell, or microbial cell.

In a preferred embodiment, the nucleic acid molecule comprises a promoter operably linked to the nucleotide sequence encoding Cas12, which is a constitutive promoter, an inducible promoter, a synthetic promoter, a tissue-specific promoter, a chimeric promoter, or development-specific promoters.

In another aspect of the present disclosure, an expression vector is provided, which comprises the above-mentioned nucleic acid molecule, and expresses the above-mentioned amino acid sequence or nucleotide sequence in the form of DNA, RNA or protein.

In another aspect of the present disclosure, an expression vector is provided, which comprises the above-mentioned nucleic acid molecule of the present application.

In a preferred embodiment, the expression vector further comprises crRNA sequence and/or tracr RNA sequence.

In a preferred embodiment, the expression vector further comprises a regulatory element that regulates the nucleic acid molecule, a regulatory element that regulates the crRNA sequence, and/or a regulatory element that regulates the tracr RNA sequence.

In a preferred embodiment, the expression vector is viral vector, nanoparticle, liposome nanoparticle (LNP), cationic polymer (e.g. PEI), liposome, exosome, virus-like particle (VLP), microvesicle or gene gun.

In a preferred embodiment, the expression vector is adeno-associated virus (AAV), adenovirus, recombinant adeno-associated virus (rAAV), lentivirus, retrovirus, herpes simplex virus, oncolytic virus, etc.

In another aspect of the present disclosure, a delivery system is provided, which includes (1) the above-mentioned expression vector, or the above-mentioned Cas12 protein; and (2) a delivery vector.

In a preferred embodiment, the delivery vehicle is liposome nanoparticle (LNP), cationic polymer (e.g. PEI), virus-like particle (VLP), nanoparticle, liposome, exosome, microvesicle or gene gun, etc.

In another aspect of the present disclosure, a CRISPR-Cas system is provided, which includes: (1) the Cas 12 protein or the nucleic acid molecule described in the present application, or a derivative or functional fragment thereof; (2) a gRNA sequence for targeting to a target DNA or a target sequence.

In a preferred embodiment, the functional fragment of the Cas12 protein is a fragment of the amino acid shown in any one from SEQ ID NO: 1 to 104 with at least one amino acid deletion and retains the function of Cas12 protein. Preferably, the functional fragment of the Cas12 protein comprises at least one amino acid insertion, deletion, and/or substitution (for example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 residues) based on any one of the amino acid sequences shown in SEQ ID NOs: 1 to 104. Preferably, the said substitution is conservative substitution and still retains the function of the said Cas12 protein.

In a preferred embodiment, the derivative of the Cas12 protein is a protein that has more than 70%, 80%, 85%, 90%, 91%, 92%, 93%, 94%, 95%, 96%, 97%, 98%, or 99% identity to any one of the proteins or their functional fragments shown from SEQ ID NO: 1 to 104. Preferably, the derivative of the Cas12 protein is a protein that has at least 1, 2, 3, 4, 5, 6, 7, 8, 9, or 10 amino acids insertion, deletion, and/or substitution based on the amino acid sequence shown in any one from SEQ ID NOs: 1 to 104.

In a preferred embodiment, wherein a portion of the said gRNA sequence comprises a direct repeat (DR) sequence, a trans-acting CRISPR RNA (tracrRNA) and a spacer sequence targeting a target RNA segment (Spacer sequence).

In a preferred embodiment, the gRNA sequence comprises a direct repeat (DR) sequence, a trans-acting CRISPR RNA (tracrRNA) and a spacer sequence targeting a target sequence.

In a preferred embodiment, the other part of the gRNA sequence comprises a direct repeat (DR) sequence and a spacer region sequence targeting a target RNA segment (Spacer sequence).

In a preferred embodiment, the DR sequence is the sequence shown in Table 1; the tracrRNA sequence is the sequence shown in Table 2; wherein the spacer sequence has 10-60 nucleotides, preferably has 15-25 nucleotides, more preferably has 19-21 nucleotides.

In a preferred embodiment, the DR sequence is the sequence shown in any one from SEQ ID NO: 105 to 262 or the sequence shown in any one from SEQ ID NO: 269 to 276.

In a preferred embodiment, the tracrRNA sequence is the sequence shown in any one from SEQ ID NO: 263 to 268.

In a preferred embodiment, the spacer sequence has 10-50 nucleotides, preferably has 15-25 nucleotides, more preferably has 20 nucleotides.

In a preferred embodiment, the DR sequence may be a derivative corresponding to any of the following, wherein the derivative: (i) has one or more (e.g., 1, 2, 3, 4, 5, 6, 7, 8, 9, or 10) nucleotide additions, deletions, or substitutions compared to any of the sequences shown in Table 1; (ii) has at least 20%, 30%, 40%, 50%, 60%, 70%, 80%, 90%, 95%, or 97% sequence identity to any of the sequences shown in Table 1; (iii) hybridizes to any of the sequences shown in Table 1 or hybridizes to any of the sequences described in (i) and (ii) under stringent conditions; or (iv) is a complement of any one of (i)-(iii), provided that the derivative is not any of the sequences shown in Table 1, and the derivative encodes RNA, or itself is RNA, which maintains essentially the same secondary structure as any RNA encoded by SEQ ID NO: 105-262.

In a preferred embodiment, the DR sequence is any one of the following derivatives from (i) to (iv), wherein, the derivative (i) has one or more (e.g., 1, 2, 3, 4, 5, 6, 7, 8, 9, or 10) nucleotide additions, deletions, or substitutions compared to any of the sequences shown from SEQ ID NO: 105 to 262 or from SEQ ID NO: 269 to 276; the derivative (ii) has at least 20%, 30%, 40%, 50%, 60%, 70%, 80%, 90%, 95%, or 97% sequence identity to any of the sequences from SEQ ID NO: 105 to 262 or from SEQ ID NO: 269 to 276; the derivative (iii) hybridizes to any of the sequences shown from SEQ ID NO: 105 to 262 or from SEQ ID NO: 269 to 276 or hybridizes to any of the sequences described in (i) and (ii) under stringent conditions; or the derivative (iv) is a complement of any one of the derivatives (i)-(iii), provided that the derivative is not any of the sequences shown from SEQ ID NO: 105 to 262 or from SEQ ID NO: 269 to 276, and the derivative encodes RNA or itself is RNA, which maintains essentially the same secondary structure as any RNA encoded by SEQ ID NO: 105-262 or SEQ ID NO: 269-276.

In a preferred embodiment, the tracrRNA sequence is the sequence shown in Table 2; the sequence includes a segment of pairing bases that are complementary to the described DR sequence in reverse. They can generally form at least 6, 8, 10, or 12 pairing bases, the said pairing bases are either continuous or spaced.

In a preferred embodiment, the tracrRNA sequence described includes a segment of pairing bases that are complementary to the described DR sequence in reverse. Preferably, the tracrRNA sequence and the DR sequence form at least 6, 8, 10, or 12 pairing bases, the said pairing bases are either continuous or spaced. Preferably the tracrRNA sequence is the sequence shown in any one from SEQ ID NO: 263 to 268.

In a preferred embodiment, the tracrRNA sequence may be a derivative corresponding to any of the following, wherein the derivative: (i) has one or more (e.g., 1, 2, 3, 4, 5, 6, 7, 8, 9, or 10) nucleotide additions, deletions, or substitutions compared to any of the sequences shown in Table 2; (ii) has at least 20%, 30%, 40%, 50%, 60%, 70%, 80%, 90%, 95% or 97% sequence identity to any of the sequences shown in Table 2; (iii) hybridizes to any of the sequences shown in Table 2 or hybridizes to any of the sequences described in (i) and (ii) under stringent conditions; or (iv) is a complement of any one of (i)-(iii), provided that the derivative is not any of the sequences shown in Table 2, and the derivative encodes RNA or itself is RNA, which maintains essentially the same secondary structure as any RNA encoded by any one of SEQ ID NO: 263-268.

In a preferred embodiment, the tracrRNA is any one of the following derivatives from (i) to (iv), wherein, the derivative (i) has one or more (e.g., 1, 2, 3, 4, 5, 6, 7, 8, 9, 10, 11, 12, 13, 14, 15, 16, 17, 18, 19 or 20) nucleotide additions, deletions, or substitutions compared to any one of the sequences shown from SEQ ID NO: 263 to 268; the derivative (ii) has at least 20%, 30%, 40%, 50%, 60%, 70%, 80%, 90%, 95% or 97% sequence identity to any one of the sequences shown from SEQ ID NO: 263 to 268; the derivative (iii) hybridizes to any of the sequences shown from SEQ ID NO: 263 to 268 or hybridizes to any of the sequences described in (i) and (ii) under stringent conditions; or the derivative (iv) is a complement of any one of the derivatives (i)-(iii), provided that the derivative is not any one of the sequences shown from SEQ ID NO: 263 to 268, and the derivative encodes RNA or itself is RNA, which maintains essentially the same secondary structure as any RNA encoded by any one of SEQ ID NO: 263-268.

In a preferred embodiment, the CRISPR-Cas system further includes: (3) target RNA.

In a preferred embodiment, the CRISPR-Cas system can trigger the cleavage, sequence insertion or deletion, single base editing, sequence modification (including epigenetic modification), sequence change or degradation of the target DNA sequence.

In a preferred embodiment, the target DNA is double-stranded DNA, single-stranded DNA, double-stranded circular DNA, or single-stranded circular DNA.

In a preferred embodiment, the system is capable of delivering epigenetic modifiers or transcriptional or translational activation or repression signals at or near the target sequence.

In another aspect of the present disclosure, a cell is provided, it comprises the above-mentioned Cas12 protein, nucleic acid molecule, expression vector, delivery system or CRISPR-Cas system.

In a preferred embodiment, the cell is prokaryotic cell or eukaryotic cell, preferably is human cell.

In another aspect of the present disclosure, a method is provided for degrading, cutting, changing or modifying a target DNA in a target cell, wherein the method comprises the use of the Cas12 protein disclosed herein, the nucleic acid molecule disclosed herein, the expression vector disclosed herein, the delivery vector disclosed herein, or the CRISPR-Cas system disclosed herein.

In a preferred embodiment, the target cell is prokaryotic cell or eukaryotic cell, preferably is human cell.

In a preferred embodiment, the target cell is a prokaryotic cell or a eukaryotic cell, preferably is animal cell, plant cell, or microbial cell.

In a preferred embodiment, the target cell is ex vivo cell, in vitro cell or in vivo cell.

In another aspect of the present disclosure, a target DNA detection method is provided, the target DNA is detected by the ues of the Cas12 protein or its derivative or its functional fragment described in the application, the Cas12 protein or derivative or its functional fragment expressed by the nucleic acid molecules described in the application, the Cas12 protein or its derivative or functional fragment expressed by the expression vector described in this application, or the CRISPR-Cas system described in this application.

In a preferred embodiment, the target DNA detection method also uses sgRNA targeting the target DNA and a reporter detection molecule. Using the Cas12 protein, its derivative or functional fragment described in the present application, the Cas12 protein, its derivative or functional fragment expressed by the nucleic acid molecules described in the present application, the Cas12 protein, its derivatives or functional fragments expressed by the expression vector described in this application, or the CRISPR-Cas system described in this application, it would bind to the target DNA and then activate collateral DNA cleavage activity of the Cas12 protein. The activation results in the cleavage of the reporter molecule and allows for the detection of the signal emitted by the reporter molecule.

### FIGURES

FIG. 1 shows the results of DZ356 protein cutting the endogenous gene TYR in 293T cell line. It can be seen that two cleavages are observed near sg1 (the first sgRNA targeting TYR) when DZ356 protein is co-transfected with guide RNA (sgMix). In contrast, no cleavage is detected in control group px377 (a tool plasmid that has the same plasmid backbone as DZ356 but lacks the DZ356 protein) and sgMix, which indicate that no cutting occurs.

FIG. 2 shows the results of the DZ738 protein cutting the endogenous gene TYR in 293T cell line. FIG. 2A is the result of the treatment groups. It can be seen that multiple cleavages are observed near both sg1 (the first sgRNA targeting TYR) and sg2 (the second sgRNA targeting TYR) in treatment groups. Furthermore, indel mutations are also detected near sg2 in treatment group 1. These indicate that the candidate protein DZ738 has carried out cleavage near the sgRNA and resulted in large fragment deletions. FIG. 2B shows the results of the DZ738 protein cutting the endogenous gene TYR in control groups. Both control groups show no mutations or cleavages near sg1 and sg2, indicating that the background of the control groups are clean.

FIG. 3 shows the results of the treatment and control groups for the DZ761 protein cutting the endogenous gene TYR in 293T cell line. It can be seen that multiple cleavages can be observed near sg1 in treatment group, while no deletions occurred in control group.

FIG. 4 shows the results of the DZ837 protein cutting the endogenous gene TYR in 293T cell line. FIG. 4A shows that large-scale cleavages (deletions) are observed near both sg1 and sg2 in treatment groups. Further more, indel mutations are also detected in treatment group 2. The background of the control groups (px262 is an empty plasmid without sgRNA, and px377 is an empty plasmid without DZ837.) are clean. These indicate DZ837 has the ability to cleave endogenous genes.

FIG. 4B shows that large-scale cleavages (deletions) are observed near both sg1 and sg2 in treatment group. Further more, indel mutations are also detected in treatment group 2. The background of the control group (px262 is an empty plasmid without sgRNA, and px377 is an empty plasmid without DZ837.) is clean. These indicate that DZ837 has the ability to cleave endogenous genes.

FIG. 5 shows the results of the positive control LbCas12 cutting the endogenous gene TYR in 293T cell line. It can be seen that large-scale cleavages (deletions) and indel mutations are observed near both sg1 and sg2 in treatment groups, while the background of the control groups are clean. These further indicate that the positive control protein has the ability to cleave endogenous genes.

FIG. 6 shows the flow cytometric analysis of candidate protein DZ402 to target and knock out the mCherry fluorescent protein in 293T cells. The abscissa represents the intensity of green light, the ordinate represents the intensity of red light, and the Q2 group represents the group of cells that simultaneously express the red fluorescence protein mCherry and the green fluorescent protein EGFP. Compared with the control group, the sgRNA in treatment groups successfully knock out the mCherry fluorescent protein, resulting in a decrease in the proportion of red and green double-positive cells in Q2.

FIG. 7 shows the flow cytometric analysis of candidate protein DZ428 to target and knock out mCherry fluorescent protein in 293T cells. The abscissa represents the intensity of green light, the ordinate represents the intensity of red light, and the Q2 group represents the group of cells that simultaneously express the red fluorescence protein mCherry and the green fluorescent protein EGFP. Compared with the control group, the sgRNA in treatment groups successfully knock out mCherry fluorescent protein, resulting in a decrease in the proportion of red and green double-positive cells in Q2.

FIG. 8 shows the flow cytometric analysis of candidate protein DZ738 to target and knock out the mCherry fluorescent protein in 293T cells. The abscissa represents the intensity of green light, the ordinate represents the intensity of red light, and the Q2 group represents the group of cells that simultaneously express the red fluorescence protein mCherry and the green fluorescent protein EGFP. Compared with the control group, the sgRNA in treatment group successfully knocks out mCherry fluorescent protein, resulting in a decrease in the proportion of red and green double-positive cells in Q2.

FIG. 9 shows the flow cytometric analysis of candidate protein DZ761 to target and knock out the mCherry fluorescent protein in 293T cells. The abscissa represents the intensity of green light, the ordinate represents the intensity of red light, and the Q2 group represents the group of cells that simultaneously express the red fluorescence protein mCherry and the green fluorescent protein EGFP. Compared with the control group, the sgRNA in treatment group successfully knocks out mCherry fluorescent protein, resulting in a decrease in the proportion of red and green double-positive cells in Q2.

FIG. 10 shows a summary bar chart showing the residual rates of red fluorescence protein mCherry and the green fluorescent protein EGFP in cell populations after targeted knockdown using the candidate proteins DZ402, DZ428, DZ738 and DZ761 in 293T cells. It can be seen that compared with the control group, several sgRNAs in treatment groups of these proteins successfully knock out mCherry fluorescent protein, resulting in a decrease in the proportion of red and green double-positive cells in Q2. Among them, the cleavage effects of the DZ738 and DZ761 proteins are the most significant.

FIG. 11 shows the experimental results showing the colony cloning situation of E. coli with positive protein SpCas9 in both the treatment and control groups on solid culture medium plates during the forward screening system experiment. It can be seen that the number of E. coli cloned colonies in treatment group corresponding to the SpCas9 protein is significantly greater than that in control group, indicating that the forward screening system constructed is reliable.

FIG. 12 shows the experimental results of forward screening of DNA enzyme digestion by using the candidate proteins DZ402, DZ428, DZ832, DZ833 and DZ836. Among them, FIG. 12A, FIG. 12B, FIG. 12C, FIG. 12D, and FIG. 12E respectively shows the colony growth of candidate proteins DZ402, DZ428, DZ832, DZ833, and DZ836. FIG. 12F shows a statistical comparison of the number of Escherichia coli clones in treatment group and the control group within the forward screening system for DZ402, DZ428, DZ832, DZ833, or DZ836. It can be seen that the number of E. coli cloned colonies in treatment group corresponding to these proteins is significantly greater than that in control group.

FIG. 13 shows the PAM screening results of the candidate protein DZ428. It can be seen that there is a significant difference of the E. coli cloned colonies in treatment group and control group. Through second-generation sequencing and analysis, it is found that the modified protein has a significant base sequence preference at the 5′ end and the potential motif is 5′-NNNNNT-Spacer-3′.

FIG. 14 shows the PAM screening results of the candidate protein DZ832. It can be seen that there is a significant difference of the E. coli cloned colonies in treatment group and control group. Through second-generation sequencing and analysis, it is found that the modified protein has a significant base sequence preference at the 5′ end and the potential motif is 5′-NNNTNN-Spacer-3′.

FIG. 15 shows the evolutionary lineage of candidate proteins and the known cas12 protein family. Among them, N1 to N7 represent the entirely new cas12 families we have screened, which generally have a relatively low protein size with most of them containing fewer than 400 amino acids.

### DETAILS

This application provides novel cas12 protein families. The minimum protein length of the novel Cas12 family members screened in this application is composed of 105 amino acids, and a large number of them are composed of about 200 or 300 amino acids. Such Cas12 proteins are significantly smaller than the existing Cas12 proteins. These low-molecular-weight Cas12 protein can be well packaged through delivery vectors such as adeno-associated viruses, thereby enabling the diagnosis and treatment of related diseases.

Moreover, the novel Cas12 family proteins screened in this application also have different PAM preferences, expanding the toolkit of nucleic acid detection. In addition, these candidate proteins can also be used for carrying out research on breeding and stress tolerance in the plant field, and can be used for modifying the related engineering bacteria in the microbial field.

In addition, most of the candidate proteins in this application, especially those having less than 400 amino acids, are completely new families (FIG. 15). It is inconsistent with the currently known evolutionary branch of cas12 family proteins. It not only expands the members of the cas12 protein family, but also deepens our understanding of the ultra-small cas12 protein family from evolutionary perspective.

The embodiments of the present invention will be described in detail below in conjunction with examples. Those skilled in the art will understand that the following examples are only used to illustrate the present invention and should not be considered as limiting the scope of the present invention. If the specific conditions are not specified in the examples, the conditions should be carried out according to the conventional conditions or the conditions recommended by the manufacturer. Cell lines, reagents, or instruments without indicating the manufacturer are all conventional products that can be purchased commercially.

As used in the specification, a noun without quantifier embellishment may mean one/a kind or many/many kinds. As used in the claims, a noun without quantifier embellishment may mean one/a kind of or more than one/more than one kind if it is used with the word “comprise/include”.

Although this disclosure supports the definitions which only indicate alternatives or supports the limitation using “and/or”, the term “or” used in the claims means “and/or” unless it is explicitly stated that it only refers to alternatives or the alternatives are mutually exclusive. As used herein, “another/others” may mean at least two or more.

In this whole application, the term “about” is used to indicate a value which includes device error, the inherent variation in the method used to determine the value, or the inherent variation existing in the study subjects. Such inherent variation may be a variation of +10%.

In this whole application, unless otherwise stated, nucleotide sequences are listed from 5′ to 3′, and amino acid sequences are listed from the N-terminal to C-terminal.

Through the following detailed description, other objects, features and advantages of the present invention will become apparent. However, it should be understood that while certain preferred embodiments of the invention are indicated, the detailed description and specific examples are only given by the way of illustration. Various changes and modifications in the spirit and scope of the invention will become apparent to those skilled in the art based on this detailed description.

### Definition

NCBI (https://www.ncbi.nlm.nih.gov/) refers to the U.S. National Center for Biological Information. It is a public database for the world. Those skilled in the field use the nucleic acid database provided by this database to download prokaryotes genome and proteome-related databases. Additionally, they can also use the blast comparison software provided by the database to perform sequence alignment and analysis.

IMG (https://img.jgi.doe.gov/) refers to the integrated microbial genome database and is a representative of the new generation genome databases. It can not only completely include the content of existing databases, but also provide more complete services of data upload, annotation, and analysis. Sequencing data is stored in the IMG/M database. This data can be downloaded for pure culture bacterial sequencing genomes, metagenomes, metagenomic assembled genomes, and single-cell sequencing genomes.

The term “CRISPR” (cluster regularly interspaced short palindromic repeats) is a locus of a nucleic acid cutting system, such as the locus used by bacteria to destroy foreign DNA (Horvath and Barrangou, 2010, Science (327): 167-170; WO 2007/025097). The CRISPR locus contains short variable DNA sequences (called ‘spacer’) and short direct repeats sequences (DR sequences). In the prokaryotes the CRISPR mainly refer to a string of DNA sequences in bacteria and archaea, including direct repeat (DR) region and non-repeating spacer region. The term “CRIPSR-Cas system” includes not only the CRISPR array locus, but also associated effector proteins which is Cas proteins. They constitute the immune system of prokaryotes (bacteria and archaea) to resist the foreign viruses invasion.

The term “RuvC domain” refers to the cleavage domain of an endogenous nuclease that cleaves DNA. Currently it contains three types as RuvCI, RuvCII and RuvCIII, which is an important DNA-cleaving domains of the Cas12 protein.

The term “ABE system” is the abbreviation of adenine base editors, namely purine base conversion technology that can achieve single base changes from A/T to G/C. The enzyme most commonly used is adar enzyme (adenosine deaminases acting on RNA, an adenosine deaminase acting on RNA). It achieves the mutation from A/T to G/C mainly by deaminating adenine into inosine, which will be seen as G when decode DNA or RNA. Due to the cell's insensitivity to the excision repair of inosine, this mutation can maintain a high purity of the product.

The term “CBE system” is the abbreviation of cytidine base editor, namely pyrimidine base conversion technology. Currently the tools are asBE1, BE2 and BE3. Among them, BE3 has the highest efficiency and therefore is widely used in the fields of gene therapy, animal model production and functional gene screening.

The term “protospacer adjacent motif” refers to the fact that the effector protein of the CRISPR-Cas system often exhibits preference to the protospacer adjacent motif (PAM) and/or protospacer flanking sequence (PFS) of the target sequence when the effector protein target the target nucleic acid sequence (target sequence).

The term “nucleic acid” means a polynucleotide and includes single- or double-stranded polymers of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to refer to single- or double-stranded RNA and/or DNA and/or RNA-DNA polymers, which optionally contains synthetic, non-natural or altered nucleotide bases. Nucleotides (usually found in their 5′-monophosphate form) are represented by single-letter abbreviations as follows: “A” stands for adenosine or deoxyadenosine (for RNA or DNA respectively), “C” stands for cytidine or deoxycytidine, “G” represents guanosine or deoxyguanosine, “U” represents uridine, “T” represents deoxythymidine, “R” represents purine (A or G), “Y” represents pyrimidine (C or T), “K” represents G or T, “H” represents A or C or T, “I” represents inosine, and “N” represents any nucleotide.

The term “endogenous” refers to sequences or other molecules naturally existing in cell or organism.

The terms “knock-out,” “cleavage,” and “gene editing” are used interchangeably herein. It means that the DNA sequence of the cell is partially or completely ineffective through gene editing with a gene editing tool (such as the Crispr-Cas system); for example, such a DNA sequence may have encoded an amino acid sequence before being knocked out, or may already have a regulatory function (e.g. promoter).

“Domain” means a continuous stretch of nucleotides (which may be RNA, DNA and/or combined RNA-DNA sequences) or amino acids.

The term “conserved domain” or “motif” refers to a set of polynucleotides or amino acids that are conserved at a specific position along aligned sequences of evolutionarily related proteins. Although amino acids at other positions can vary among homologous proteins, amino acids which are highly conserved at a specific position are the essential amino acids to the structure, stability, or activity of the protein. Because they are identified by high conservation in aligned sequences of protein homolog families, they can be used as identifiers or “signatures” to determine whether a protein with a new sequence belongs to a previously identified protein family.

A “codon-modified gene” or “codon-biased gene” or “codon-optimized gene” is a gene whose codon usage frequency is designed to mimic the codon usage frequency preferred the host cell.

An “optimized” polynucleotide is a sequence that has been optimized to improve expression in a particular heterologous host cell.

A “plant-optimized nucleotide sequence” is a nucleotide sequence optimized for expression in plants (in particular for increased expression in plants). Plant-optimized nucleotide sequences include codon-optimized genes. One or more plant-preferred codons can be used to improve expression. Through the modifying the nucleosides of the encoded proteins (such as Cas endonucleases disclosed herein) plant-preferred nucleotide sequences can be synthesized. See, for example, Campbell and Gowri (1990) Plant Physiol. 92:1-11 discussion of host-preferred codon usage.

A “promoter” is a DNA region that participates in RNA polymerase to recognize and bind to other proteins so as to initiate transcription. The promoter sequence consists of proximal element and more distal upstream element, the upstream element is often called as enhancer. An “enhancer” is a DNA sequence that can stimulate the activity of a promoter and may be an intrinsic element of the promoter or a heterologous element inserted to enhance the level or tissue specificity of the promoter.

The promoter may be entirely derived from a natural gene, or may be composed of different elements derived from different promoters in nature, and/or contains synthetic DNA segments. Those skilled in the art will understand that different promoters may direct the expression of genes in different tissues or cell types, or at different developmental stages, or in response to different environmental conditions. It is further recognized that, since the exact boundaries of regulatory sequences are not fully defined in most cases, some variant DNA segments may have the same promoter activity.

“Host” refers to an organism or cell into that has been introduced with heterologous components (polynucleotides, polypeptides, other molecules, cells). As used herein, “host cell” refers to a eukaryotic cell, a prokaryotic cell (eg, a bacterial or archaeal cell) in vivo or in vitro, or a cell from a multicellular organism cultured as a unicellular entity (e.g., cell line), which has been introduced with a heterologous polynucleotide or polypeptide has been introduced. In some embodiments, the cells are selected from the group consisting of primitive cells, bacterial cells, eukaryotic cells, eukaryotic unicellular organisms, somatic cells, germ cells, stem cells, plant cells, algae cells, animal cells, invertebrate cells, vertebrate cells, fish cells, frog cells, bird cells, insect cells, mammalian cells, pig cells, bovine cells, goat cells, sheep cells, rodent cells, rat cells, mouse cells, non-human primate cells, and human cells. In some cases, the cells are in vitro cells. In some cases, the cells are in vivo cells.

In one embodiment, the prokaryotic cell is Escherichia cell, or Bacillus cell, or Lactobacillus cell, or Corynebacterium cell, or yeast cell (Saccharomyces, Candida or Pichia). In a further embodiment, the cell is Escherichia coli cell, or Bacillus subtilis cell, or Lactobacillus acidophilus cell, or Corynebacterium glutamicum cell, or Pasteurian Pichia pastoris cell.

In one embodiment, the prokaryotic cell is E. coli K12 cell or E. coli B cell.

In one embodiment, the prokaryotic cell is E. coli K12 cell, wherein it has the genotype as follows: thi-1, ompT, pyrF, acnA, aceA, icd (parental strain) and genotype: thi-1, ompT, pyrF, ndh, acnA, aceA, icd (modified strain), in which the polypeptide encoded by the acnA gene contains the S68G mutation, the polypeptide encoded by the aceA gene contains the S522G mutation, and the polypeptide encoded by the icd gene contains the D398E and D410E mutations. Furthermore, the parental and modified strains lack the following e14 phage genes: ymfD, ymfE, lit, intE, xisE, ymfl, ymfJ, cohE, croE, ymfL, ymfM, owe, ymfR, bee, jayE, ymfQ, stfP, tfaP, tfaE, stfE, pinE, mcrA.

As used herein, a “eukaryotic cell” may be a mammalian cell, including human cells (e.g., human primary cells, established human cell lines, or cells in vivo) and non-human mammalian cells (e.g., cells derived from non-human primate (e.g. monkeys), cows/bulls/cattle, sheep, goats, pigs, horses, dogs, cats, rodents (e.g. rabbits, rats, hamsters, etc.).

As used herein, a “host cell” may be derived from fish (e.g. salmon), bird (e.g. poultry including chicks, ducks, geese), reptiles, shellfish (e.g. oysters, clams, lobsters, shrimps), insects, worms, yeast, etc. “Host cells” can also be from plants, such as monocots or dicots. The plant may be a food crop such as barley, cassava, cotton, peanut, corn, millet, oil palm, potato, legume, rapeseed or canola, rice, rye, sorghum, soybean, sugarcane, sugar beet, sunflower and wheat. The plant may be a cereal (eg barley, corn, millet, rice, rye, sorghum and wheat). The plants may be tubers (e.g., cassava and potatoes). In some embodiments, the plant may be a sugar crop (e.g., sugar beet and sugar cane). The plants may be oily crops (e.g., soybeans, peanuts, rapeseed or canola, sunflowers and oil palm). The plant may be a fiber crop (e.g., cotton). The plant may be a tree such as a peach or nectarine tree, an apple tree, a pear tree, an almond tree, a walnut tree, a pistachio tree, a citrus tree (e.g., orange, grapefruit or lemon tree), grass, vegetable, fruit or algae. The plant may be a plant of solanum; a plant of brassica; a plant of the lactuca; a plant of the spinacia; a plant of the capsicum; cotton, tobacco, asparagus, carrot, cabbage, broccoli, cauliflower, tomatoes, eggplants, peppers, lettuce, spinach, strawberries, blueberries, raspberries, blackberries, grapes, coffee, cocoa, etc.

The term “recombination” refers to the artificial combination of two originally separate sequence segments, such as manipulating isolated nucleic acid segments combination through chemical synthesis or genetic engineering techniques.

The term “plasmid” refers to a linear or circular extrachromosomal element that usually carries a portion of gene. The element is typically in the form of double-stranded DNA. Such element may be autonomously replicating sequences, genome integrating sequences, bacteriophages, or nucleotide sequences derived from any source, which can be in the form of single or double stranded DNA or RNA and can be linear or circular form. Many of these nucleotide sequences have been linked or reorganized into unique constructs which are capable of introducing the interested polynucleotide of interest into cells.

The term “expression kit” refers to a specific vector containing a gene and having extragenic elements that allow expression of the gene in a host.

The terms “recombinant DNA molecule,” “recombinant DNA construct,” “expression construct,” “construct,” and “recombinant construct” are used interchangeably herein. Recombinant DNA constructs contain nucleic acid sequences, such as the artificial combination of regulatory sequence and coding sequence not all found in nature. For example, a recombinant DNA construct may contain regulatory sequence and coding sequence derived from different sources, or regulatory sequence and coding sequence derived from the same source but arranged in a manner different from their natural occurrence. This construct can be used alone or in combination with a vector. If a vector is used, the choice of vector depends on the method to be used to introduce the vector into the host cell as is well known to those skilled in the art. For example, plasmid vectors can be used. The skilled person is well aware of the genetic elements that must be present on the vector for successful transformation, selection and propagation in the host cells. The skilled in the art will also recognize that different independent transformation events may result in different expression levels and patterns (Jones et al. (1985) EMBO J [European Molecular Biology Organization] 4:2411-2418; De Almeida et. al., (1989) Mol Gen Genetics [Molecular and General Genetics] 218:78-86), therefore multiple events are typically screened to obtain genetic element which has the desired expression levels and patterns. Such screening involve standard molecular biology assays, biochemistry assays and other assays, including DNA blot analysis, Northern analysis of mRNA expression, PCR, quantitative real-time PCR (qPCR), reverse transcription PCR (RT-PCR), immunoblot analysis of protein expression, enzyme assays or activity assays, and/or phenotypic analysis.

The term “heterologous” refers to the difference between the original environment, location, or composition of a particular polynucleotide or polypeptide sequence and its current environment, location, or composition. Non-limiting examples include taxonomically derived differences (e.g., if a polynucleotide sequence obtained from Zea mays is inserted into the genome of an Oryza sativa plant or into the genome of a different variant or cultivar of Zea mays, the polynucleotide sequence is heterologous; or a polynucleotide sequence obtained from a bacterium is introduced into a plant cell, the polynucleotide sequence is heterologous) or sequence differences (e.g., a polynucleotide sequence obtained from Zea mays is isolated, modified and then reintroduced into Zea mays). As used herein, “heterologous” with respect to a sequence may mean that the sequence is derived from different species, variant, exotic species, or, if derived from the same species, the sequence is obtained from a substantial modification of the natural form appeared in the composition and/or genomic locus under deliberate human intervention. For example, a promoter operably linked to a heterologous polynucleotide is from a different species with the species deriving the polynucleotide, or, if from the same/similar species, one or both are substantially obtained from their original form and/or the modified genomic locus, or the promoter is not a native promoter operably linked to the polynucleotide. Alternatively, one or more regulatory regions and/or polynucleotides provided herein may be synthesized in their entirety. In another example, the target polynucleotide for cleavage by the Cas endonuclease may belong to a different organism with the Cas endonuclease. In another example, the Cas endonuclease and guide RNA can be introduced into the target polynucleotide together with an additional polynucleotide that serves as a template or donor for insertion into the target polynucleotide, wherein the additional polynucleotide is heterologous to the target polynucleotide and/or the Cas endonuclease.

The term “expression” refers to the production of a functional end product (e.g., mRNA, guide RNA, or protein) in a precursor or mature form.

The terms “Cas protein”, “Cas endonuclease” and “Cas enzyme” are used interchangeably herein, which refer to the polypeptide encoded by the Cas (CRISPR-related) gene. Cas proteins include proteins encoded by genes in the cas locus, and include adapting molecules as well as interfering molecules. Interfering molecules of bacterial adaptive immune complexes include endonucleases. Cas endonucleases described herein contain one or more nuclease domains. Cas endonucleases include, but are not limited to: the novel Cas12 proteins disclosed herein, Cas9 proteins, Cpf1 (Cas12) proteins, C2c1 proteins, C2c2 proteins, C2c3 proteins, Cas3, Cas3-HD, Cas5, Cas7, Cas8, Cas10, Cas13, Cas14, or combinations or complexes of these. In this application, the Cas 12 protein may include one or more RuvC nuclease domains, InsQ superfamily domains, or Cas 12 superfamily domains.

In this application, Cas protein is further defined as also comprising functional fragments or derivatives of native Cas protein, for example, The Cas protein is a protein that at least 50, 50 to 100, at least 100, 100 to 150, at least 150, 150 to 200, at least 200, 200 to 250, at least 250, 250 to 300, at least 300, 300 to 350, at least 350, 350 to 400, at least 400, 400 to 450, at least 500 or more than 500 consecutive amino acids of the Cas protein have at least 50%, 50% to 55%, at least 55%, 55% to 60%, at least 60%, 60% to 65%, at least 65%, 65% to 70%, at least 70%, 70% to 75%, at least 75%, 75% to 80%, at least 80%, 80% to 85%, at least 85%, 85% to 90%, at least 90%, 90% to 95%, at least 95%, 95% to 96%, at least 96%, 96% to 97%, at least 97%, 97% to 98%, at least 98%, 98% to 99%, at least 99%, 99% to 100% or 100% sequence identity with the native Cas protein and retains at least a portion of the activity of the native sequence.

Methods for culturing prokaryotic cells are known to those skilled in the art (see, for example, Riesenberg, D., et al., Curr. Opin. Biotechnol. 2 (1991) 380-384). Any method can be used for cultivation. In one embodiment, the culture is a form of batch culture, fed-batch culture, perfusion cultivating, semi-continuous culture, or culture with total or partial cell retention.

The term “conservative amino acid substitutions” refers to the interchangeability of amino acid residues with similar side chains in proteins. For example, the group of amino acids with aliphatic side chains consists of glycine, alanine, valine, leucine, and isoleucine; the group of amino acids with aliphatic-hydroxyl side chains consists of serine and threonine; a group of amino acids with amide-containing side chains consists of asparagine and glutamine; a group of amino acids with aromatic side chains consists of phenylalanine, tyrosine, and tryptophan; a group of amino acids with basic side chains consists of lysine, arginine, and histidine; a group of amino acids with acidic side chains consists of glutamic acid and aspartic acid; and a group of amino acids with sulfur-containing side chains consists of cysteine and methionine. Examples for the conservative amino acid substitution groups are: valine-leucine-isoleucine, phenylalanine-tyrosine, lysine-arginine, alanine-valine-glycine, and asparagine-glutamine.

A nucleic acid or polypeptide has a certain percentage of “sequence identity” with another nucleic acid or polypeptide, which means that the percentage of bases or amino acids are the same when compared, and the bases or amino acids are in the same relative position when the two sequences are compared. Sequence identity can be determined in many different ways. To determine sequence identity, sequences can be aligned using a variety of convenient methods and computer programs (e.g., BLAST, T-COFFEE, MUSCLE, MAFF T, etc.), which are available through the World Wide Web including ncbi.nl m.nili.gov/BLAST, ebi.ac.uk/Tools/msa/tcoffee/, ebi.ac.uk/Tools/msa/muscle/, mafft.cbrc.jp/alignment/software/. See, e.g., Alts chul et al. (1990), J. Mol. Bioi. 215:403-10.

The DNA sequence that “encodes” a specific RNA is the DNA nucleotide sequence that is transcribed into RNA. A DNA polynucleotide may encode an RNA (mRNA) that can convert into a protein (so both DNA and mRNA encode a protein), or a DNA polynucleotide may encode an RNA that is not translated into a protein (e.g., tRNA, rRNA, microRNA (miRNA), “non-coding” RNA (ncRNA), guide RNA, etc.).

“Protein coding sequence” or “sequence encoding a specific protein or polypeptide” means a nucleotide sequence capable of being transcribed into mRNA (in the case of DNA) and translated into a polypeptide (in the case of mRNA) in vitro or in vivo under the control of appropriate regulatory sequences.

The terms “DNA regulatory sequence”, “control element” and “regulatory element” are used interchangeably herein to refer to a sequence to control the transcription and translation, such as promoter, enhancer, polyadenylation signal, terminator, protein degradation signal, etc. The sequence provides and/or regulates the transcription of non-encoding sequence (e.g., guide RNA) or encodes sequence (e.g., RNA-guided endonucleases, GeoCas9 polypeptides, GeoCas9 fusion polypeptide, etc.), and/or regulate translation of the encoded polypeptide.

A “promoter” refers to a DNA regulatory region capable of binding RNA polymerase and initiating the transcription of downstream (3′ direction) coding or non-coding sequences. For purposes of this disclosure, a promoter sequence is bound by the transcription start site at its 3′ end and extends to upstream (5′ direction), and contain the minimum necessary bases or elements to initiate transcription at a detectable level above background. Within the promoter sequence will be found the transcription start site, as well as the protein binding domain responsible for binding RNA polymerase. Eukaryotic promoters will usually, but not always, contain a “TATA” box and a “CAT” box. Various promoters, including inducible promoters, can be used to drive expression of the various vectors of the present disclosure.

The term “cleavage” means the cleavage of the covalent backbone of a target nucleic acid molecule (e.g., RNA, DNA). Cleavage can be initiated by a variety of methods, which include but not limited to enzymatic or chemical hydrolysis of phosphodiester bonds. Both single-stranded and double-stranded cleavages are possible, and double-stranded cleavage can occur as a result of two distinct single-stranded cleavage events.

“Nuclease” and “endonuclease” are used interchangeably herein to mean an enzyme having catalytic activity for nucleic acid cleavage (e.g., ribonuclease activity (ribonucleic acid cleavage), deoxyribonuclease activity (deoxyribonucleic acid cleavage), etc.).

“Cleaving domain” or “active domain” or “nuclease domain” of a nuclease means a polypeptide sequence or domain of a nuclease that has catalytic activity for nucleic acid cleavage. The cleavage domain may be contained in a single polypeptide chain, or the cleavage activity may result from the association of two (or more) polypeptides. A single nuclease domain may consist of more than one discrete amino acids segment within a given polypeptide.

The terms “dead Cas12” and “dcas12” can be used interchangeably in this article and have the same meaning, which include cas12 proteins with reduced DNA cleavage activity and cas12 proteins with eliminated DNA cleavage activity. For example, by modifying or transforming at least one amino acid of the cas12 protein screened in this application (such as amino acid insertion, deletion, substitution, etc.), or by truncating the sequence, the protein with reduced or eliminated DNA cleavage activity will be obtained compared to the parent cas12 protein. Preferably, it may involve the modification or alteration of at least one amino acid, or truncation of the sequence, within its RuvC domain, Cas 12 superfamily domain, and/or InsQ superfamily domain (particularly the cas12k domain, cas12b domain, RuvC_1 domain, and/or OrfB/InsQ domain).

### CRISPR System

CRISPR (Clustered Regularly Interspaced Short Palindromic Repeats)/Cas9 (CRISPR-associated protein 9)-mediated RNA editing is becoming a promising tool for disease diagnosis and treatment, plant breeding, etc.

CRISPR is a DNA locus that contains short repeats of a base sequence. Each repeat is followed by a short segment of “spacer DNA” from previous exposure to the virus. CRISPR is found in approximately 40% of sequenced eubacterial genomes and 90% of sequenced archaea. CRISPR is often associated with Cas gene that encode CRISPR-related protein. The CRISPR/Cas system is a prokaryotic immune system that confers resistance to foreign genetic elements (such as plasmids and phages) and provides a form of acquired immunity. CRISPR spacers recognize and silence these foreign genetic elements in eukaryotic organisms (e.g., RNAi).

CRISPR repeats are 24 to 48 base pairs in size. They usually show some twofold symmetry, which means the secondary structures such as hairpins are formed, but they are not true palindromes. Repeated sequences are separated by the spacer having similar length. Some CRISPR spacer sequences accurately match with the sequences from plasmids and phages, although some spacers match with the prokaryote genomes. New spacers can be rapidly added in response to phage infection. crRNA refers to an abbreviation of CRISPR RNA, which contains the DR sequence and the spacer sequence targeting the target region.

Guide RNA (gRNA) refers to a piece of RNA used by the CRISPR-Cas system to guide effector proteins to act at specific sites on nucleic acids. The CRISPR-Cas12 system includes a combination of crRNA and tracrRNA or only contains crRNA, which is used for CRISPR-Cas 12 targeting to DNA.

The gRNA sequence described in this application mainly includes direct repeat (DR) sequence and spacer sequence targeting the target sequence. For the candidate proteins having tracrRNA, the gRNA corresponding to the protein also includes trans-acting CRISPR RNA (tracrRNA).

### Nuclease

Cas nuclease. CRISPR-associated (Cas) genes are often associated with CRISPR repeat-spacer arrays. As of 2013, more than forty different Cas protein families have been described. Among these protein families, Cas1 is ubiquitous in different CRISPR/Cas systems. The specific combination of Cas gene and repeat structure has been used to define eight CRISPR subtypes (Ecoli, Ypest, Nmeni, Dvulg, Tneap, Hmari, Apern, and Mtube), some of which is associated with other gene module encoding repeat-associated mysterious protein (RAMP). More than one CRISPR subtypes can exist in a single genome. The sporadic distribution of CRISPR/Cas subtypes suggests that this system has undergone horizontal gene transfer during microbial evolution.

The exogenous DNA is apparently processed into small elements (about 30 base pairs in length) by the proteins encoded by the Cas genes, and then these units are inserted, in some manner, into the CRISPR locus proximal to the leader sequence. RNA from the CRISPR locus is constitutively expressed and processed by Cas proteins into small RNAs composed of individual exogenous sequence elements with flanking repeats. RNA directs other Cas protein to silence exogenous genetic elements at the RNA or DNA level.

### EXAMPLES

### Example 1: Screening of Novel Cas12 Proteins

Download the sequences of all bacterial, archaeal genomes and metagenomes from NCBI and IMG as of July 2021, and use CRISPR array identification software (such as Pilercr) to identify the CRISPR array region; search for the 6 adjacent ones upstream and downstream of the CRISPR array region Protein target domain analysis.

The amino acid sequence numbers, DNA cleavage domain types and other information of the obtained candidate proteins are shown in Table 3. The screened candidate proteins have domains such as RuvC domain, InsQ, or Cas12 superfamily.

### Example 2: Functional Verification of Novel Candidate Cas12 Protein to Knock Down 293T Endogenous Gene

In order to verify the ability of the candidate protein screened in Example 1 to cleave endogenous genes, we selected proteins such as DZ356, DZ738, DZ761, DZ837 from the candidate proteins (Table 3) as well as the positive control LbCas12 protein to cleave the endogenous genes (TYR) of 293T cells.

First, two sgRNAs (containing crRNA and tracrRNA) were randomly designed for the TYR endogenous gene, and constructed the corresponding plasmid, namely sg1 (targeting spacer sequence is atgctttgctaaagtgaggt (SEQ ID NO: 285)) and sg2 (targeting spacer sequence is gatgcattattatgtgtcaa (SEQ ID NO: 286)).

Then, the sgRNA and candidate protein were transiently transfected into the 293T cell line (HEK-293T, commercially available). After 48 hours, the top 15% positive cells were sorted by flow cytometry for deep-seq library construction and sequencing.

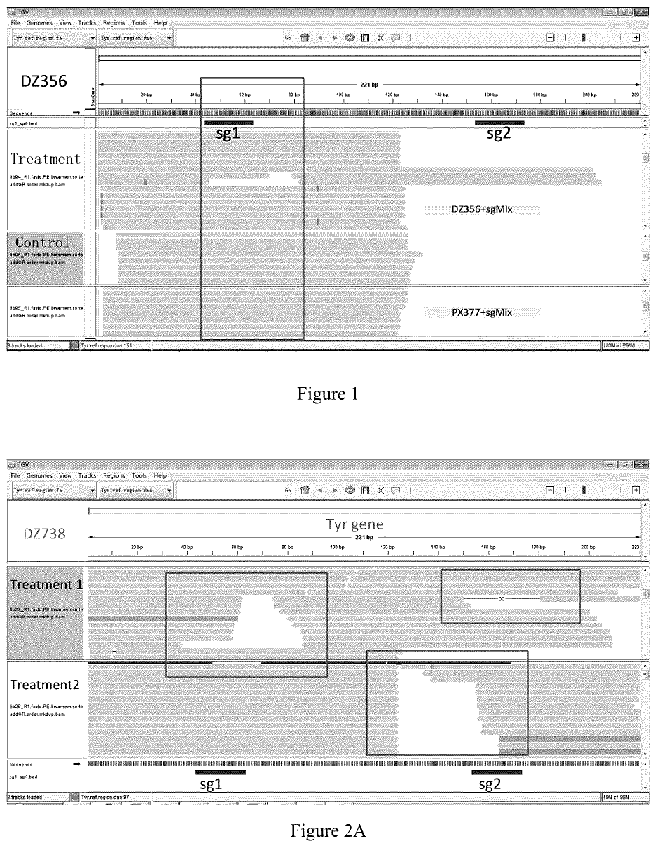

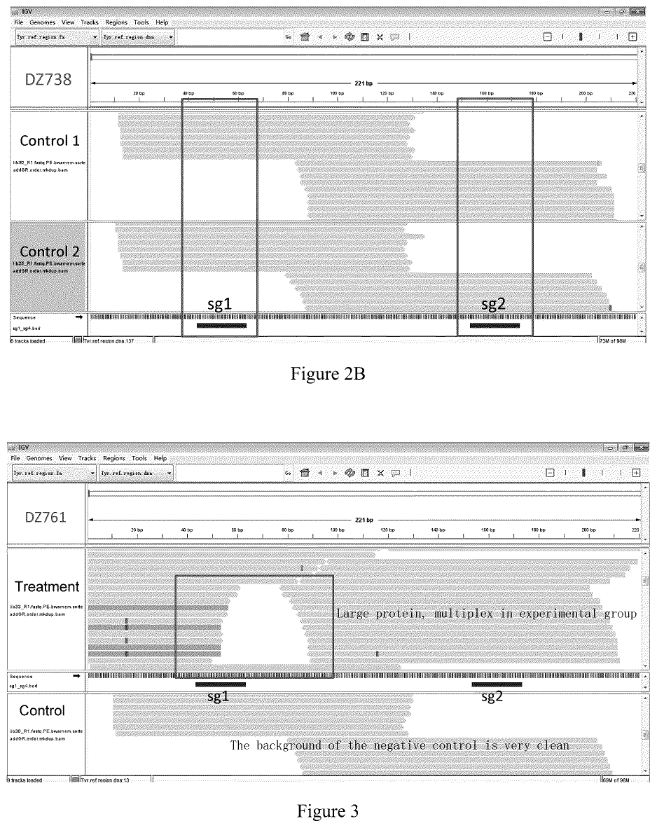

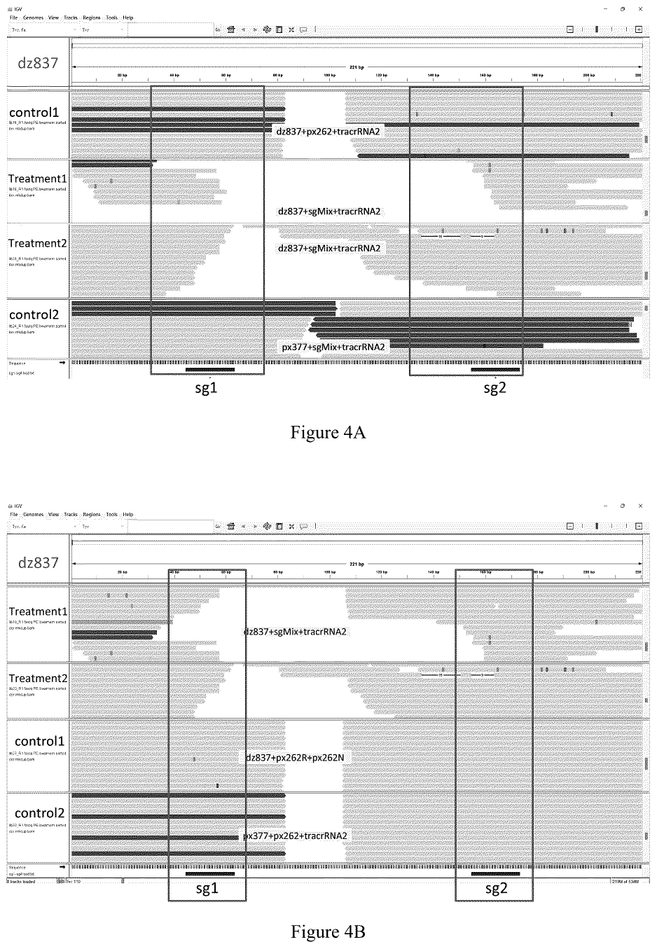

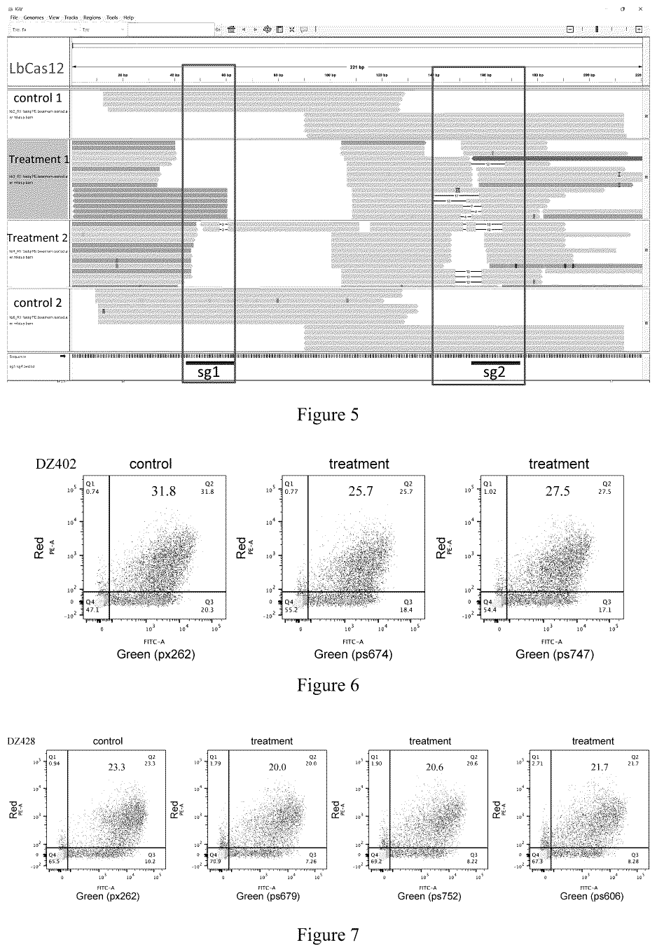

The sequencing results are aligned to TYR sequences near sg1 and sg2 that target the TYR gene. By removing redundancy and amplifying the sequences through PCR, bam file suitable for IGV visualization is finally obtained. FIG. 5 shows the result of the positive control LbCas12 cutting the endogenous gene TYR in 293T cell. It can be found that many cleavages and indel are observed near both sg1 and sg2 in treatment group, while the control groups are not observed. These indicate that the system we constructed for cleaving endogenous genes in eukaryotic cells is reliable. As further shown from FIGS. 1 to 4, near the sgRNA, candidate proteins DZ356, DZ738, DZ761, and DZ837 have a certain degree of cleavages and partial indels in treatment groups, while the background in control groups is very clean, and in the vicinity of sgRNA designed for the TYR gene, almost no cleavage occurs. These indicate that candidate proteins have the ability to cleave DNA.

### Example 3: Using Candidate Proteins to Knockdown mCherry Fluorescent Protein

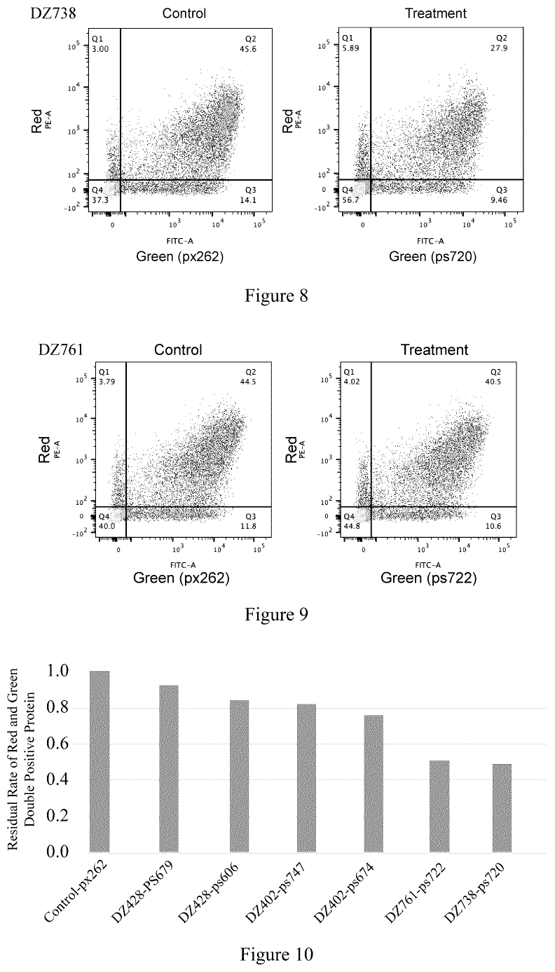

In order to test the effect of the candidate protein in cutting exogenously expressed genes, a plasmid expressing mCherry (emitting red light) was constructed, and all-in-one plasmid expressing the candidate protein and the corresponding targeting mCherry (the plasmid carries GFP fluorescent protein) were also constructed. The above all-in-one plasmid uses the CMV promoter to drive the candidate protein and simultaneously expresses GFP green light through T2A. At the same time, the u6 promoter is used to start the sgRNA targeting mCherry; and the two are constructed into one plasmid, which is all-in-one plasmid. The 293T cell line is transiently transfected, and flow cytometric analysis is performed after 72 hours. The results are shown from FIGS. 6 to 9. Compared with the control group, the treatment groups of DZ402, DZ428, DZ738, and DZ761 can affect the expression of mCherry to a certain extent. The remaining ratio of dual red and green fluorescence (in the Q2 quadrant of flow cytometry analysis) are further analysed after the candidate protein cleaves mCherry, the results are shown in FIG. 10.

### Example 4: Verification of Cleavage Activity of Candidate Proteins in Prokaryotes

Referring to Lee, J. K., Jeong, E., Lee, J. et al. Nat Commun 9, 3048 (2018), the method of forward screening of Cas9 protein mutants in Escherichia coli was used. Through introducing and inducibly expressing a lethal toxin protein gene named ccdb. (source from Bernard, P. and M. Couturier, Cell killing by the F plasmid CcdB protein involves poisoning of DNA-topoisomerase II complexes. J Mol Biol, 1992.226 (3): p.735-45), E. coli was caused to die. When the cleavage target of the CRISPR/Cas system is set on the ccdb lethal protein gene, once the cleavage occurs, E. coli will survive and grow monoclonal colonies.

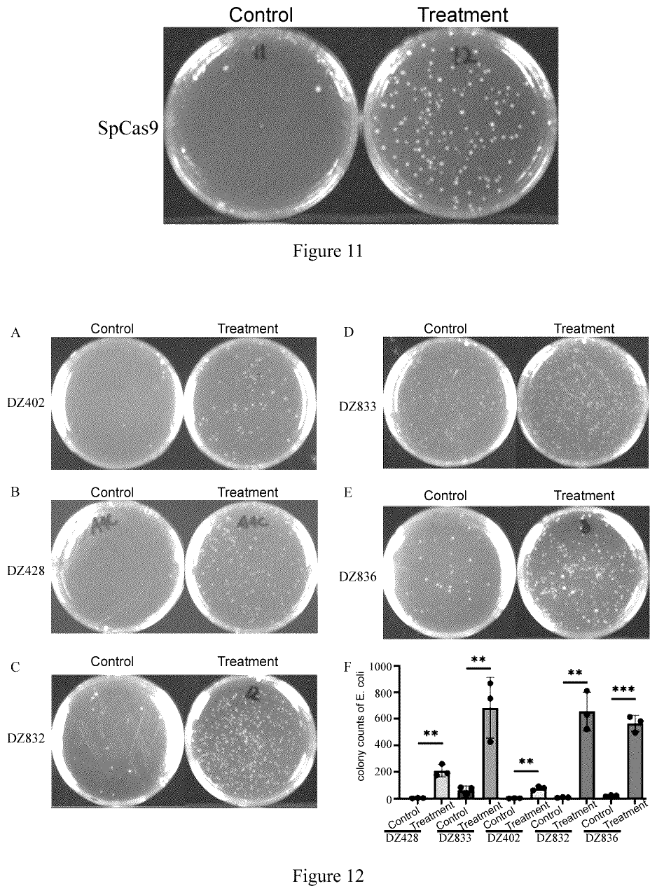

FIG. 11 shows the experimental results of using positive protein (SpCas9) to cleave toxic proteins. The treatment group shows many monoclonal colonies, while the control group shows almost no monoclonal colony, which indicate that the forward screening system we constructed is reliable.

FIG. 12 shows the experimental results of forward screening of DNA digestion by using the candidate proteins DZ402, DZ428, DZ832, DZ833 and DZ836. The culture media of DZ402, DZ428, DZ832, DZ833 and DZ836 all produced a large number of monoclonal colonies, which indicate that they can effectively cleave the toxin protein ccdb, while the negative control medium produced few or almost no colonies.

The base sequence used to express lethal toxin protein ccdb in this example is:

(SEQ ID NO: 277)
atgcagtttaaggtttacacctataaaagagagagccgttatcgt
ctgtttgtggatgtacagagtgatattattgacacgcccgggcga
cggatggtgatccccctggccagtgcacgtctgctgtcagataaa
gtctcccgtgaactttacccggtggtgcatatcggggatgaaagc
tggcgcatgatgaccaccgatatggccagtgtgccggtctccgtt
atcggggaagaagtggctgatctcagccaccgcgaaaatgacatc
aaaaacgccattaacctgatgttttggggaatataa

### Example 13: PAM Screening of Novel Candidate Proteins

For the functional proteins with cleavage activity obtained through the above screening, we further examined the PAM (Protospacer Adjacent Motif) of the candidate proteins through a negative selection method in Escherichia coli, with reference to Zetsche et al., 2015, Cell 163, 759-771. Due to E. coli has basically no double-strand break repair mechanism, if cleavage occurs on the plasmid, it will appear as plasmid deletion. Therefore, a PAM library consisting of 6 N can be designed with antibiotic resistance on the plasmid of this PAM library. The targeting sequence of the CRISPR/cas system is designed on the plasmid of the 6N PAM library. Cleavage by the Cas protein at specific PAMs will lead to plasmid loss, resulting in the corresponding loss of antibiotic resistance, then E. coli will not survive on the medium containing antibiotics. But the unmatched PAM will not be cleaved and the corresponding E. coli can survive. The final manifestation is that the number of E. coli monoclonal colonies in treatment group is less than that in the control group. By further performing amplicon sequencing on these bacterial colonies and then comparing the differential PAMs between the treatment and control groups, the sequence preference motif of the nucleic acid cleaved by the protein can be obtained.

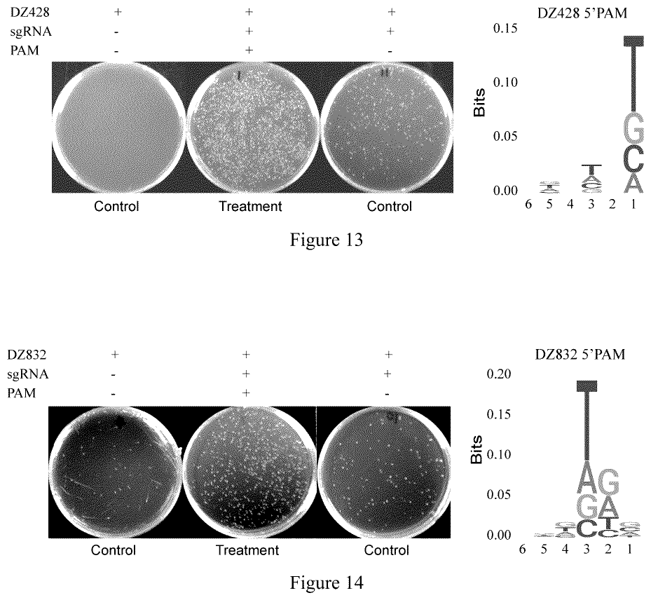

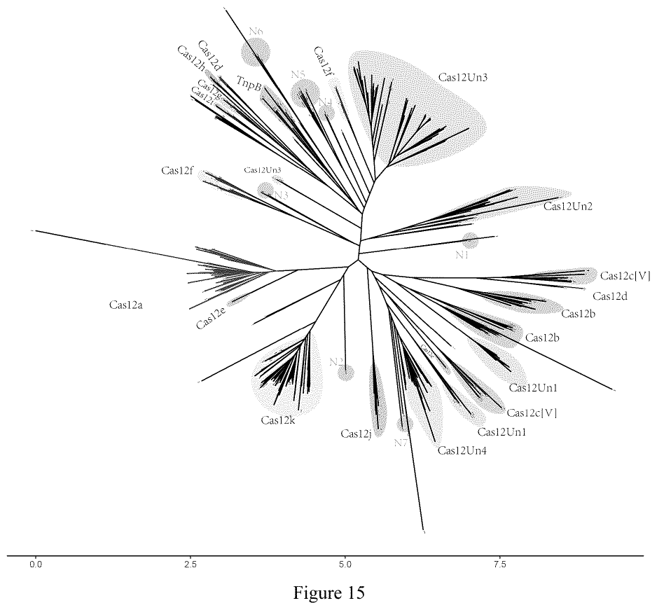

The experimental results show that DZ428 and DZ832 have PAM. FIG. 13 shows the PAM screening results of DZ428. It can be seen that there is a significant difference of E. coli clones in treatment group and control group. Through second-generation sequencing and analysis, it is found that the protein has a significant base sequence preference at the 5′ end, its potential motif is 5′-NNNNNT-Spacer-3′, wherein N represents any one of A, T, C or G. FIG. 14 shows the PAM screening results of DZ832. It can be seen that there is a significant difference of E. coli clones in treatment group and control group. Through second-generation sequencing and analysis, it is found that the protein has a significant base sequence preference at the 5′ end, and its potential motif is 5′-NNNTNN-Spacer-3′, wherein N represents any one of A, T, C or G.

### Example 6: DNA Nucleic Acid Detection Function of New Candidate Cas12 Protein

In view of the very strong non-specific bystander DNase activity of the candidate Cas 12 protein, it can potentially be used in the detection of DNA, such as DNA viruses and tumor signaling DNA molecules. In brief, a CRISPR-Cas system capable of cleaving target detection nucleic acids is constructed (for example, it can be in the form of a test strip, or coated with a delivery vector, etc.), including the candidate CRISPR-Cas12 protein, sgRNA (targeting the virus DNA for detection), and reporter detection molecules (such as DNA fluorescent reporter molecules). When the system binds to the target DNA, it plays a role of the bystander DNase activity of the candidate Cas12 protein and lead to cleave the reporter detection molecules, thereby causing the signal molecules to emit signals, such as fluorescence. These signals can be received by the detection instrument and converted into electrical signals for reading, thus achieving the purpose of detecting the target nucleic acid. If the machine learning algorithm model is further integrated, the target nucleic acid can be further quantified and predicted. Therefore, it can be widely used in virus detection, such as HPV virus detection; it can also be widely used in non-invasive diagnosis of diseases (such as tumors), such as liquid biopsy.

### Example 7: Validation of Base Editing Functions of Novel Compact Candidate Cas12 Proteins

Currently, there are two main systems used for single base editing, one is the ABE system and the other is the CBE system. In brief, by mutating the DNA cleavage domains (RuvC domain and/or HNH domain) of candidate Cas12 proteins, candidate dCas12 proteins with DNA binding ability but no cleavage activity is obtained. Subsequently, by fusing the adar enzyme sequence, a plasmid for the ABE (adenine base editor) single-base editing system is constructed. Then, specific sgRNA is designed for base directed mutations in a particular sequence, such as the TYR gene, and the corresponding plasmid vector is constructed. After that, the human 293T cell line was co-transfected, and flow cytometry was performed 48 hours later to obtain the co-transfected cell line. Primers are designed at upstream and downstream 50 bp region of the sgRNA, and the target DNA fragment is amplified. Following this, a deep-sequencing library is constructed and sequenced. After sequencing, bioinformatics methods are used to analyze the DNA mutations status near the sgRNA of the TYR gene, allowing for the assessment of the single-base editing efficiency of the corresponding ABE system. Thus, through continuous optimization of sgRNA, the optimal single base editing system for the target region can be constructed.

### Example 8: Homology Analysis of Candidate Cas12 Proteins and Known Cas12 Proteins

This is based on the principle that the higher the coverage and the greater similarity proportion between an unknown protein and a known protein, the closer homology between the unknown protein and the known protein. For the screened the candidate proteins, we first downloaded the relevant Cas12 protein sequences, such as LbCas12a, etc., from the NCBI database and patent. These sequences are then merged with our data to build a local blastp index file. Subsequently, the candidate protein sequences are aligned to this local blastp index database for protein sequence alignment analysis. For the part that the protein identity is less than 20% or cannot be compared to the local index library, we mark it as 20%; similarly, for the part that the coverage is less than 5% or cannot be compared to the local index library, we marked it as 1%. The homology level of the newly identified Cas12 proteins by the method of the present invention is very low compared to the known Cas12 proteins of various families. For example, the identity of DZ318, DZ319, DZ325, etc. is less than 65% compared with the known Cas12 categories. Additionally, some proteins also show very low identity to the DNA nuclease TnpB that relies on guide RNA guidance, such as, the identity of DZ380, DZ837, DZ845, etc. is less than 60% compared with currently known TnpB categories.

Through evolutionary lineage analysis (shown in FIG. 15), we also found that in addition to extending the members of cas12 protein family, there are seven new families that are evolutionarily distant from the currently known Cas12 family proteins, and the amino acid length of these family proteins is generally below 400 amino acids. These discoveries have broadened our understanding of the ultra-small Cas 12 protein family.

### Example 9: Preference Optimization

Based on past research, the Cas12 protein may have a strong preference (PAM) when targeting DNA sequences. Therefore, better cleavage activity results can be obtained by further optimizing the preference of the cas12 protein disclosed in the present application.

The DR sequence of the candidate Cas12 protein is shown in Table 1 below.

TABLE 1
DR sequences of candidate Cas12 proteins

| SEQ_ | | |
|---|---|---|
| ID_ | | |
| NO | DR-ID | DR-SEQ |
| 105 | dz117a | ATTTCAACTCACGCCTTCACGGAAGGCGAC |
| 106 | dz117b | GTCGCCTTCCGTGAAGGCGTGAGTTGAAAT |
| 107 | dz318a | GTCAGTAGGGCAGCAAGGATTAGCAGCCGT |
| | | TGACAC |
| 108 | dz318b | GTGTCAACGGCTGCTAATCCTTGCTGCCCT |
| | | ACTGAC |
| 109 | dz319a | GCTTCAGTCGGCGCGAATTCAGGAATCGTG |
| | | AGGCGG |
| 110 | dz319b | CCGCCTCACGATTCCTGAATTCGCGCCGAC |
| | | TGAAGC |
| 111 | dz325a | CCTGTATGTACATACAAAAATAGGTGCAGA |
| | | AACAGTT |
| 112 | dz325b | AACTGTTTCTGCACCTATTTTTGTATGTAC |
| | | ATACAGG |
| 113 | dz326a | ATCTACGAAAGTAGAAATTCTTAAAGAGCT |
| | | TTAGCC |
| 114 | dz326b | GGCTAAAGCTCTTTAAGAATTTCTACTTTC |
| | | GTAGAT |
| 115 | dz330a | GCTGTATGTTACCCACATT |
| 116 | dz330b | AATGTGGGTAACATACAGC |
| 117 | dz331a | AGTTGTATATACCTTTCATATTAAGGTGAA |
| | | TTACACC |
| 118 | dz331b | GGTGTAATTCACCTTAATATGAAAGGTATA |
| | | TACAACT |
| 119 | dz332a | GTTGTAATTACATTAAAAACCGAAGGTTGA |
| | | TACAAC |
| 120 | dz332b | GTTGTATCAACCTTCGGTTTTTAATGTAAT |
| | | TACAAC |
| 121 | dz333a | GTCTGCTGTATATACCCTTCATTCTTAGGT |
| | | GTGTAACACCCT |
| 122 | dz333b | AGGGTGTTACACACCTAAGAATGAAGGGTA |
| | | TATACAGCAGAC |
| 123 | dz337a | GTCGCGATCGCCGTTTCAGGGCAGGTCGAA |
| | | TTGAAAGT |
| 124 | dz337b | ACTTTCAATTCGACCTGCCCTGAAACGGCG |
| | | ATCGCGAC |
| 125 | dz339a | GTTTTGTTACCAGTACAAATAGAACAGTTC |
| | | CAAAAC |
| 126 | dz339b | GTTTTGGAACTGTTCTATTTGTACTGGTAA |
| | | CAAAAC |
| 127 | dz349a | CTTTCAGCCCACCCATTTCTGGGAAGGCGA |
| | | GGGCGGC |
| 128 | dz349b | GCCGCCCTCGCCTTCCCAGAAATGGGTGGG |
| | | CTGAAAG |
| 129 | dz356a | GCCGCAGTCGCCGCGATTTCAGGAAGCATG |
| | | AGGCGG |
| 130 | dz356b | CCGCCTCATGCTTCCTGAAATCGCGGCGAC |
| | | TGCGGC |
| 131 | dz362a | GTAGAAACCAACAGGGTTTCTGGAAAGGGA |
| | | TTGAAAG |
| 132 | dz362b | CTTTCAATCCCTTTCCAGAAACCCTGTTGG |
| | | TTTCTAC |
| 133 | dz377a | CTTCCAATCAGCCGATCGCTTAACCAGAAG |
| | | CTTCACC |
| 134 | dz377b | GGTGAAGCTTCTGGTTAAGCGATCGGCTGA |
| | | TTGGAAG |
| 135 | dz380a | CCACAACCCCACCAGGTTCC |
| 136 | dz380b | GGAACCTGGTGGGGTTGTGG |
| 137 | dz381a | GGCTGCTCCGGGTGCGCGTGGAGCGAGG |
| 138 | dz381b | CCTCGCTCCACGCGCACCCGGAGCAGCC |
| 139 | dz382a | CTTTCAATCTGCTCGTTGCTGAAAAGGCGA |
| | | TCGCGAC |
| 140 | dz382b | GTCGCGATCGCCTTTTCAGCAACGAGCAGA |
| | | TTGAAAG |
| 141 | dz383a | GTTGCATCCGCTTTCCAGCAACCAGGGCGG |
| | | GTGAAAG |
| 142 | dz383b | CTTTCACCCGCCCTGGTTGCTGGAAAGCGG |
| | | ATGCAAC |
| 143 | dz390a | GTTTCAACTTTCCTTCCAGCTAGAGGCGGG |
| | | TTGAAAG |
| 144 | dz390b | CTTTCAACCCGCCTCTAGCTGGAAGGAAAG |
| | | TTGAAAC |
| 145 | dz391a | GTTGCACTCTCTGAATTAACGACGTGAACG |
| | | ATGCAAC |
| 146 | dz391b | GTTGCATCGTTCACGTCGTTAATTCAGAGA |
| | | GTGCAAC |
| 147 | dz392a | CTCAAATACCAATAATAAATTTCTACTTTT |
| | | GTAGAT |
| 148 | dz392b | ATCTACAAAAGTAGAAATTTATTATTGGTA |
| | | TTTGAG |
| 149 | dz394a | GTCACAATCAGCTGTCCCCATGAGGGCAGG |
| | | TTGAAAG |
| 150 | dz394b | CTTTCAACCTGCCCTCATGGGGACAGCTGA |
| | | TTGTGAC |
| 151 | dz395a | GGTTCCTCCGCTCACGCGGAGATGCGCC |
| 152 | dz395b | GGCGCATCTCCGCGTGAGCGGAGGAACC |
| 153 | dz402a | CTTTCAACCCACCCTGTTGCGGGAGGGTTG |
| | | ATGAAAC |
| 154 | dz402b | GTTTCATCAACCCTCCCGCAACAGGGTGGG |
| | | TTGAAAG |
| 155 | dz403a | CTTTGGTTGCGATCGCCTTTTCAGCAGACG |
| | | GCGGATTGAAAGAG |
| 156 | dz403b | CTCTTTCAATCCGCCGTCTGCTGAAAAGGC |
| | | GATCGCAACCAAAG |
| 157 | dz404a | CTTTCCACTGACCAAATCCCCGCAAGGGGA |
| | | CGGAAAC |
| 158 | dz404b | GTTTCCGTCCCCTTGCGGGGATTTGGTCAG |
| | | TGGAAAG |
| 159 | dz413a | GGTCGCGATCGCCTTTTCAGCAACGAGCAG |
| | | ATTGAAAGC |
| 160 | dz413b | GCTTTCAATCTGCTCGTTGCTGAAAAGGCG |
| | | ATCGCGACC |
| 161 | dz418a | GTCTATAAAAGGTTTTAAATTTCTACTATT |
| | | GTAGAT |
| 162 | dz418b | ATCTACAATAGTAGAAATTTAAAACCTTTT |
| | | ATAGAC |
| 163 | dz425a | GTTGCAATCGCCTTCCCAGAGATGGGTGGG |
| | | CTGAAAG |
| 164 | dz425b | CTTTCAGCCCACCCATCTCTGGGAAGGCGA |
| | | TTGCAAC |
| 165 | dz428a | CTTTCAGCCCTCCCTATCACTAGGAGGGCG |
| | | ATTGCGAC |
| 166 | dz428b | GTCGCAATCGCCCTCCTAGTGATAGGGAGG |
| | | GCTGAAAG |
| 167 | dz433a | GTCGCAACTGCCCCTTCATTCGAAGGGAGG |
| | | TTGAAAG |
| 168 | dz433b | CTTTCAACCTCCCTTCGAATGAAGGGGCAG |
| | | TTGCGAC |
| 169 | dz446a | GGTTTGCTGACCTCGATTGTTGGAGGAGTG |
| | | CAAAAG |
| 170 | dz446b | CTTTTGCACTCCTCCAACAATCGAGGTCAG |
| | | CAAACC |
| 171 | dz447a | TACAATACCTATTAGGGATTGAAAG |
| 172 | dz447b | CTTTCAATCCCTAATAGGTATTGTA |
| 173 | dz449a | GTTTCAACACCCCTCCCAGCGAGAGGCGGG |
| | | TTGAAAG |
| 174 | dz449b | CTTTCAACCCGCCTCTCGCTGGGAGGGGTG |
| | | TTGAAAC |
| 175 | dz452a | CCGCCTCATGCCGACTGAAATCACGGGGAG |
| | | TGCGAC |
| 176 | dz452b | GTCGCACTCCCCGTGATTTCAGTCGGCATG |
| | | AGGCGG |
| 177 | dz454a | GTCTGAAGGCCTAAAGGAAGCTGAATCTGC |
| | | TGGCAC |
| 178 | dz454b | GTGCCAGCAGATTCAGCTTCCTTTAGGCCT |
| | | TCAGAC |
| 179 | dz472a | GTGTCAGTCGATCAGGAGCGAGCCGGCTCG |
| | | CTCCGC |
| 180 | dz472b | GCGGAGCGAGCCGGCTCGCTCCTGATCGAC |
| | | TGACAC |
| 181 | dz488a | GTGCCATTACCTTACGCTTCTGCAAAGCGT |
| | | TCATGC |
| 182 | dz488b | GCATGAACGCTTTGCAGAAGCGTAAGGTAA |
| | | TGGCAC |
| 183 | dz499a | GCCTGACCGCCCCGATCAATGGTGGATAGC |
| | | TGGCAC |
| 184 | dz499b | GTGCCAGCTATCCACCATTGATCGGGGCGG |
| | | TCAGGC |
| 185 | dz502a | GTCTTAGGGCATAAAGGAAATTGAATTTGC |
| | | TGGCAC |
| 186 | dz502b | GTGCCAGCAAATTCAATTTCCTTTATGCCC |
| | | TAAGAC |
| 187 | dz503a | GTGCCAGCTTTCATCCAATGGCCATGAAGG |
| | | AAGGAC |
| 188 | dz503b | GTCCTTCCTTCATGGCCATTGGATGAAAGC |
| | | TGGCAC |
| 189 | dz510a | GATCTTCCTAATCGATCAATTGTGGATAGC |
| | | TGGCAC |
| 190 | dz510b | GTGCCAGCTATCCACAATTGATCGATTAGG |
| | | AAGATC |
| 191 | dz511a | TGTACACATATATAATTTTTACTCTAACGT |
| | | AAAT |
| 192 | dz511b | ATTTACGTTAGAGTAAAAATTATATATGTG |
| | | TACA |
| 193 | dz574a | ATCTACAACAGTAGAAATTTGGTGGTACCT |
| | | TTAGAC |
| 194 | dz574b | GTCTAAAGGTACCACCAAATTTCTACTGTT |
| | | GTAGAT |
| 195 | dz599a | GTCTAATACCTATATAAAATTTCTACTTTT |
| | | GTAGAT |
| 196 | dz599b | ATCTACAAAAGTAGAAATTTTATATAGGTA |
| | | TTAGAC |
| 197 | dz649a | ACCGCAGGGCCTTTGGATTTGAAGAGCTTG |
| | | TGGCGG |
| 198 | dz649b | CCGCCACAAGCTCTTCAAATCCAAAGGCCC |
| | | TGCGGT |
| 199 | dz654a | GCCGCAGTCGCCGCGACTGCGGCAATCTTG |
| | | AGGCGG |
| 200 | dz654b | CCGCCTCAAGATTGCCGCAGTCGCGGCGAC |
| | | TGCGGC |
| 201 | dz655a | CCGCCACAAGCTCTTCAAAGTCTCAGGCCC |
| | | TGCGAC |
| 202 | dz655b | GTCGCAGGGCCTGAGACTTTGAAGAGCTTG |
| | | TGGCGG |
| 203 | dz680a | CCCGCGCTCGATGCGATGTCAGCGAGACTG |
| | | AGGCGG |
| 204 | dz680b | CCGCCTCAGTCTCGCTGACATCGCATCGAG |
| | | CGCGGG |
| 205 | dz697a | GCCGCAGTCGCCGCGAATCCGGGGGGCGTG |
| | | AGGCGG |
| 206 | dz697b | CCGCCTCACGCCCCCCGGATTCGCGGCGAC |
| | | TGCGGC |
| 207 | dz699a | CCGCCTGAAGCTCTTCGGAAACGCGAGGCG |
| | | TTCAAC |
| 208 | dz699b | GTTGAACGCCTCGCGTTTCCGAAGAGCTTC |
| | | AGGCGG |
| 209 | dz712a | CCGCCTCATGCTGTCTGAAATCGCGCCGTG |
| | | TGCGCC |
| 210 | dz712b | GGCGCACACGGCGCGATTTCAGACAGCATG |
| | | AGGCGG |
| 211 | dz715a | GTTCCTTCCGCAGTGATTTCTAAAAGCTTG |
| | | AGGCGG |
| 212 | dz715b | CCGCCTCAAGCTTTTAGAAATCACTGCGGA |
| | | AGGAAC |
| 213 | dz720a | GCCGCAGTCGCCGCGATTCCGGGGGAAGTG |
| | | AGGC |
| 214 | dz720b | GCCTCACTTCCCCCGGAATCGCGGCGACTG |
| | | CGGC |
| 215 | dz723a | CCGCCTCACGCTCTTCGAAACCGCGGGATG |
| | | TGCGAC |
| 216 | dz723b | GTCGCACATCCCGCGGTTTCGAAGAGCGTG |
| | | AGGCGG |
| 217 | dz729a | GTCCCCCCCGTAGCGATCTCAGGAAACGTG |
| | | AGGCGG |
| 218 | dz729b | CCGCCTCACGTTTCCTGAGATCGCTACGGG |
| | | GGGGAC |
| 219 | dz736a | GGTGCCGACGTCGGCACCAACCTGATCGAC |
| | | GGACAC |
| 220 | dz736b | GTGTCCGTCGATCAGGTTGGTGCCGACGTC |
| | | GGCACC |
| 221 | dz738a | GGTGCCGACGTCGGCACCAACCTGATCGAC |
| | | GGACAC |
| 222 | dz738b | GTGTCCGTCGATCAGGTTGGTGCCGACGTC |
| | | GGCACC |
| 223 | dz757a | GTGGCAGAAGTCGGAACCGATCTGATCAAC |
| | | TGACAC |
| 224 | dz757b | GTGTCAGTTGATCAGATCGGTTCCGACTTC |
| | | TGCCAC |
| 225 | dz761a | GGCTCCTACGTTGAAACTAGCCTGATCGGC |
| | | TGACAC |
| 226 | dz761b | GTGTCAGCCGATCAGGCTAGTTTCAACGTA |
| | | GGAGCC |
| 227 | dz875a | CCCGTTGCAATTCATGGGGAGTGGAGTGGT |
| | | ATCATGAAAG |
| 228 | dz875b | CTTTCATGATACCACTCCACTCCCCATGAA |
| | | TTGCAACGGG |
| 229 | dz876a | TCAATTCTATAGTAGATCATC |
| 230 | dz876b | GATGATCTACTATAGAATTGA |
| 231 | dz877a | GATGATCTACTATAGAATTGA |
| 232 | dz877b | TCAATTCTATAGTAGATCATC |
| 233 | dz878a | GTCCCCACTCGCTGGGGACATTAATTGAAT |
| | | GGAAAC |
| 234 | dz878b | GTTTCCATTCAATTAATGTCCCCAGCGAGT |
| | | GGGGAC |
| 235 | dz879a | GCCTAAGAGCATAATGAAATTTCTACTGTT |
| | | GTAGAT |
| 236 | dz879b | ATCTACAACAGTAGAAATTTCATTATGCTC |
| | | TTAGGC |
| 237 | dz880a | GCTTCAACCCCTCAAGGGTGCATCTGAAAC |
| 238 | dz880b | GTTTCAGATGCACCCTTGAGGGGTTGAAGC |
| 239 | dz881a | GCTTCAAACCCACAAGGGTTCATCTGAAAC |
| 240 | dz881b | GTTTCAGATGAACCCTTGTGGGTTTGAAGC |
| 241 | dz882a | GCTTCAACCCCTCAAGGGTTCATCTGGAAC |
| 242 | dz882b | GTTCCAGATGAACCCTTGAGGGGTTGAAGC |
| 243 | dz883a | GTTTCAGCCGAACCCTTGAGGGGTTGCAGT |
| 244 | dz883b | ACTGCAACCCCTCAAGGGTTCGGCTGAAAC |
| 245 | dz884a | ATCTACGAGAGTAGAAATTAACATATACTG |
| | | TCAGA |
| 246 | dz884b | TCTGACAGTATATGTTAATTTCTACTCTCG |
| | | TAGAT |
| 247 | dz885a | GTCGCTCCCCGCTCGGGGAGCGTGAGTTGA |
| | | AAC |
| 248 | dz885b | GTTTCAACTCACGCTCCCCGAGCGGGGAGC |
| | | GAC |
| 249 | dz886a | GTCACAGCTACCTTCCAAATAGGCACCTGC |
| | | TTTAGC |
| 250 | dz886b | GCTAAAGCAGGTGCCTATTTGGAAGGTAGC |
| | | TGTGAC |
| 251 | dz887a | ATCTACGAAAGTAGAAATTCTTAAAGAGCT |
| | | TTAGCC |
| 252 | dz887b | GGCTAAAGCTCTTTAAGAATTTCTACTTTC |
| | | GTAGAT |
| 253 | dz888a | GTTTTGAAGTGTTAAATAATATCTACTATT |
| | | GTAGAT |
| 254 | dz888b | ATCTACAATAGTAGATATTATTTAACACTT |
| | | CAAAAC |
| 255 | dz889a | CTTTCAATCCCTTATAGGTAGGCTAAAAAC |
| 256 | dz889b | GTTTTTAGCCTACCTATAAGGGATTGAAAG |
| 257 | dz890a | TCCGTTTCAAACGAACCCATGAGGGGTTGA |
| | | AGC |
| 258 | dz890b | GCTTCAACCCCTCATGGGTTCGTTTGAAAC |
| | | GGA |
| 259 | dz891a | GGCTATAAAGCCATTTTAATTTCTACTATT |
| | | GTAGAT |
| 260 | dz891b | ATCTACAATAGTAGAAATTAAAATGGCTTT |
| | | ATAGCC |
| 261 | dz892a | GGTGATATAGTAACTGGTCTGTTCCAGCAC |
| | | TTCACC |
| 262 | dz892b | GGTGAAGTGCTGGAACAGACCAGTTACTAT |
| | | ATCACC |
| 269 | dz832a | ATCTACAACAGTAGAAATTAAAACGGGTAT |
| | | TAAAAC |
| 270 | dz832b | GTTTTAATACCCGTTTTAATTTCTACTGTT |
| | | GTAGAT |
| 271 | dz833a | GAATCCATACAATGGAATTGAAAG |
| 272 | dz833b | CTTTCAATTCCATTGTATGGATTC |
| 273 | dz836a | ACTTCACCCTGCACTCCGTTCGTCACCTGC |
| | | TCACGC |
| 274 | dz836b | GCGTGAGCAGGTGACGAACGGAGTGCAGGG |
| | | TGAAGT |
| 275 | dz837a | CTTTCAATTCTATAGTAGATCATCT |
| 276 | dz837b | AGATGATCTACTATAGAATTGAAAG |

For a summary table of tracrRNA sequence information of candidate proteins, see Table 2.

TABLE 2
tracrRNA coding sequence of
candidate Cas12 proteins

| SEQ_ | | |
|---|---|---|
| ID_ | tracrRNA_ | |
| NO | ID | TracrRNA seq (5->3) |
| 263 | dz349 | TTTCAGCAGCACAAGGTCGGTGACATCTTC |
| | | ATCACGAGCTACGGCAGCCTGGATCGCCTG |
| | | AAAGTTTTGCTGACCTGTTTGGCGATTTTT |
| | | GTCGTAC |
| 264 | dz356 | TTCCTGAAAAATATTCTCGACCCATTGATA |
| | | GTAGAACAGGCGCGGACAATAGATGAAGTT |
| | | GTGCAAGCGCCGCACTGGCAGAGGCGGCTG |
| | | GGCCTCGAGTGCGGTTTGGGTTTGGACCGG |
| | | CGCGTCGCTCATTACGCCTACATTGGGACA |
| | | TCGTCCTTCACTTGCTCATGATTCAGCCAC |
| | | CGCCGAAGCCGTTCGTTGTTGATGGCCTCG |
| | | CACCGCCTCCACTGGTATTCCCGATCATTC |
| | | ACGGTCTTCCAAACACCGGGACCGGAGGCG |
| | | TAGTCGAATCGCCCAGCCGAATCGGTTTCC |
| | | AGTCGCGCTCGCCCGAAAGGGGCGATTTCA |
| | | TGTCGGT |
| 265 | dz834 | TTTCAATTGTATACTGTAAGAACACTTTTT |
| | | GATGGATAGGGTGTCCATGGTAACGTATTT |
| | | AAGCTTTGGTTCATGTGGTGTTTTTAAGCT |
| | | TCCCATGTTCTGTGTGGGATGTTTTTGGTG |
| | | T |
| 266 | dz837a | TATAGAAACATTCCTACCCTAGAAGACGAG |
| | | AATTTAAGTTTAATAAAAAGCTAGCTCTTC |
| | | AAATCTCAGACTATAAGCTAGCTCTTCTTG |
| | | CTCTCTT |
| 267 | dz837b | CTACTATGTGCCCGCATGCTCTTATGTATC |
| | | GATCCAAGGGGATCCTTATGAGCTGAGGAT |
| | | CAATGTTGTTAATATTATAATGGCCATAAT |
| | | ACTTAGTAAGTTAAACATACCCTGAGGTTC |
| | | GGGTTTCATTGGGCTTGGCTTTGGCCAAGC |
| | | TCTATTCCCTTGGC |
| 268 | dz842 | CACATGCGGGGCAGACGTAAACGCGGTCTT |
| | | TGAGTTTCAGGCCGCTATGCACATGTCCGC |
| | | AGGCGTGGCAGGTCTTGGAACTCGGGTAGA |
| | | ATCGATCCGCGATGCGGAATTCAATCCCGA |
| | | TGACCCCTGCCTTGCGCCGTAGTTTCTCAC |
| | | GGAGGGAATAAAAGCGCTGCTGCGCCACGA |
| | | CCTTCG |

Please see Table 3 for the amino acid sequence number, length, domain superfamily type and other information of the final candidate Cas12 protein.

TABLE 3
Summary table of candidate Cas12 proteins

| | | Length of | Structural superfamily | Coverage | Cas12 |
|---|---|---|---|---|---|
| SEQ_ID_NO | ID | protein | types | of Cas12 | similarity |
| 1 | dz117 | 607 | cas12superfamily | cas12 1% | cas12 20% |
| 2 | dz318 | 1364 | cas12superfamily | cas12 93% | cas12 36.38% |
| 3 | dz319 | 1344 | cas12superfamily | cas12 99% | cas12 47.20% |
| 4 | dz325 | 315 | HNH_5superfamily | cas12 1% | cas12 20% |
| 5 | dz326 | 317 | RuvC_1superfamily | cas12 98% | cas12 48.60% |
| 6 | dz330 | 374 | cas12superfamily | cas12 1% | cas12 20% |
| 7 | dz331 | 378 | cas12superfamily | cas12 1% | cas12 20% |
| 8 | dz332 | 378 | cas12superfamily | cas12 1% | cas12 20% |
| 9 | dz333 | 384 | cas12superfamily | cas12 1% | cas12 20% |
| 10 | dz337 | 409 | cas12superfamily | cas12 99% | cas12 68.38% |
| 11 | dz339 | 430 | cas12superfamily | cas12 1% | cas12 20% |
| 12 | dz349 | 472 | cas12superfamily | cas12 97% | cas12 76.25% |
| 13 | dz356 | 493 | cas12superfamily | cas12 47% | cas12 71.98% |
| 14 | dz362 | 516 | cas12superfamily | C2c8 67% | C2c8 36.96% |
| 15 | dz377 | 559 | cas12superfamily | C2c8 97% | C2c8 34.8% |
| 16 | dz380 | 572 | cas12superfamily | TnpB 78% | TnpB 23.58% |
| 17 | dz381 | 575 | cas12superfamily | cas14 88% | cas14 47.84% |
| 18 | dz382 | 588 | cas12superfamily | cas12 99% | cas12 76.28% |
| 19 | dz383 | 602 | cas12superfamily | cas12 99% | cas12 39.94% |
| 20 | dz390 | 615 | cas12superfamily | cas12 99% | cas12 74.51% |
| 21 | dz391 | 616 | cas12superfamily | cas14 98% | cas14 40.07% |
| 22 | dz392 | 617 | cas12superfamily | cas12 99% | cas12 47.35% |
| 23 | dz394 | 627 | cas12superfamily | cas12 99% | cas12 68% |
| 24 | dz395 | 628 | cas12superfamily | cas12 1% | cas12 20% |
| 25 | dz402 | 635 | cas12superfamily | cas12 99% | cas12 68.50% |
| 26 | dz403 | 635 | cas12superfamily | cas12 96% | cas12 75.41% |
| 27 | dz404 | 635 | cas12superfamily | cas12 99% | cas12 68.50% |
| 28 | dz413 | 638 | cas12superfamily | cas12 99% | cas12 74.41% |
| 29 | dz418 | 639 | RuvC_1superfamily | cas12 99% | cas12 68.29% |
| 30 | dz425 | 643 | cas12superfamily | cas12 97% | cas12 75.99% |
| 31 | dz428 | 645 | cas12superfamily | cas12 99% | cas12 54.75% |
| 32 | dz433 | 659 | cas12superfamily | cas12 99% | cas12 55.11% |
| 33 | dz446 | 709 | cas12superfamily | cas12 19% | cas12 28.28% |
| 34 | dz447 | 710 | cas12superfamily | C2c8 61% | C2c8 32.35% |
| 35 | dz449 | 723 | cas12superfamily | cas12 99% | cas12 74.03% |
| 36 | dz452 | 754 | cas12superfamily | cas12 97% | cas12 40.81% |
| 37 | dz454 | 788 | cas12superfamily | cas12 97% | cas12 42.17% |
| 38 | dz472 | 1471 | cas12superfamily | cas12 98% | cas12 62.05% |
| 39 | dz488 | 1128 | cas12superfamily | cas12 98% | cas12 45.41% |
| 40 | dz499 | 1150 | cas12superfamily | cas12 99% | cas12 52.69% |
| 41 | dz502 | 1164 | cas12superfamily | cas12 98% | cas12 39.16% |
| 42 | dz503 | 1165 | cas12superfamily | cas12 99% | cas12 54.57% |
| 43 | dz510 | 1194 | cas12superfamily | cas12 98% | cas12 54.46% |
| 44 | dz511 | 1198 | cas12superfamily | cas12 98% | cas12 30.58% |
| 45 | dz574 | 1257 | cas12superfamily | cas12 99% | cas12 71.89% |
| 46 | dz599 | 1277 | cas12superfamily | cas12 80% | cas12 46.16% |
| 47 | dz649 | 1323 | cas12superfamily | cas12 99% | cas12 37.02% |
| 48 | dz654 | 1332 | cas12superfamily | cas12 97% | cas12 47.41% |
| 49 | dz655 | 1332 | cas12superfamily | cas12 99% | cas12 36.44% |
| 50 | dz680 | 1366 | cas12superfamily | cas12 99% | cas12 41.48% |
| 51 | dz697 | 1649 | cas12superfamily | cas12 72% | cas12 34.69% |
| 52 | dz699 | 1390 | cas12superfamily | cas12 96% | cas12 42.19% |
| 53 | dz712 | 1818 | cas12superfamily | cas12 78% | cas12 38.24% |
| 54 | dz715 | 1423 | cas12superfamily | cas12 99% | cas12 56.41% |
| 55 | dz720 | 1429 | cas12superfamily | cas12 99% | cas12 66.47% |
| 56 | dz723 | 1435 | cas12superfamily | cas12 90% | cas12 34.72% |
| 57 | dz729 | 1453 | cas12superfamily | cas12 99% | cas12 36.02% |
| 58 | dz736 | 1470 | cas12superfamily | cas12 99% | cas12 74.58% |
| 59 | dz738 | 1474 | cas12superfamily | cas12 99% | cas12 76.42% |
| 60 | dz757 | 1519 | cas12superfamily | cas12 99% | cas12 58.05% |
| 61 | dz761 | 1537 | cas12superfamily | cas12 98% | cas12 57.54% |
| 62 | dz832 | 233 | RuvC_1superfamily | cas12 98% | cas12 52.74% |
| 63 | dz833 | 174 | OrfB/InsQ_superfamily | cas12 1% | cas12 20% |
| 64 | dz834 | 121 | OrfB/InsQ_superfamily | TnpB 95% | TnpB 63.48% |
| 65 | dz835 | 118 | OrfB/InsQ_superfamily | TnpB 71% | TnpB 61.9% |
| 66 | dz836 | 132 | OrfB/InsQ_superfamily | TnpB 68% | TnpB 31.52% |
| 67 | dz837 | 166 | OrfB/InsQ_superfamily | TnpB 98% | TnpB 49.7% |
| 68 | dz839 | 124 | OrfB/InsQ_superfamily | TnpB 95% | TnpB 76.27% |
| 69 | dz840 | 174 | OrfB/InsQ_superfamily | TnpB 45% | TnpB 77.22% |
| 70 | dz842 | 170 | OrfB/InsQ_superfamily | TnpB 98% | TnpB 70.24% |
| 71 | dz845 | 392 | cas12superfamily | TnpB 99% | TnpB 35.81% |
| 72 | dz846 | 390 | cas12superfamily | TnpB 97% | TnpB 55.53% |
| 73 | dz847 | 319 | cas12superfamily | cas12 98% | cas12 44.1% |
| 74 | dz848 | 382 | cas12superfamily | TnpB 81% | TnpB 48.3% |
| 75 | dz849 | 388 | cas12superfamily | TnpB 92% | TnpB 61.94% |
| 76 | dz850 | 373 | cas12superfamily | TnpB 40% | TnpB 30.23% |
| 77 | dz851 | 361 | cas12superfamily | TnpB 99% | TnpB 43.18% |
| 78 | dz852 | 389 | cas12superfamily | TnpB 93% | TnpB 44.38% |
| 79 | dz853 | 399 | cas12superfamily | TnpB 98% | TnpB 68.37% |
| 80 | dz854 | 398 | cas12superfamily | TnpB 88% | TnpB 47.6% |
| 81 | dz855 | 381 | cas12superfamily | TnpB 35% | TnpB 33.09% |
| 82 | dz856 | 365 | cas12superfamily | TnpB 99% | TnpB 35.81% |
| 83 | dz857 | 354 | cas12superfamily | TnpB 99% | TnpB 59.09% |
| 84 | dz859 | 392 | cas12superfamily | TnpB 98% | TnpB 65.98% |
| 85 | dz860 | 376 | cas12superfamily | TnpB 93% | TnpB 70.74% |
| 86 | dz861 | 387 | cas12superfamily | TnpB 93% | TnpB 44.38% |
| 87 | dz875 | 188 | cas12superfamily | TnpB 98% | TnpB 51.85% |
| 88 | dz876 | 105 | OrfB/InsQ_superfamily | TnpB 80% | TnpB 63.53% |
| 89 | dz877 | 105 | OrfB/InsQ_superfamily | TnpB 80% | TnpB 63.53% |
| 90 | dz878 | 133 | OrfB/InsQ_superfamily | cas12 1% | cas12 20% |
| 91 | dz879 | 193 | cas12superfamily | cas12 97% | cas12 46.07% |
| 92 | dz880 | 393 | cas12superfamily | TnpB 98% | TnpB 56.85% |
| 93 | dz881 | 390 | cas12superfamily | TnpB 97% | TnpB 55.26% |
| 94 | dz882 | 392 | cas12superfamily | TnpB 98% | TnpB 58.14% |
| 95 | dz883 | 347 | cas12superfamily | TnpB 90% | TnpB 60.31% |
| 96 | dz884 | 324 | cas12superfamily | cas12 66% | cas12 98.13% |
| 97 | dz885 | 347 | cas12superfamily | cas12 97% | cas12 46.78% |
| 98 | dz886 | 324 | cas12superfamily | TnpB 98% | TnpB 42.07% |
| 99 | dz887 | 317 | cas12superfamily | cas12 98% | cas12 48.60% |
| 100 | dz888 | 323 | cas12superfamily | cas12 87% | cas12 31.05% |
| 101 | dz889 | 387 | cas12superfamily | cas12 97% | cas12 57.22% |
| 102 | dz890 | 398 | cas12superfamily | TnpB 89% | TnpB 49.58% |
| 103 | dz891 | 370 | cas12superfamily | TnpB 99% | TnpB 76.96% |
| 104 | dz892 | 392 | cas12superfamily | TnpB 83% | TnpB 31.34% |

1-32. (canceled)

33. Cas12 protein, which comprises the amino acid sequence shown in any one from SEQ ID NO: 1 to 104, or a functional fragment thereof, or comprises the amino acid sequence of any one from SEQ ID NO: 1 to 104 with one or more amino acid substitutions, insertions, and/or deletions;
preferably, the protein has the activity for gene knock-in, gene knock-out, or gene modification of on DNA.

34. The Cas12 protein according to claim 33, the Cas12 protein has RuvC domain, Cas12 superfamily domain, and/or InsQ superfamily domain, and at least one amino acid of RuvC domain, Cas12 superfamily domain and/or InsQ superfamily domain has been further modified or engineered to reduce or eliminate its DNA cleavage activity, which results in dCas12 with reduced or abolished DNA cleavage activity;
preferably, the Cas12 protein is fused with one or more heterologous functional domains, wherein the fusion occurs at the N-terminal, C-terminal or internal part of the Cas12 protein;
more preferably, the heterologous functional domain is capable of cleaving one or more target sequences, or modifying the transcription or translation of the target sequence.

35. The Cas12 protein according to claim 34, wherein the one or more heterologous functional domains have the following activities: deaminase such as cytidine deaminase and deoxyadenosine deaminase, methylase, demethylase, transcriptional activation, transcriptional repression, nuclease, single-stranded DNA cleavage, double-stranded DNA cleavage, DNA or RNA ligase, reporter protein, detection protein, localization signal, or any combination thereof.

36. The Cas12 protein according claim 33, the Cas12 protein comprises RuvC domain, Cas12 superfamily domain, and/or InsQ superfamily domain;
preferably, the Cas12 protein comprises cas12k domain, cas12b domain, RuvC_1 domain, and/or OrfB/InsQ domain.

37. The Cas12 protein according to claim 36, wherein the substitution, insertion, and/or deletion of amino acids comprises performing substitution, insertion, and/or deletion in the RuvC domain, the Cas12 superfamily domain, and/or the InsQ superfamily domain;
preferably, after substitution, insertion, and/or deletion of one or more amino acids, the Cas12 protein exhibits reduced or eliminated activity for gene knock-in, gene knock-out, or modification on DNA.

38. A nucleic acid molecule, comprises a nucleotide sequence encoding the Cas12 protein according to claim 33;
preferably, the nucleic acid molecule is a nucleic acid molecule that has been codon-optimized for expression in host cell,
more preferably, wherein the host cell is prokaryotic cell or eukaryotic cell, even more preferably is animal cell, plant cell, or microbial cell.

39. The nucleic acid molecule according to claim 38, the nucleic acid molecule comprises a promoter operably linked to the nucleotide sequence encoding the Cas 12 protein, preferably, the promoter is a constitutive promoter, an inducible promoter, a tissue-specific promoter, a synthetic promoter, a chimeric promoter or development-specific promoter.

40. An expression vector, comprises the nucleic acid molecule according to claim 38;
preferably, further comprises crRNA sequence and/or tracr RNA sequence;
more preferably, further comprises a regulatory element that regulates the nucleic acid molecule, a regulatory element that regulates the crRNA sequence, and/or a regulatory element that regulates the tracr RNA sequence.

41. The expression vector according to claim 40, which is viral vector, nanoparticle, liposome nanoparticle (LNP), cationic polymer (e.g., PEI), liposome, exosome, virus-like particle (VLP), microvesicle or gene gun;
preferably, the viral vector includes: adeno-associated virus (AAV), recombinant adeno-associated virus (rAAV), adenovirus, lentivirus, retrovirus, herpes simplex virus or oncolytic virus.

42. CRISPR-Cas system, comprises: (1) the Cas12 protein or its derivative or its functional fragment according to claim 33, or a nucleic acid molecule, comprises a nucleotide sequence encoding the Cas 12 protein according to claim 33; (2) gRNA sequence for targeting target sequence.

43. The CRISPR-Cas system according to claim 42, the functional fragment of the Cas12 protein is a fragment of the amino acid shown in any one from SEQ ID NO: 1 to 104 with at least one amino acid deletion and retains the function of Cas12 protein; or
the derivative of the Cas12 protein is a protein that has more than 70%, 80%, 85%, 90%, 91%, 92%, 93%, 94%, 95%, 96%, 97%, 98%, or 99% identity to any one of the proteins or their functional fragments shown from SEQ ID NO: 1 to 104;
preferably, the derivative of the Cas 12 protein is a protein that has at least 1, 2, 3, 4, 5, 6, 7, 8, 9, or 10 amino acids insertion, deletion, and/or substitution based on the amino acid sequence shown in any one from SEQ ID NOs: 1 to 104.

44. The CRISPR-Cas system according to claim 43, wherein the gRNA sequence comprises a direct repeat (DR) sequence, a trans-acting CRISPR RNA (tracrRNA) and a spacer sequence targeting a target RNA segment.

45. The CRISPR-Cas system according to claim 44, wherein the DR sequence is the sequence shown in any one from SEQ ID NO: 105 to 262 or the sequence shown in any one from SEQ ID NO: 269 to 276.

46. The CRISPR-Cas system according to claim 44, wherein the tracrRNA sequence is the sequence shown in any one from SEQ ID NO: 263 to 268.

47. The CRISPR-Cas system according to claim 44, wherein the spacer sequence has 10-50 nucleotides, preferably has 15-25 nucleotides, more preferably has 20 nucleotides.

48. The CRISPR-Cas system according to claim 44, wherein the DR sequence is any one of the following derivatives (i)˜(iv), wherein,
the derivative (i) has one or more nucleotide additions, deletions, or substitutions compared to any of the sequences shown from SEQ ID NO: 105 to 262 or from SEQ ID NO: 269 to 276;
the derivative (ii) has at least 20%, 30%, 40%, 50%, 60%, 70%, 80%, 90%, 95% or 97% sequence identity to any of the sequences from SEQ ID NO: 105 to 262 or from SEQ ID NO: 269 to 276;
the derivative (iii) hybridizes to any of the sequences shown from SEQ ID NO: 105 to 262 or from SEQ ID NO: 269 to 276 or hybridizes to any of the sequences described in (i) and (ii) under stringent conditions; or
the derivative (iv) is a complement of any one of the derivatives (i)-(iii), provided that the derivative is not any of the sequences shown from SEQ ID NO: 105 to 262 or from SEQ ID NO: 269 to 276, and the derivative encodes RNA or itself is RNA, which maintains essentially the same secondary structure as any RNA encoded by SEQ ID NO: 105-262 or SEQ ID NO: 269-276.

49. The CRISPR-Cas system according to claim 48, wherein the tracrRNA sequence said includes a segment of pairing bases that are complementary to the described DR sequence in reverse, preferably the tracrRNA sequence and the DR sequence form at least 6, 8, 10 or 12 pairing bases, the said pairing bases is either continuous or spaced, preferably the tracrRNA sequence is the sequence shown in any one from SEQ ID NO: 263 to 268,
preferably,
wherein the tracrRNA is any one of the following derivatives (i) to (iv), wherein,
the derivative (i) has one or more (for example, 1, 2, 3, 4, 5, 6, 7, 8, 9, 10, 11, 12, 13, 14, 15, 16, 17, 18, 19 or 20) nucleotide additions, deletions, or substitution compared to any one of the sequences shown from SEQ ID NO: 263 to 268;
the derivative (ii) has at least 20%, 30%, 40%, 50%, 60%, 70%, 80%, 90%, 95% or 97% sequence identity to any one of the sequences shown from SEQ ID NO: 263 to 268;
the derivative (iii) hybridizes to any one of the sequences shown from SEQ ID NO: 263 to 268 or hybridizes to any of the sequences described in (i) and (ii) under stringent conditions; or
the derivative (iv) is the complement of any one of the derivatives (i)-(iii), provided that the derivative is not any of the sequences shown from SEQ ID NO: 263 to 268, and the derivative encodes RNA or itself is RNA, which maintains essentially the same secondary structure as any RNA encoded by SEQ ID NO: 263-268.

50. The CRISPR-Cas system according to claim 42, further comprises: (3) target DNA,
preferably, the CRISPR-Cas system can trigger the cleavage, sequence modification, single base editing, sequence insertion or deletion, sequence change or degradation, or the delivery of epigenetic modifiers or signals for transcriptional or translational activation or repression at or near the target DNA,
more preferably,
the target DNA is double-stranded DNA, single-stranded DNA, double-stranded circular DNA, or single-stranded circular DNA.

51. A method for degrading, cutting, or modifying a target DNA in a target cell, or delivering exogenous nucleic acids into or near the cells containing the target sequence, wherein the method comprises using the Cas 12 protein according to claim 33, or a nucleic acid molecule, comprises a nucleotide sequence encoding the Cas12 protein according to claim 33,
preferably, the target cell is prokaryotic cell or eukaryotic cell, preferably is animal cell, plant cell, or microbial cell,
more preferably, the target cell is in vitro cell or in vivo cell.

52. A method for detecting the target DNA, the target DNA is detected by the use of the Cas12 protein or its derivative or its functional fragment according to claim 33, the Cas12 protein or its derivative or its functional fragment expressed by a nucleic acid molecule, comprises a nucleotide sequence encoding the Cas12 protein according to claim 33,
preferably, the method further uses sgRNA targeting the target DNA and a reporter detection molecule, the Cas12 protein or its derivatives or its functional fragments is used to bind to the target DNA and then activate collateral DNA cleavage activity of the Cas12 protein, the activation results in the cleavage of the reporter molecule and allows for the detection of the signal emitted by the reporter molecule.
